SEQUENCE LISTING (358 sequences)

SEQ ID NO: 1 - protein, 18 aa, Artificial Sequence
  Description of Artificial Sequence Synthetic peptide
IRGWIFGTTLDSKTQSLL

SEQ ID NO: 2 - protein, 13 aa, Artificial Sequence
  Description of Artificial Sequence Synthetic peptide
CTFEYVSQPFLMD

SEQ ID NO: 3 - protein, 13 aa, Artificial Sequence
  Description of Artificial Sequence Synthetic peptide
QPFLMDLEGKQGN

SEQ ID NO: 4 - protein, 23 aa, Artificial Sequence
  Description of Artificial Sequence Synthetic peptide
TRFQTLLALHRSYLTPGDSSSGW

SEQ ID NO: 5 - protein, 18 aa, Artificial Sequence
  Description of Artificial Sequence Synthetic peptide
KSFTVEKGIYQTSNFRVQ

SEQ ID NO: 6 - protein, 17 aa, Artificial Sequence
  Description of Artificial Sequence Synthetic peptide
SASFSTFKCYGVSPTKL

SEQ ID NO: 7 - protein, 10 aa, Artificial Sequence
  Description of Artificial Sequence Synthetic peptide
KLPDDFTGCV

SEQ ID NO: 8 - protein, 18 aa, Artificial Sequence
  Description of Artificial Sequence Synthetic peptide
NLDSKVGGNYNYLYRLFR

SEQ ID NO: 9 - protein, 18 aa, Artificial Sequence
  Description of Artificial Sequence Synthetic peptide
YLYRLFRKSNLKPFERDI

SEQ ID NO: 10 - protein, 13 aa, Artificial Sequence
  Description of Artificial Sequence Synthetic peptide
KPFERDISTEIYQ

SEQ ID NO: 11 - protein, 18 aa, Artificial Sequence
  Description of Artificial Sequence Synthetic peptide
QSIIAYTMSLGAENSVAY

SEQ ID NO: 12 - protein, 9 aa, Artificial Sequence
  Description of Artificial Sequence Synthetic peptide
SIIAYTMSL

SEQ ID NO: 13 - protein, 17 aa, Artificial Sequence
  Description of Artificial Sequence Synthetic peptide
TECSNLLLQYGSFCTQL

SEQ ID NO: 14 - protein, 18 aa, Artificial Sequence
  Description of Artificial Sequence Synthetic peptide
VKQIYKTPPIKDFGGFNF

SEQ ID NO: 15 - protein, 17 aa, Artificial Sequence
  Description of Artificial Sequence Synthetic peptide
DSLSSTASALGKLQDVV

SEQ ID NO: 16 - protein, 9 aa, Artificial Sequence
  Description of Artificial Sequence Synthetic peptide
ALNTLVKQL

SEQ ID NO: 17 - protein, 9 aa, Artificial Sequence
  Description of Artificial Sequence Synthetic peptide
VLNDILSRL

SEQ ID NO: 18 - protein, 9 aa, Artificial Sequence
  Description of Artificial Sequence Synthetic peptide
LITGRLQSL

SEQ ID NO: 19 - protein, 18 aa, Artificial Sequence
  Description of Artificial Sequence Synthetic peptide
QLIRAAEIRASANLAATK

SEQ ID NO: 20 - protein, 15 aa, Artificial Sequence
  Description of Artificial Sequence Synthetic peptide
HWFVTQRNFYEPQII

SEQ ID NO: 21 - protein, 9 aa, Artificial Sequence
  Description of Artificial Sequence Synthetic peptide
RLNEVAKNL

SEQ ID NO: 22 - protein, 9 aa, Artificial Sequence
  Description of Artificial Sequence Synthetic peptide
NLNESLIDL

SEQ ID NO: 23 - protein, 9 aa, Artificial Sequence
  Description of Artificial Sequence Synthetic peptide
FIAGLIAIV

SEQ ID NO: 24 - protein, 15 aa, Artificial Sequence
  Description of Artificial Sequence Synthetic peptide
RIFTIGTVTLKQGEI

SEQ ID NO: 25 - protein, 9 aa, Artificial Sequence
  Description of Artificial Sequence Synthetic peptide
TVTLKQGEI

SEQ ID NO: 26 - protein, 10 aa, Artificial Sequence
  Description of Artificial Sequence Synthetic peptide
TLACFVLAAV

SEQ ID NO: 27 - protein, 9 aa, Artificial Sequence
  Description of Artificial Sequence Synthetic peptide
GLMWLSYFI

SEQ ID NO: 28 - protein, 9 aa, Artificial Sequence
  Description of Artificial Sequence Synthetic peptide
HLRIAGHHL

SEQ ID NO: 29 - protein, 9 aa, Artificial Sequence
  Description of Artificial Sequence Synthetic peptide
ALNTPKDHI

SEQ ID NO: 30 - protein, 9 aa, Artificial Sequence
  Description of Artificial Sequence Synthetic peptide
LQLPQGTTL

SEQ ID NO: 31 - protein, 10 aa, Artificial Sequence
  Description of Artificial Sequence Synthetic peptide
GDAALALLLL

SEQ ID NO: 32 - protein, 9 aa, Artificial Sequence
  Description of Artificial Sequence Synthetic peptide
LALLLLDRL

SEQ ID NO: 33 - protein, 9 aa, Artificial Sequence
  Description of Artificial Sequence Synthetic peptide
LLLDRLNQL

SEQ ID NO: 34 - protein, 9 aa, Artificial Sequence
  Description of Artificial Sequence Synthetic peptide
RLNQLESKM

SEQ ID NO: 35 - protein, 10 aa, Artificial Sequence
  Description of Artificial Sequence Synthetic peptide
TKAYNVTQAF

SEQ ID NO: 36 - protein, 9 aa, Artificial Sequence
  Description of Artificial Sequence Synthetic peptide
GMSRIGMEV

SEQ ID NO: 37 - protein, 10 aa, Artificial Sequence
  Description of Artificial Sequence Synthetic peptide
MEVTPSGTWL

SEQ ID NO: 38 - protein, 9 aa, Artificial Sequence
  Description of Artificial Sequence Synthetic peptide
NFKDQVILL

SEQ ID NO: 39 - protein, 9 aa, Artificial Sequence
  Description of Artificial Sequence Synthetic peptide
CLEASFNYL

SEQ ID NO: 40 - protein, 9 aa, Artificial Sequence
  Description of Artificial Sequence Synthetic peptide
WLMWLIINL

SEQ ID NO: 41 - protein, 9 aa, Artificial Sequence
  Description of Artificial Sequence Synthetic peptide
ILLLDQALV

SEQ ID NO: 42 - protein, 9 aa, Artificial Sequence
  Description of Artificial Sequence Synthetic peptide
SACVLAAEC

SEQ ID NO: 43 - protein, 9 aa, Artificial Sequence
  Description of Artificial Sequence Synthetic peptide
SLPGVFCGV

SEQ ID NO: 44 - protein, 9 aa, Artificial Sequence
  Description of Artificial Sequence Synthetic peptide
TLMNVLTLV

SEQ ID NO: 45 - protein, 9 aa, Artificial Sequence
  Description of Artificial Sequence Synthetic peptide
SMWALIISV

SEQ ID NO: 46 - protein, 20 aa, Artificial Sequence
  Description of Artificial Sequence Synthetic peptide
MFVFLVLLPLVSSQCVNLTT

SEQ ID NO: 47 - protein, 20 aa, Artificial Sequence
  Description of Artificial Sequence Synthetic peptide
VSSQCVNLTTRTQLPPAYTN

SEQ ID NO: 48 - protein, 20 aa, Artificial Sequence
  Description of Artificial Sequence Synthetic peptide
RTQLPPAYTNSFTRGVYYPD

SEQ ID NO: 49 - protein, 20 aa, Artificial Sequence
  Description of Artificial Sequence Synthetic peptide
SFTRGVYYPDKVFRSSVLHS

SEQ ID NO: 50 - protein, 20 aa, Artificial Sequence
  Description of Artificial Sequence Synthetic peptide
KVFRSSVLHSTQDLFLPFFS

SEQ ID NO: 51 - protein, 20 aa, Artificial Sequence
  Description of Artificial Sequence Synthetic peptide
TQDLFLPFFSNVTWFHAIHV

SEQ ID NO: 52 - protein, 20 aa, Artificial Sequence
  Description of Artificial Sequence Synthetic peptide
NVTWFHAIHVSGTNGTKRFD

SEQ ID NO: 53 - protein, 20 aa, Artificial Sequence
  Description of Artificial Sequence Synthetic peptide
SGTNGTKRFDNPVLPFNDGV

SEQ ID NO: 54 - protein, 20 aa, Artificial Sequence
  Description of Artificial Sequence Synthetic peptide
NPVLPFNDGVYFASTEKSNI

SEQ ID NO: 55 - protein, 20 aa, Artificial Sequence
  Description of Artificial Sequence Synthetic peptide
YFASTEKSNIIRGWIFGTTL

SEQ ID NO: 56 - protein, 20 aa, Artificial Sequence
  Description of Artificial Sequence Synthetic peptide
IRGWIFGTTLDSKTQSLLIV

SEQ ID NO: 57 - protein, 20 aa, Artificial Sequence
  Description of Artificial Sequence Synthetic peptide
DSKTQSLLIVNNATNVVIKV

SEQ ID NO: 58 - protein, 20 aa, Artificial Sequence
  Description of Artificial Sequence Synthetic peptide
NNATNVVIKVCEFQFCNDPF

SEQ ID NO: 59 - protein, 20 aa, Artificial Sequence
  Description of Artificial Sequence Synthetic peptide
CEFQFCNDPFLGVYYHKNNK

SEQ ID NO: 60 - protein, 20 aa, Artificial Sequence
  Description of Artificial Sequence Synthetic peptide
LGVYYHKNNKSWMESEFRVY

SEQ ID NO: 61 - protein, 20 aa, Artificial Sequence
  Description of Artificial Sequence Synthetic peptide
SWMESEFRVYSSANNCTFEY

SEQ ID NO: 62 - protein, 20 aa, Artificial Sequence
  Description of Artificial Sequence Synthetic peptide
SSANNCTFEYVSQPFLMDLE

SEQ ID NO: 63 - protein, 20 aa, Artificial Sequence
  Description of Artificial Sequence Synthetic peptide
VSQPFLMDLEGKQGNFKNLR

SEQ ID NO: 64 - protein, 20 aa, Artificial Sequence
  Description of Artificial Sequence Synthetic peptide
GKQGNFKNLREFVFKNIDGY

SEQ ID NO: 65 - protein, 20 aa, Artificial Sequence
  Description of Artificial Sequence Synthetic peptide
EFVFKNIDGYFKIYSKHTPI

SEQ ID NO: 66 - protein, 20 aa, Artificial Sequence
  Description of Artificial Sequence Synthetic peptide
FKIYSKHTPINLVRDLPQGF

SEQ ID NO: 67 - protein, 20 aa, Artificial Sequence
  Description of Artificial Sequence Synthetic peptide
NLVRDLPQGFSALEPLVDLP

SEQ ID NO: 68 - protein, 20 aa, Artificial Sequence
  Description of Artificial Sequence Synthetic peptide
SALEPLVDLPIGINITRFQT

SEQ ID NO: 69 - protein, 20 aa, Artificial Sequence
  Description of Artificial Sequence Synthetic peptide
IGINITRFQTLLALHRSYLT

SEQ ID NO: 70 - protein, 20 aa, Artificial Sequence
  Description of Artificial Sequence Synthetic peptide
LLALHRSYLTPGDSSSGWTA

SEQ ID NO: 71 - protein, 20 aa, Artificial Sequence
  Description of Artificial Sequence Synthetic peptide
PGDSSSGWTAGAAAYYVGYL

SEQ ID NO: 72 - protein, 20 aa, Artificial Sequence
  Description of Artificial Sequence Synthetic peptide
GAAAYYVGYLQPRTFLLKYN

SEQ ID NO: 73 - protein, 20 aa, Artificial Sequence
  Description of Artificial Sequence Synthetic peptide
QPRTFLLKYNENGTITDAVD

SEQ ID NO: 74 - protein, 20 aa, Artificial Sequence
  Description of Artificial Sequence Synthetic peptide
ENGTITDAVDCALDPLSETK

SEQ ID NO: 75 - protein, 20 aa, Artificial Sequence
  Description of Artifical Sequence Synthetic amino acid
CALDPLSETKCTLKSFTVEK

SEQ ID NO: 76 - protein, 20 aa, Artificial Sequence
  Description of Artificial Sequence Synthetic peptide
CTLKSFTVEKGIYQTSNFRV

SEQ ID NO: 77 - protein, 20 aa, Artificial Sequence
  Description of Artifical Sequence Synthetic amino acid
GIYQTSNFRVQPTESIVRFP

SEQ ID NO: 78 - protein, 20 aa, Artificial Sequence
  Description of Artificial Sequence Synthetic peptide
QPTESIVRFPNITNLCPFGE

SEQ ID NO: 79 - protein, 20 aa, Artificial Sequence
  Description of Artificial Sequence Synthetic peptide
NITNLCPFGEVFNATRFASV

SEQ ID NO: 80 - protein, 20 aa, Artificial Sequence
  Description of Artificial Sequence Synthetic peptide
VFNATRFASVYAWNRKRISN

SEQ ID NO: 81 - protein, 20 aa, Artificial Sequence
  Description of Artificial Sequence Synthetic peptide
YAWNRKRISNCVADYSVLYN

SEQ ID NO: 82 - protein, 20 aa, Artificial Sequence
  Description of Artificial Sequence Synthetic peptide
CVADYSVLYNSASFSTFKCY

SEQ ID NO: 83 - protein, 20 aa, Artificial Sequence
  Description of Artificial Sequence Synthetic peptide
SASFSTFKCYGVSPTKLNDL

SEQ ID NO: 84 - protein, 20 aa, Artificial Sequence
  Description of Artificial Sequence Synthetic peptide
GVSPTKLNDLCFTNVYADSF

SEQ ID NO: 85 - protein, 20 aa, Artificial Sequence
  Description of Artificial Sequence Synthetic peptide
CFTNVYADSFVIRGDEVRQI

SEQ ID NO: 86 - protein, 20 aa, Artificial Sequence
  Description of Artificial Sequence Synthetic peptide
VIRGDEVRQIAPGQTGKIAD

SEQ ID NO: 87 - protein, 20 aa, Artificial Sequence
  Description of Artificial Sequence Synthetic peptide
APGQTGKIADYNYKLPDDFT

SEQ ID NO: 88 - protein, 20 aa, Artificial Sequence
  Description of Artifical Sequence Synthetic amino acid
YNYKLPDDFTGCVIAWNSNN

SEQ ID NO: 89 - protein, 20 aa, Artificial Sequence
  Description of Artificial Sequence Synthetic peptide
GCVIAWNSNNLDSKVGGNYN

SEQ ID NO: 90 - protein, 20 aa, Artificial Sequence
  Description of Artificial Sequence Synthetic peptide
LDSKVGGNYNYLYRLFRKSN

SEQ ID NO: 91 - protein, 20 aa, Artificial Sequence
  Description of Artificial Sequence Synthetic peptide
YLYRLFRKSNLKPFERDIST

SEQ ID NO: 92 - protein, 19 aa, Artificial Sequence
  Description of Artificial Sequence Synthetic peptide
LKPFERDISTEIYQAGSTP

SEQ ID NO: 93 - protein, 20 aa, Artificial Sequence
  Description of Artificial Sequence Synthetic peptide
EIYQAGSTPCNGVEGFNCYF

SEQ ID NO: 94 - protein, 20 aa, Artificial Sequence
  Description of Artificial Sequence Synthetic peptide
NGVEGFNCYFPLQSYGFQPT

SEQ ID NO: 95 - protein, 20 aa, Artificial Sequence
  Description of Artifical Sequence Synthetic amino acid
PLQSYGFQPTNGVGYQPYRV

SEQ ID NO: 96 - protein, 20 aa, Artificial Sequence
  Description of Artificial Sequence Synthetic peptide
NGVGYQPYRVVVLSFELLHA

SEQ ID NO: 97 - protein, 20 aa, Artificial Sequence
  Description of Artificial Sequence Synthetic peptide
VVLSFELLHAPATVCGPKKS

SEQ ID NO: 98 - protein, 20 aa, Artificial Sequence
  Description of Artificial Sequence Synthetic peptide
PATVCGPKKSTNLVKNKCVN

SEQ ID NO: 99 - protein, 20 aa, Artificial Sequence
  Description of Artificial Sequence Synthetic peptide
TNLVKNKCVNFNFNGLTGTG

SEQ ID NO: 100 - protein, 20 aa, Artificial Sequence
  Description of Artificial Sequence Synthetic peptide
FNFNGLTGTGVLTESNKKFL

SEQ ID NO: 101 - protein, 20 aa, Artificial Sequence
  Description of Artificial Sequence Synthetic peptide
VLTESNKKFLPFQQFGRDIA

SEQ ID NO: 102 - protein, 20 aa, Artificial Sequence
  Description of Artificial Sequence Synthetic peptide
PFQQFGRDIADTTDAVRDPQ

SEQ ID NO: 103 - protein, 19 aa, Artificial Sequence
  Description of Artificial Sequence Synthetic peptide
DTTDAVRDPQTLEILDITP

SEQ ID NO: 104 - protein, 20 aa, Artificial Sequence
  Description of Artificial Sequence Synthetic peptide
TLEILDITPCSFGGVSVITP

SEQ ID NO: 105 - protein, 20 aa, Artificial Sequence
  Description of Artificial Sequence Synthetic peptide
SFGGVSVITPECDIPIGAGI

SEQ ID NO: 106 - protein, 20 aa, Artificial Sequence
  Description of Artificial Sequence Synthetic peptide
ECDIPIGAGICASYQTQTNS

SEQ ID NO: 107 - protein, 20 aa, Artificial Sequence
  Description of Artificial Sequence Synthetic peptide
CASYQTQTNSPRRARSVASQ

SEQ ID NO: 108 - protein, 20 aa, Artificial Sequence
  Description of Artificial Sequence Synthetic peptide
PRRARSVASQSIIAYTMSLG

SEQ ID NO: 109 - protein, 20 aa, Artificial Sequence
  Description of Artificial Sequence Synthetic peptide
SIIAYTMSLGAENSVAYSNN

SEQ ID NO: 110 - protein, 20 aa, Artificial Sequence
  Description of Artificial Sequence Synthetic peptide
AENSVAYSNNSIAIPTNFTI

SEQ ID NO: 111 - protein, 20 aa, Artificial Sequence
  Description of Artificial Sequence Synthetic peptide
SIAIPTNFTISVTTEILPVS

SEQ ID NO: 112 - protein, 20 aa, Artificial Sequence
  Description of Artificial Sequence Synthetic peptide
SVTTEILPVSMTKTSVDCTM

SEQ ID NO: 113 - protein, 20 aa, Artificial Sequence
  Description of Artificial Sequence Synthetic peptide
MTKTSVDCTMYICGDSTECS

SEQ ID NO: 114 - protein, 19 aa, Artificial Sequence
  Description of Artificial Sequence Synthetic peptide
YICGDSTECSNLLLQYGSF

SEQ ID NO: 115 - protein, 20 aa, Artificial Sequence
  Description of Artificial Sequence Synthetic peptide
NLLLQYGSFCTQLNRALTGI

SEQ ID NO: 116 - protein, 20 aa, Artificial Sequence
  Description of Artificial Sequence Synthetic peptide
TQLNRALTGIAVEQDKNTQE

SEQ ID NO: 117 - protein, 20 aa, Artificial Sequence
  Description of Artificial Sequence Synthetic peptide
AVEQDKNTQEVFAQVKQIYK

SEQ ID NO: 118 - protein, 20 aa, Artificial Sequence
  Description of Artificial Sequence Synthetic peptide
VFAQVKQIYKTPPIKDFGGF

SEQ ID NO: 119 - protein, 20 aa, Artificial Sequence
  Description of Artificial Sequence Synthetic peptide
TPPIKDFGGFNFSQILPDPS

SEQ ID NO: 120 - protein, 20 aa, Artificial Sequence
  Description of Artificial Sequence Synthetic peptide
NFSQILPDPSKPSKRSFIED

SEQ ID NO: 121 - protein, 20 aa, Artificial Sequence
  Description of Artificial Sequence Synthetic peptide
KPSKRSFIEDLLFNKVTLAD

SEQ ID NO: 122 - protein, 19 aa, Artificial Sequence
  Description of Artificial Sequence Synthetic peptide
LLFNKVTLADAGFIKQYGD

SEQ ID NO: 123 - protein, 20 aa, Artificial Sequence
  Description of Artificial Sequence Synthetic peptide
AGFIKQYGDCLGDIAARDLI

SEQ ID NO: 124 - protein, 20 aa, Artificial Sequence
  Description of Artificial Sequence Synthetic peptide
LGDIAARDLICAQKFNGLTV

SEQ ID NO: 125 - protein, 20 aa, Artificial Sequence
  Description of Artificial Sequence Synthetic peptide
CAQKFNGLTVLPPLLTDEMI

SEQ ID NO: 126 - protein, 20 aa, Artificial Sequence
  Description of Artificial Sequence Synthetic peptide
LPPLLTDEMIAQYTSALLAG

SEQ ID NO: 127 - protein, 20 aa, Artificial Sequence
  Description of Artificial Sequence Synthetic peptide
AQYTSALLAGTITSGWTFGA

SEQ ID NO: 128 - protein, 20 aa, Artificial Sequence
  Description of Artificial Sequence Synthetic peptide
TITSGWTFGAGAALQIPFAM

SEQ ID NO: 129 - protein, 20 aa, Artificial Sequence
  Description of Artificial Sequence Synthetic peptide
GAALQIPFAMQMAYRFNGIG

SEQ ID NO: 130 - protein, 20 aa, Artificial Sequence
  Description of Artificial Sequence Synthetic peptide
QMAYRFNGIGVTQNVLYENQ

SEQ ID NO: 131 - protein, 20 aa, Artificial Sequence
  Description of Artificial Sequence Synthetic peptide
VTQNVLYENQKLIANQFNSA

SEQ ID NO: 132 - protein, 20 aa, Artificial Sequence
  Description of Artificial Sequence Synthetic peptide
KLIANQFNSAIGKIQDSLSS

SEQ ID NO: 133 - protein, 20 aa, Artificial Sequence
  Description of Artificial Sequence Synthetic peptide
IGKIQDSLSSTASALGKLQD

SEQ ID NO: 134 - protein, 20 aa, Artificial Sequence
  Description of Artificial Sequence Synthetic peptide
TASALGKLQDVVNQNAQALN

SEQ ID NO: 135 - protein, 20 aa, Artificial Sequence
  Description of Artificial Sequence Synthetic peptide
VVNQNAQALNTLVKQLSSNF

SEQ ID NO: 136 - protein, 20 aa, Artificial Sequence
  Description of Artificial Sequence Synthetic peptide
TLVKQLSSNFGAISSVLNDI

SEQ ID NO: 137 - protein, 20 aa, Artificial Sequence
  Description of Artificial Sequence Synthetic peptide
GAISSVLNDILSRLDKVEAE

SEQ ID NO: 138 - protein, 20 aa, Artificial Sequence
  Description of Artificial Sequence Synthetic peptide
LSRLDKVEAEVQIDRLITGR

SEQ ID NO: 139 - protein, 20 aa, Artificial Sequence
  Description of Artificial Sequence Synthetic peptide
VQIDRLITGRLQSLQTYVTQ

SEQ ID NO: 140 - protein, 20 aa, Artificial Sequence
  Description of Artificial Sequence Synthetic amino acid
LQSLQTYVTQQLIRAAEIRA

SEQ ID NO: 141 - protein, 20 aa, Artificial Sequence
  Description of Artificial Sequence Synthetic peptide
QLIRAAEIRASANLAATKMS

SEQ ID NO: 142 - protein, 20 aa, Artificial Sequence
  Description of Artificial Sequence Synthetic peptide
SANLAATKMSECVLGQSKRV

SEQ ID NO: 143 - protein, 20 aa, Artificial Sequence
  Description of Artificial Sequence Synthetic peptide
ECVLGQSKRVDFCGKGYHLM

SEQ ID NO: 144 - protein, 20 aa, Artificial Sequence
  Description of Artificial Sequence Synthetic peptide
DFCGKGYHLMSFPQSAPHGV

SEQ ID NO: 145 - protein, 20 aa, Artificial Sequence
  Description of Artificial Sequence Synthetic peptide
SFPQSAPHGVVFLHVTYVPA

SEQ ID NO: 146 - protein, 20 aa, Artificial Sequence
  Description of Artificial Sequence Synthetic peptide
VFLHVTYVPAQEKNFTTAPA

SEQ ID NO: 147 - protein, 20 aa, Artificial Sequence
  Description of Artificial Sequence Synthetic peptide
QEKNFTTAPAICHDGKAHFP

SEQ ID NO: 148 - protein, 20 aa, Artificial Sequence
  Description of Artificial Sequence Synthetic peptide
ICHDGKAHFPREGVFVSNGT

SEQ ID NO: 149 - protein, 20 aa, Artificial Sequence
  Description of Artificial Sequence Synthetic peptide
REGVFVSNGTHWFVTQRNFY

SEQ ID NO: 150 - protein, 20 aa, Artificial Sequence
  Description of Artificial Sequence Synthetic peptide
HWFVTQRNFYEPQIITTDNT

SEQ ID NO: 151 - protein, 20 aa, Artificial Sequence
  Description of Artificial Sequence Synthetic peptide
EPQIITTDNTFVSGNCDVVI

SEQ ID NO: 152 - protein, 20 aa, Artificial Sequence
  Description of Artificial Sequence Synthetic peptide
FVSGNCDVVIGIVNNTVYDP

SEQ ID NO: 153 - protein, 20 aa, Artificial Sequence
  Description of Artificial Sequence Synthetic peptide
GIVNNTVYDPLQPELDSFKE

SEQ ID NO: 154 - protein, 20 aa, Artificial Sequence
  Description of Artificial Sequence Synthetic peptide
LQPELDSFKEELDKYFKNHT

SEQ ID NO: 155 - protein, 20 aa, Artificial Sequence
  Description of Artificial Sequence Synthetic peptide
ELDKYFKNHTSPDVDLGDIS

SEQ ID NO: 156 - protein, 20 aa, Artificial Sequence
  Description of Artificial Sequence Synthetic peptide
SPDVDLGDISGINASVVNIQ

SEQ ID NO: 157 - protein, 20 aa, Artificial Sequence
  Description of Artificial Sequence Synthetic peptide
GINASVVNIQKEIDRLNEVA

SEQ ID NO: 158 - protein, 20 aa, Artificial Sequence
  Description of Artificial Sequence Synthetic peptide
KEIDRLNEVAKNLNESLIDL

SEQ ID NO: 159 - protein, 20 aa, Artificial Sequence
  Description of Artificial Sequence Synthetic peptide
KNLNESLIDLQELGKYEQYI

SEQ ID NO: 160 - protein, 20 aa, Artificial Sequence
  Description of Artificial Sequence Synthetic peptide
QELGKYEQYIKWPWYIWLGF

SEQ ID NO: 161 - protein, 20 aa, Artificial Sequence
  Description of Artificial Sequence Synthetic peptide
KWPWYIWLGFIAGLIAIVMV

SEQ ID NO: 162 - protein, 19 aa, Artificial Sequence
  Description of Artificial Sequence Synthetic peptide
IAGLIAIVMVTIMLCCMTS

SEQ ID NO: 163 - protein, 19 aa, Artificial Sequence
  Description of Artificial Sequence Synthetic peptide
TIMLCCMTSCCSCLKGCCS

SEQ ID NO: 164 - protein, 20 aa, Artificial Sequence
  Description of Artificial Sequence Synthetic peptide
CSCLKGCCSCGSCCKFDEDD

SEQ ID NO: 165 - protein, 23 aa, Artificial Sequence
  Description of Artificial Sequence Synthetic peptide
GSCCKFDEDDSEPVLKGVKLHYT

SEQ ID NO: 166 - protein, 19 aa, Artificial Sequence
  Description of Artificial Sequence Synthetic peptide
SNQVAVLYQGVNCTEVPVA

SEQ ID NO: 167 - protein, 19 aa, Artificial Sequence
  Description of Artificial Sequence Synthetic peptide
IQDSLSSTAPALGKLQDVV

SEQ ID NO: 168 - protein, 20 aa, Artificial Sequence
  Description of Artificial Sequence Synthetic peptide
MAYCWRCTSCCFSERFQNHN

SEQ ID NO: 169 - protein, 20 aa, Artificial Sequence
  Description of Artificial Sequence Synthetic peptide
CFSERFQNHNHNPQKEMATS

SEQ ID NO: 170 - protein, 20 aa, Artificial Sequence
  Description of Artificial Sequence Synthetic peptide
PQKEMATSTLQGCSLCLQLA

SEQ ID NO: 171 - protein, 20 aa, Artificial Sequence
  Description of Artificial Sequence Synthetic peptide
TLQGCSLCLQLAVVVCNSLL

SEQ ID NO: 172 - protein, 17 aa, Artificial Sequence
  Description of Artificial Sequence Synthetic peptide
VVVCNSLLTPFARCCWP

SEQ ID NO: 173 - protein, 21 aa, Artificial Sequence
  Description of Artificial Sequence Synthetic peptide
MKFLVFLGIITTVAAFHQECS

SEQ ID NO: 174 - protein, 20 aa, Artificial Sequence
  Description of Artificial Sequence Synthetic peptide
TVAAFHQECSLQSCTQHQPY

SEQ ID NO: 175 - protein, 20 aa, Artificial Sequence
  Description of Artificial Sequence Synthetic peptide
LQSCTQHQPYVVDDPCPIHF

SEQ ID NO: 176 - protein, 20 aa, Artificial Sequence
  Description of Artificial Sequence Synthetic peptide
VVDDPCPIHFYSKWYIRVGA

SEQ ID NO: 177 - protein, 20 aa, Artificial Sequence
  Description of Artificial Sequence Synthetic peptide
YSKWYIRVGARKSAPLIELC

SEQ ID NO: 178 - protein, 20 aa, Artificial Sequence
  Description of Artificial Sequence Synthetic peptide
RKSAPLIELCVDEAGSKSPI

SEQ ID NO: 179 - protein, 20 aa, Artificial Sequence
  Description of Artificial Sequence Synthetic peptide
VDEAGSKSPIQYIDIGNYTV

SEQ ID NO: 180 - protein, 20 aa, Artificial Sequence
  Description of Artificial Sequence Synthetic peptide
QYIDIGNYTVSCLPFTINCQ

SEQ ID NO: 181 - protein, 20 aa, Artificial Sequence
  Description of Artificial Sequence Synthetic peptide
SCLPFTINCQEPKLGSLVVR

SEQ ID NO: 182 - protein, 20 aa, Artificial Sequence
  Description of Artificial Sequence Synthetic peptide
EPKLGSLVVRCSFYEDFLEY

SEQ ID NO: 183 - protein, 20 aa, Artificial Sequence
  Description of Artificial Sequence Synthetic peptide
CSFYEDFLEYHDVRVVLDFI

SEQ ID NO: 184 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 185 - protein, 57 aa, Artificial Sequence
  Description of Artificial Sequence Synthetic peptide
MAYCWRCTSCCFSERFQNHNPQKEMATSTLQGCSLCLQLAVVVCNSLLTPFARCCWP

SEQ ID NO: 186 - protein, 121 aa, Severe acute respiratory syndrome coronavirus 2
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 187 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 188 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 189 - protein, 20 aa, Artificial Sequence
  Description of Artificial Sequence Synthetic peptide
QYIDIGNYTVSCSPFTINCQ

SEQ ID NO: 190 - protein, 20 aa, Artificial Sequence
  Description of Artificial Sequence Synthetic peptide
SCSPFTINCQEPKLGSLVVR

SEQ ID NO: 191 - protein, 20 aa, Artificial Sequence
  Description of Artificial Sequence Synthetic peptide
MGYINVFAFPFTIYSLLLCR

SEQ ID NO: 192 - protein, 20 aa, Artificial Sequence
  Description of Artificial Sequence Synthetic peptide
FTIYSLLLCRMNSRNYIAQV

SEQ ID NO: 193 - protein, 20 aa, Artificial Sequence
  Description of Artificial Sequence Synthetic peptide
CRMNSRNYIAQVDVVNVNLT

SEQ ID NO: 194 - protein, 20 aa, Artificial Sequence
  Description of Artificial Sequence Synthetic peptide
VVLLSVLQQLRVESSSKLWA

SEQ ID NO: 195 - protein, 20 aa, Artificial Sequence
  Description of Artificial Sequence Synthetic peptide
RVESSSKLWAQCVQLHNDIL

SEQ ID NO: 196 - protein, 20 aa, Artificial Sequence
  Description of Artificial Sequence Synthetic peptide
QCVQLHNDILLAKDTTEAFE

SEQ ID NO: 197 - protein, 20 aa, Artificial Sequence
  Description of Artificial Sequence Synthetic peptide
LAKDTTEAFEKMVSLLSVLL

SEQ ID NO: 198 - protein, 20 aa, Artificial Sequence
  Description of Artificial Sequence Synthetic peptide
KMVSLLSVLLSMQGAVDINK

SEQ ID NO: 199 - protein, 20 aa, Artificial Sequence
  Description of Artificial Sequence Synthetic peptide
SMQGAVDINKLCEEMLDNRA

SEQ ID NO: 200 - protein, 20 aa, Artificial Sequence
  Description of Artificial Sequence Synthetic peptide
LCEEMLDNRATLQAIASEFS

SEQ ID NO: 201 - protein, 20 aa, Artificial Sequence
  Description of Artificial Sequence Synthetic peptide
TLQAIASEFSSLPSYAAFAT

SEQ ID NO: 202 - protein, 20 aa, Artificial Sequence
  Description of Artificial Sequence Synthetic peptide
SLPSYAAFATAQEAYEQAVA

SEQ ID NO: 203 - protein, 20 aa, Artificial Sequence
  Description of Artificial Sequence Synthetic peptide
AQEAYEQAVANGDSEVVLKK

SEQ ID NO: 204 - protein, 20 aa, Artificial Sequence
  Description of Artificial Sequence Synthetic peptide
NGDSEVVLKKLKKSLNVAKS

SEQ ID NO: 205 - protein, 20 aa, Artificial Sequence
  Description of Artificial Sequence Synthetic peptide
LKKSLNVAKSEFDRDAAMQR

SEQ ID NO: 206 - protein, 20 aa, Artificial Sequence
  Description of Artificial Sequence Synthetic peptide
EFDRDAAMQRKLEKMADQAM

SEQ ID NO: 207 - protein, 20 aa, Artificial Sequence
  Description of Artificial Sequence Synthetic peptide
KLEKMADQAMTQMYKQARSE

SEQ ID NO: 208 - protein, 20 aa, Artificial Sequence
  Description of Artificial Sequence Synthetic peptide
TQMYKQARSEDKRAKVTSAM

SEQ ID NO: 209 - protein, 20 aa, Artificial Sequence
  Description of Artificial Sequence Synthetic peptide
DKRAKVTSAMQTMLFTMLRK

SEQ ID NO: 210 - protein, 20 aa, Artificial Sequence
  Description of Artificial Sequence Synthetic peptide
QTMLFTMLRKLDNDALNNII

SEQ ID NO: 211 - protein, 20 aa, Artificial Sequence
  Description of Artificial Sequence Synthetic peptide
LDNDALNNIINNARDGCVPL

SEQ ID NO: 212 - protein, 20 aa, Artificial Sequence
  Description of Artificial Sequence Synthetic peptide
NNARDGCVPLNIIPLTTAAK

SEQ ID NO: 213 - protein, 20 aa, Artificial Sequence
  Description of Artificial Sequence Synthetic peptide
NIIPLTTAAKLMVVIPDYNT

SEQ ID NO: 214 - protein, 20 aa, Artificial Sequence
  Description of Artificial Sequence Synthetic peptide
LMVVIPDYNTYKNTCDGTTF

SEQ ID NO: 215 - protein, 20 aa, Artificial Sequence
  Description of Artificial Sequence Synthetic peptide
YKNTCDGTTFTYASALWEIQ

SEQ ID NO: 216 - protein, 20 aa, Artificial Sequence
  Description of Artificial Sequence Synthetic peptide
TYASALWEIQQVVDADSKIV

SEQ ID NO: 217 - protein, 20 aa, Artificial Sequence
  Description of Artificial Sequence Synthetic peptide
QVVDADSKIVQLSEISMDNS

SEQ ID NO: 218 - protein, 20 aa, Artificial Sequence
  Description of Artificial Sequence Synthetic peptide
QLSEISMDNSPNLAWPLIVT

SEQ ID NO: 219 - protein, 20 aa, Artificial Sequence
  Description of Artificial Sequence Synthetic peptide
PNLAWPLIVTALRANSAVKL

SEQ ID NO: 220 - protein, 20 aa, Artificial Sequence
  Description of Artificial Sequence Synthetic peptide
ALRANSAVKLQNNELSPVAL

SEQ ID NO: 221 - protein, 20 aa, Artificial Sequence
  Description of Artificial Sequence Synthetic peptide
QNNELSPVALRQMSCAAGTT

SEQ ID NO: 222 - protein, 20 aa, Artificial Sequence
  Description of Artificial Sequence Synthetic peptide
RQMSCAAGTTQTACTDDNAL

SEQ ID NO: 223 - protein, 20 aa, Artificial Sequence
  Description of Artificial Sequence Synthetic peptide
QTACTDDNALAYYNTTKGGR

SEQ ID NO: 224 - protein, 20 aa, Artificial Sequence
  Description of Artificial Sequence Synthetic peptide
AYYNTTKGGRFVLALLSDLQ

SEQ ID NO: 225 - protein, 20 aa, Artificial Sequence
  Description of Artificial Sequence Synthetic peptide
FVLALLSDLQDLKWARFPKS

SEQ ID NO: 226 - protein, 20 aa, Artificial Sequence
  Description of Artificial Sequence Synthetic peptide
DLKWARFPKSDGTGTIYTEL

SEQ ID NO: 227 - protein, 20 aa, Artificial Sequence
  Description of Artificial Sequence Synthetic peptide
DGTGTIYTELEPPCRFVTDT

SEQ ID NO: 228 - protein, 20 aa, Artificial Sequence
  Description of Artificial Sequence Synthetic peptide
EPPCRFVTDTPKGPKVKYLY

SEQ ID NO: 229 - protein, 20 aa, Artificial Sequence
  Description of Artificial Sequence Synthetic peptide
PKGPKVKYLYFIKGLNNLNR

SEQ ID NO: 230 - protein, 354 aa, Severe acute respiratory syndrome coronavirus 2
KLWAQCVQLHNDILLAKDTTEAFEKMVSLLSVLLSMQGAVDINKLCEEMLDNRATLQAIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQNNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNR

SEQ ID NO: 231 - protein, 370 aa, Artificial Sequence
  Description of Artificial Sequence Synthetic peptide
VVLLSVLQQLRVESSSKLWAQCVQLHNDILLAKDTTEAFEKMVSLLSVLLSMQGAVDINKLCEEMLDNRATLQAIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQNNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNR

SEQ ID NO: 232 - protein, 38 aa, Severe acute respiratory syndrome coronavirus 2
MGYINVFAFPFTIYSLLLCRMNSRNYIAQVDVVNVNLT

SEQ ID NO: 233 - protein, 275 aa, Severe acute respiratory syndrome coronavirus 2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 234 - protein, 1531 aa, Severe acute respiratory syndrome coronavirus 2
CLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGGKIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQSAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISV

SEQ ID NO: 235 - protein, 20 aa, Artificial Sequence
  Description of Artificial Sequence Synthetic peptide
VSSQCVNFTTRTQLPPAYTN

SEQ ID NO: 236 - protein, 20 aa, Artificial Sequence
  Description of Artificial Sequence Synthetic peptide
VSSQCVNFTNRTQLPSAYTN

SEQ ID NO: 237 - protein, 20 aa, Artificial Sequence
  Description of Artificial Sequence Synthetic peptide
RTQLPSAYTNSFTRGVYYPD

SEQ ID NO: 238 - protein, 18 aa, Artificial Sequence
  Description of Artificial Sequence Synthetic peptide
TQDLFLPFFSNVTWFHAI

SEQ ID NO: 239 - protein, 18 aa, Artificial Sequence
  Description of Artificial Sequence Synthetic peptide
NVTWFHAISGTNGTKRFD

SEQ ID NO: 240 - protein, 20 aa, Artificial Sequence
  Description of Artificial Sequence Synthetic peptide
NVTWFHAIHVSGTNGTKRFA

SEQ ID NO: 241 - protein, 20 aa, Artificial Sequence
  Description of Artificial Sequence Synthetic peptide
SGTNGTKRFANPVLPFNDGV

SEQ ID NO: 242 - protein, 20 aa, Artificial Sequence
  Description of Artificial Sequence Synthetic peptide
CEFQFCNYPFLGVYYHKNNK

SEQ ID NO: 243 - protein, 19 aa, Artificial Sequence
  Description of Artificial Sequence Synthetic peptide
CEFQFCNDPFLGVYHKNNK

SEQ ID NO: 244 - protein, 19 aa, Artificial Sequence
  Description of Artificial Sequence Synthetic peptide
LGVYHKNNKSWMESEFRVY

SEQ ID NO: 245 - protein, 20 aa, Artificial Sequence
  Description of Artificial Sequence Synthetic peptide
VSQPFLMDLEGKQGNFKNLS

SEQ ID NO: 246 - protein, 20 aa, Artificial Sequence
  Description of Artificial Sequence Synthetic peptide
GKQGNFKNLSEFVFKNIDGY

SEQ ID NO: 247 - protein, 20 aa, Artificial Sequence
  Description of Artificial Sequence Synthetic peptide
FKIYSKHTPINLVRGLPQGF

SEQ ID NO: 248 - protein, 20 aa, Artificial Sequence
  Description of Artificial Sequence Synthetic peptide
NLVRGLPQGFSALEPLVDLP

SEQ ID NO: 249 - protein, 17 aa, Artificial Sequence
  Description of Artificial Sequence Synthetic peptide
IGINITRFQTLHISYLT

SEQ ID NO: 250 - protein, 17 aa, Artificial Sequence
  Description of Artificial Sequence Synthetic peptide
LHISYLTPGDSSSGWTA

SEQ ID NO: 251 - protein, 20 aa, Artificial Sequence
  Description of Artificial Sequence Synthetic peptide
VIRGDEVRQIAPGQTGNIAD

SEQ ID NO: 252 - protein, 20 aa, Artificial Sequence
  Description of Artificial Sequence Synthetic peptide
VIRGDEVRQIAPGQTGTIAD

SEQ ID NO: 253 - protein, 20 aa, Artificial Sequence
  Description of Artificial Sequence Synthetic peptide
APGQTGNIADYNYKLPDDFT

SEQ ID NO: 254 - protein, 20 aa, Artificial Sequence
  Description of Artificial Sequence Synthetic peptide
APGQTGTIADYNYKLPDDFT

SEQ ID NO: 255 - protein, 20 aa, Artificial Sequence
  Description of Artificial Sequence Synthetic peptide
EIYQAGSTPCNGVKGFNCYF

SEQ ID NO: 256 - protein, 20 aa, Artificial Sequence
  Description of Artificial Sequence Synthetic peptide
NGVKGFNCYFPLQSYGFQPT

SEQ ID NO: 257 - protein, 20 aa, Artificial Sequence
  Description of Artificial Sequence Synthetic peptide
GFNCYFPLQSYGFQPTYGVG

SEQ ID NO: 258 - protein, 20 aa, Artificial Sequence
  Description of Artificial Sequence Synthetic peptide
PLQSYGFQPTYGVGYQPYRV

SEQ ID NO: 259 - protein, 20 aa, Artificial Sequence
  Description of Artificial Sequence Synthetic peptide
YGVGYQPYRVVVLSFELLHA

SEQ ID NO: 260 - protein, 20 aa, Artificial Sequence
  Description of Artificial Sequence Synthetic peptide
VLTESNKKFLPFQQFGRDID

SEQ ID NO: 261 - protein, 20 aa, Artificial Sequence
  Description of Artificial Sequence Synthetic peptide
PFQQFGRDIDDTTDAVRDPQ

SEQ ID NO: 262 - protein, 20 aa, Artificial Sequence
  Description of Artificial Sequence Synthetic peptide
SFGGVSVITPGTNTSNQVAV

SEQ ID NO: 263 - protein, 20 aa, Artificial Sequence
  Description of Artificial Sequence Synthetic peptide
ITPGTNTSNQVAVLYQDVNC

SEQ ID NO: 264 - protein, 20 aa, Artificial Sequence
  Description of Artificial Sequence Synthetic peptide
ITPGTNTSNQVAVLYQGVNC

SEQ ID NO: 265 - protein, 20 aa, Artificial Sequence
  Description of Artificial Sequence Synthetic peptide
GTNTSNQVAVLYQDVNCTEV

SEQ ID NO: 266 - protein, 20 aa, Artificial Sequence
  Description of Artificial Sequence Synthetic peptide
GTNTSNQVAVLYQGVNCTEV

SEQ ID NO: 267 - protein, 20 aa, Artificial Sequence
  Description of Artificial Sequence Synthetic peptide
LYQDVNCTEVPVAIHADQLT

SEQ ID NO: 268 - protein, 20 aa, Artificial Sequence
  Description of Artificial Sequence Synthetic peptide
LYQGVNCTEVPVAIHADQLT

SEQ ID NO: 269 - protein, 20 aa, Artificial Sequence
  Description of Artificial Sequence Synthetic peptide
PVAIHADQLTPTWRVYSTGS

SEQ ID NO: 270 - protein, 20 aa, Artificial Sequence
  Description of Artificial Sequence Synthetic peptide
PTWRVYSTGSNVFQTRAGCL

SEQ ID NO: 271 - protein, 20 aa, Artificial Sequence
  Description of Artificial Sequence Synthetic peptide
NVFQTRAGCLIGAEHVNNSY

SEQ ID NO: 272 - protein, 20 aa, Artificial Sequence
  Description of Artificial Sequence Synthetic peptide
NVFQTRAGCLIGAEYVNNSY

SEQ ID NO: 273 - protein, 20 aa, Artificial Sequence
  Description of Artificial Sequence Synthetic peptide
IGAEHVNNSYECDIPIGAGI

SEQ ID NO: 274 - protein, 20 aa, Artificial Sequence
  Description of Artificial Sequence Synthetic peptide
IGAEYVNNSYECDIPIGAGI

SEQ ID NO: 275 - protein, 20 aa, Artificial Sequence
  Description of Artificial Sequence Synthetic peptide
SIIAYTMSLGVENSVAYSNN

SEQ ID NO: 276 - protein, 20 aa, Artificial Sequence
  Description of Artificial Sequence Synthetic peptide
VENSVAYSNNSIAIPTNFTI

SEQ ID NO: 277 - protein, 20 aa, Artificial Sequence
  Description of Artificial Sequence Synthetic peptide
AENSVAYSNNSIAIPINFTI

SEQ ID NO: 278 - protein, 20 aa, Artificial Sequence
  Description of Artificial Sequence Synthetic peptide
GAISSVLNDILARLDKVEAE

SEQ ID NO: 279 - protein, 20 aa, Artificial Sequence
  Description of Artificial Sequence Synthetic peptide
LARLDKVEAEVQIDRLITGR

SEQ ID NO: 280 - protein, 20 aa, Artificial Sequence
  Description of Artificial Sequence Synthetic peptide
SANLAAIKMSECVLGQSKRV

SEQ ID NO: 281 - protein, 20 aa, Artificial Sequence
  Description of Artificial Sequence Synthetic peptide
HWFVTQRNFYEPQIITTHNT

SEQ ID NO: 282 - protein, 9 aa, Artificial Sequence
  Description of Artificial Sequence Synthetic peptide
PQIITTHNT

SEQ ID NO: 283 - protein, 20 aa, Artificial Sequence
  Description of Artificial Sequence Synthetic peptide
EPQIITTHNTFVSGNCDVVI

SEQ ID NO: 284 - protein, 20 aa, Artificial Sequence
  Description of Artificial Sequence Synthetic peptide
LGIITTVAAFHQECSLQSCT

SEQ ID NO: 285 - protein, 20 aa, Artificial Sequence
  Description of Artificial Sequence Synthetic peptide
YSKWYIRVGAIKSAPLIELC

SEQ ID NO: 286 - protein, 20 aa, Artificial Sequence
  Description of Artificial Sequence Synthetic peptide
VDEAGSKSPIQSIDIGNYTV

SEQ ID NO: 287 - protein, 20 aa, Artificial Sequence
  Description of Artificial Sequence Synthetic peptide
VDEAGSKSPIQCIDIGNYTV

SEQ ID NO: 288 - protein, 20 aa, Artificial Sequence
  Description of Artificial Sequence Synthetic peptide
QSIDIGNYTVSCLPFTINCQ

SEQ ID NO: 289 - protein, 20 aa, Artificial Sequence
  Description of Artificial Sequence Synthetic peptide
QCIDIGNYTVSCLPFTINCQ

SEQ ID NO: 290 - protein, 20 aa, Artificial Sequence
  Description of Artificial Sequence Synthetic peptide
VSIQCVNLTTRTQLPPAYTN

SEQ ID NO: 291 - protein, 20 aa, Artificial Sequence
  Description of Artificial Sequence Synthetic peptide
LGVYYHKNNKSCMESEFRVY

SEQ ID NO: 292 - protein, 16 aa, Artificial Sequence
  Description of Artificial Sequence Synthetic peptide
DSLSSTASALGKLQDW

SEQ ID NO: 293 - protein, 20 aa, Artificial Sequence
  Description of Artificial Sequence Synthetic peptide
VSSQCVNLRTRTQLPPAYTN

SEQ ID NO: 294 - protein, 18 aa, Artificial Sequence
  Description of Artificial Sequence Synthetic peptide
SFTRGVYYPDKVFRSSVL

SEQ ID NO: 295 - protein, 20 aa, Artificial Sequence
  Description of Artificial Sequence Synthetic peptide
TQDLFLPFFSNVTWFHAIHF

SEQ ID NO: 296 - protein, 20 aa, Artificial Sequence
  Description of Artificial Sequence Synthetic peptide
NVTWFHAIHFSGTNGTKRFD

SEQ ID NO: 297 - protein, 20 aa, Artificial Sequence
  Description of Artificial Sequence Synthetic peptide
NPVLPFNDGVYFASIEKSNI

SEQ ID NO: 298 - protein, 20 aa, Artificial Sequence
  Description of Artificial Sequence Synthetic peptide
YFASIEKSNIIRGWIFGTTL

SEQ ID NO: 299 - protein, 9 aa, Artificial Sequence
  Description of Artificial Sequence Synthetic peptide
IFGTTLDSK

SEQ ID NO: 300 - protein, 10 aa, Artificial Sequence
  Description of Artificial Sequence Synthetic peptide
DSKTQSLLIV

SEQ ID NO: 301 - protein, 20 aa, Artificial Sequence
  Description of Artificial Sequence Synthetic peptide
CEFQFCNDPFLDVYYHKNNK

SEQ ID NO: 302 - protein, 18 aa, Artificial Sequence
  Description of Artificial Sequence Synthetic peptide
LDVYYHKNNKSWMESGVY

SEQ ID NO: 303 - protein, 19 aa, Artificial Sequence
  Description of Artificial Sequence Synthetic peptide
FVFKNIDGYFKIYSKHTPI

SEQ ID NO: 304 - protein, 20 aa, Artificial Sequence
  Description of Artificial Sequence Synthetic peptide
NLVRDLPQGFSVLEPLVDLP

SEQ ID NO: 305 - protein, 20 aa, Artificial Sequence
  Description of Artificial Sequence Synthetic peptide
SVLEPLVDLPIGINITRFQT

SEQ ID NO: 306 - protein, 20 aa, Artificial Sequence
  Description of Artificial Sequence Synthetic peptide
LLALHRSYLTPGDSSSGLTA

SEQ ID NO: 307 - protein, 19 aa, Artificial Sequence
  Description of Artificial Sequence Synthetic peptide
GDSSSGWTAGAAAYYVGYL

SEQ ID NO: 308 - protein, 17 aa, Artificial Sequence
  Description of Artificial Sequence Synthetic peptide
RTFLLKYNENGTITDAV

SEQ ID NO: 309 - protein, 18 aa, Artificial Sequence
  Description of Artificial Sequence Synthetic peptide
PTESIVRFPNITNLCPFG

SEQ ID NO: 310 - protein, 17 aa, Artificial Sequence
  Description of Artificial Sequence Synthetic peptide
VADYSVLYNSASFSTFK

SEQ ID NO: 311 - protein, 19 aa, Artificial Sequence
  Description of Artificial Sequence Synthetic peptide
FTNVYADSFVIRGDEVRQI

SEQ ID NO: 312 - protein, 18 aa, Artificial Sequence
  Description of Artificial Sequence Synthetic peptide
YNYKLPDDFTGCVIAWNS

SEQ ID NO: 313 - protein, 20 aa, Artificial Sequence
  Description of Artificial Sequence Synthetic peptide
LDSKVGGNYNYRYRLFRKSN

SEQ ID NO: 314 - protein, 20 aa, Artificial Sequence
  Description of Artificial Sequence Synthetic peptide
YRYRLFRKSNLKPFERDIST

SEQ ID NO: 315 - protein, 19 aa, Artificial Sequence
  Description of Artificial Sequence Synthetic peptide
LKPFERDISTEIYQAGSKP

SEQ ID NO: 316 - protein, 20 aa, Artificial Sequence
  Description of Artificial Sequence Synthetic peptide
EIYQAGSKPCNGVEGFNCYF

SEQ ID NO: 317 - protein, 19 aa, Artificial Sequence
  Description of Artificial Sequence Synthetic peptide
LQSYGFQPTNGVGYQPYRV

SEQ ID NO: 318 - protein, 19 aa, Artificial Sequence
  Description of Artificial Sequence Synthetic peptide
WLSFELLHAPATVCGPKKS

SEQ ID NO: 319 - protein, 19 aa, Artificial Sequence
  Description of Artificial Sequence Synthetic peptide
ATVCGPKKSTNLVKNKCVN

SEQ ID NO: 320 - protein, 19 aa, Artificial Sequence
  Description of Artificial Sequence Synthetic peptide
FQQFGRDIADTTDAVRDPQ

SEQ ID NO: 321 - protein, 18 aa, Artificial Sequence
  Description of Artificial Sequence Synthetic peptide
TLEILDITPSSFGGVSVI

SEQ ID NO: 322 - protein, 20 aa, Artificial Sequence
  Description of Artificial Sequence Synthetic peptide
CASYQTQTNSRRRARSVASQ

SEQ ID NO: 323 - protein, 19 aa, Artificial Sequence
  Description of Artificial Sequence Synthetic peptide
RRARSVASQSIIAYTMSLG

SEQ ID NO: 324 - protein, 19 aa, Artificial Sequence
  Description of Artificial Sequence Synthetic peptide
SVTTEILPVSMTKTSVDCT

SEQ ID NO: 325 - protein, 10 aa, Artificial Sequence
  Description of Artificial Sequence Synthetic peptide
ALLAGTITSG

SEQ ID NO: 326 - protein, 19 aa, Artificial Sequence
  Description of Artificial Sequence Synthetic peptide
TITSGWTFGAGAALQIPFA

SEQ ID NO: 327 - protein, 19 aa, Artificial Sequence
  Description of Artificial Sequence Synthetic peptide
TASALGKLQDVVNQNAQAL

SEQ ID NO: 328 - protein, 20 aa, Artificial Sequence
  Description of Artificial Sequence Synthetic peptide
TASALGKLQNVVNQNAQALN

SEQ ID NO: 329 - protein, 19 aa, Artificial Sequence
  Description of Artificial Sequence Synthetic peptide
TASALGKLQNVVNQNAQAL

SEQ ID NO: 330 - protein, 19 aa, Artificial Sequence
  Description of Artificial Sequence Synthetic peptide
TASALGKLQDWNQNAQALN

SEQ ID NO: 331 - protein, 18 aa, Artificial Sequence
  Description of Artificial Sequence Synthetic peptide
TASALGKLQDWNQNAQAL

SEQ ID NO: 332 - protein, 19 aa, Artificial Sequence
  Description of Artificial Sequence Synthetic peptide
WNQNAQALNTLVKQLSSNF

SEQ ID NO: 333 - protein, 19 aa, Artificial Sequence
  Description of Artificial Sequence Synthetic peptide
ECVLGQSKRVDFCGKGYHL

SEQ ID NO: 334 - protein, 19 aa, Artificial Sequence
  Description of Artificial Sequence Synthetic peptide
SFPQSAPHGWFLHVTYVPA

SEQ ID NO: 335 - protein, 19 aa, Artificial Sequence
  Description of Artificial Sequence Synthetic peptide
WFVTQRNFYEPQIITTDNT

SEQ ID NO: 336 - protein, 10 aa, Artificial Sequence
  Description of Artificial Sequence Synthetic peptide
TQRNFYEPQI

SEQ ID NO: 337 - protein, 9 aa, Artificial Sequence
  Description of Artificial Sequence Synthetic peptide
PQIITTDNT

SEQ ID NO: 338 - protein, 10 aa, Artificial Sequence
  Description of Artificial Sequence Synthetic peptide
TQRNFVEPQI

SEQ ID NO: 339 - protein, 19 aa, Artificial Sequence
  Description of Artificial Sequence Synthetic peptide
SPDVDLGDISGINASWNIQ

SEQ ID NO: 340 - protein, 19 aa, Artificial Sequence
  Description of Artificial Sequence Synthetic peptide
GINASWNIQKEIDRLNEVA

SEQ ID NO: 341 - protein, 22 aa, Artificial Sequence
  Description of Artificial Sequence Synthetic peptide
MAYCWRCTSCCFSERFQNHNPQ

SEQ ID NO: 342 - protein, 20 aa, Artificial Sequence
  Description of Artificial Sequence Synthetic peptide
CFSERFQNHNPQKEMATSTL

SEQ ID NO: 343 - protein, 16 aa, Artificial Sequence
  Description of Artificial Sequence Synthetic peptide
VWCNSLLTPFARCCWP

SEQ ID NO: 344 - protein, 19 aa, Artificial Sequence
  Description of Artificial Sequence Synthetic peptide
TVMFHQECSLQSCTQHQPY

SEQ ID NO: 345 - protein, 18 aa, Artificial Sequence
  Description of Artificial Sequence Synthetic peptide
SKWYIRVGARKSAPLIEL

SEQ ID NO: 346 - protein, 15 aa, Artificial Sequence
  Description of Artificial Sequence Synthetic peptide
IDIGNYTVSCLPFTI

SEQ ID NO: 347 - protein, 12 aa, Artificial Sequence
  Description of Artificial Sequence Synthetic peptide
GNYTVSCSPFTI

SEQ ID NO: 348 - protein, 12 aa, Artificial Sequence
  Description of Artificial Sequence Synthetic peptide
SQEPKLGSLVVR

SEQ ID NO: 349 - protein, 13 aa, Artificial Sequence
  Description of Artificial Sequence Synthetic peptide
RCSFYEDFLEYED

SEQ ID NO: 350 - protein, 15 aa, Artificial Sequence
  Description of Artificial Sequence Synthetic peptide
FYEDFLEYHDVRVVL

SEQ ID NO: 351 - protein, 16 aa, Artificial Sequence
  Description of Artificial Sequence Synthetic peptide
RFQTLHISYLTPGDSS

SEQ ID NO: 352 - protein, 16 aa, Artificial Sequence
  Description of Artificial Sequence Synthetic peptide
GVKGFNSYFPLQSYGF

SEQ ID NO: 353 - protein, 15 aa, Artificial Sequence
  Description of Artificial Sequence Synthetic peptide
GTNTSNQVAVLYQGV

SEQ ID NO: 354 - protein, 19 aa, Artificial Sequence
  Description of Artificial Sequence Synthetic peptide
LYQDVNSTEVPVAIHADQL

SEQ ID NO: 355 - protein, 19 aa, Artificial Sequence
  Description of Artificial Sequence Synthetic peptide
VAIHADQLTPTWRVYSTGS

SEQ ID NO: 356 - protein, 19 aa, Artificial Sequence
  Description of Artificial Sequence Synthetic peptide
TWRVYSTGSNVFQTRAGSL

SEQ ID NO: 357 - protein, 20 aa, Artificial Sequence
  Description of Artificial Sequence Synthetic peptide
NVFQTRAGSLIGAEHVNNSY

SEQ ID NO: 358 - protein, 14 aa, Artificial Sequence
  Description of Artificial Sequence Synthetic peptide
VENSVAYSNNSIAI

(19) United States
(12) Patent Application Publication
(10) Pub. No.: US 2022/0016268 A1
(43) Pub. Date: Jan. 20, 2022

(54) SKIN-BASED TESTING FOR DETECTION OF CELL-MEDIATED IMMUNE RESPONSES TO SARS-COV-2

(71) Applicants: TONIX PHARMACEUTICALS HOLDING CORP., Chatham, NJ (US); TONIX PHARMA LIMITED, Dublin 2 (IE)

(72) Inventors: Seth Lederman, South Dartmouth, MA (US); Siobhan J. Fogarty, Dublin (IE)

(21) Appl. No.: 17/378,642

(22) Filed: Jul. 16, 2021

Related U.S. Application Data
(60) Provisional application No. 63/147,235, filed on Feb. 8, 2021.
(60) Provisional application No. 63/143,021, filed on Jan. 28, 2021.
(60) Provisional application No. 63/053,484, filed on Jul. 17, 2020.

(51) Int. Cl. A61K 49/00; C07K 14/005

(52) U.S. Cl. CPC A61K 49/0006; C12N 2770/18022; C07K 14/005

(57) ABSTRACT

The present disclosure provides methods for detecting cell-mediated immunity to SARS-CoV-2 in a subject, methods for detecting a cell-mediated immune response against SARS-CoV-2 in a subject and methods for determining if a vaccine against SARS-CoV-2 elicits a cell-mediated immune response in a subject, comprising administering to the skin of the subject one or more peptides.

### CROSS-REFERENCE TO RELATED APPLICATIONS

This application claims priority and benefit from U.S. Provisional Application No. 63/053,484, filed Jul. 17, 2020, U.S. Provisional Application No. 63/143,021, filed Jan. 28, 2021, and U.S. Provisional Application No. 63/147,235, filed Feb. 8, 2021, the contents of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Jul. 16, 2021, is named 104545-0061-101-SL.txt and is 149,920 bytes in size.

### BACKGROUND OF THE DISCLOSURE

The novel coronavirus is referred to as SARS-CoV-2 or 2019-nCoV and is related to Severe Acute Respiratory Syndrome coronavirus (SARS-CoV), although with only approximately 80% similarity at the nucleotide level. Ralph et al. J Infect Dev Ctries. 2020 Jan. 31; 14(1):3-17. The infectious disease caused by SARS-CoV-2 has been named COVID-19 and its symptoms, depending on the infective strain, include fever, cough, fatigue, shortness of breath, and loss of smell and taste.

Coronaviruses are enveloped single stranded RNA viruses with positive-sense RNA genomes ranging from 25.5 to ˜32 kb in length. The spherical virus particles range from 70-120 nm in diameter and contain four structural proteins: the E and M proteins, which form the viral envelope; the N protein, which binds to the virus's RNA genome; and the S protein, which binds to human receptors. The genome of SARS-CoV-2 also comprises a number of open reading frames that code for a total of nine accessory proteins, which are not essential for virus replication.

Despite the fact that much effort is currently being invested into methods of detecting SARS-CoV-2, there is still a need to provide additional and improved approaches to detect SARS-CoV-2 and developed immunity, particularly cell-mediated immunity, to SARS-CoV-2. At present, the available methods to assess cell-mediated immune responses require highly specialized laboratories and in vitro techniques to detect cytokine synthesis or production following antigenic challenge in culture. Because these methods rely on surrogate markers, they may not reflect functional in vivo immunity. In addition, they are subject to inter-lab variability, expensive, time consuming and challenging to scale up. Thus, there is a need for improved methods for assessing cell-mediated immune responses to SARS-CoV-2 infection and/or vaccination.

### SUMMARY OF THE DISCLOSURE

In some aspects, the present disclosure provides methods and kits for detecting cell-mediated SARS-CoV-2 immunity in a subject, methods and kits for detecting a cell-mediated immune response to SARS-CoV-2 in a subject, methods and kits for detecting a SARS-CoV-2 infection in a subject and methods and kits for determining if a potential or approved vaccine against SARS-CoV-2 elicits a cell-mediated immune response in a subject.

The skin detection methods and kits of the disclosure are based on one or more peptides derived from one of the SARS-CoV-2 proteins and administering them to the skin of a subject. If the subject is infected with SARS-CoV-2, has had exposure to SARS-CoV-2 or has developed cell-mediated immunity to SARS-CoV-2 (e.g., by vaccination), these peptides elicit a cellular immunity response or cell-mediated immune response (i.e., CD4⁺ and/or CD8⁺ T cell responses) that can be detected by inspection of the area of the skin where the peptide(s) has been administered. One of the benefits of using the peptides disclosed herein, rather than larger proteins, is that smaller peptides are less likely to trigger IgE immune reactions in sensitized hosts. Another advantage of using the peptides disclosed herein is that the peptides are less likely to elicit a humoral response and the individual, therefore, does not produce antibodies against the peptides, as might be the case using the whole protein or a protein derivative. A further benefit of the methods of the disclosure is that they can detect SARS-CoV-2 immunity in a subject, even if the subject is seronegative and as well as in subjects with a history of asymptomatic or mild COVID-19. Additional benefits include ease of administration and timely read out, scalability and cost.

In a first aspect, the disclosure relates to a method for detecting SARS-CoV-2 cell-mediated immunity in a subject, the method comprising administering to the skin of the subject one or more peptides, the one or more peptides being selected from the group consisting of 10-25-mers than span sequentially and/or in overlapping format the SPIKE protein of the SARS-CoV-2 virus or a variant thereof. In other embodiments of this disclosure, the peptides of the group span at least 90%, at least 80%, at least 70%, at least 60% or at least 50% of the SPIKE protein of the SARs-CoV-2 virus or a variant thereof. In some aspects of this disclosure, the one or more peptides is selected from the group consisting peptides characterized by the amino acid sequences of SEQ ID NO: 1-104, 109-138, 140-167, 235-283, 294, 299, 303, 307-312, 317, 319, 320, 324-327, 333 and 335-337. In some aspects of this disclosure, the one or more peptides is selected from the group consisting peptides characterized by the amino acid sequences of SEQ ID NO: 1-104, 109-138, 140-183, 189-2312, 235-289, 294, 299, 303, 307-312, 317, 319, 320, 324-327, 333, 335-337, 341, 342 and 345-350. In some aspects of this disclosure, the one or more peptides is selected from the group consisting peptides being characterized by the amino acid sequences of SEQ ID NO: 235, 240, 241, 247-251, 253, 255-259, 264, 266, 268, 275, and 276. In some aspects of this disclosure, the one or more peptides is selected from the group consisting peptides being characterized by the amino acid sequences of SEQ ID NO: 236, 237, 242, 245, 246, 254-259, 264, 266, 267, 272, 274, and 280. In some aspects of this disclosure, the one or more peptides is selected from the group consisting peptides being characterized by the amino acid sequences of SEQ ID NO: 238, 239, 243, 244, 257-261, 264, 266, 268, 277-279, 281 and 283. In some aspects of this disclosure, the one or more peptides is selected from the group consisting peptides characterized by the amino acid sequences of SEQ ID NO: 1-358. In some aspects of this disclosure, the one or more peptides is selected from the group consisting peptides characterized by the amino acid sequences of SEQ ID NO: 1-183, 189-231 and 234-358. In some aspects of this disclosure, the one or more peptides is selected from the group consisting peptides characterized by the amino acid sequences of SEQ ID NO: 1-14, 16-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-168, 170, 171, 173, 175, 176, 178, 179, 181, 190, 235-248, 250-255, 257-261, 266, 268, 265, 272, 274, 275, 277-283, 290-306, 308-322, 324, 325, 328, 328, 330-334, 337-340, and 342-358.

In some aspects of this disclosure, the group of 10-25-mers does not include one or both of the peptides from circulating non-pandemic coronaviruses, e.g., cold viruses, and/or peptides from or spanning the superantigen domain of the SPIKE protein of the SARS-CoV-2 virus or variants thereof (see Cheng et al. 2020, PNAS 117:25254 (incorporated by reference herein))

In some aspects of this disclosure, the one or more peptides selected from the group consisting of the 10-25-mers are selected from combinations consisting of at least 25, at least 50, at least 75, at least 100, at lease 125, at least 150, at least 175 or at least 200 of the peptides of that group or 10-25-mers.

In another aspect, the disclosure relates to a method for detecting SARS-CoV-2 cell-mediated immunity in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by the amino acid sequences set forth in SEQ ID NO: 1-104, 109-138, 140-183, 189-231, 235-289, 294, 299, 303, 307-312, 317, 319, 320, 324-327, 333, 335-337, 341, 342 and 345-350 and inspecting the skin region in the area of the administration for an immune reaction, wherein the presence of an immune reaction is indicative of the subject having developed cell-mediated immunity to SARS-CoV-2.

In a another aspect, the disclosure relates to a method for detecting a cell-mediated immune response to SARS-CoV-2 in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by the amino acid sequences set forth in SEQ ID NO: 1-104, 109-138, 140-183, 189-231, 235-289, 294, 299, 303, 307-312, 317, 319, 320, 324-327, 333, 335-337, 341, 342 and 345-350 and inspecting the skin region in the area of the administration for an immune reaction, wherein the presence of an immune reaction is indicative of the subject having developed a cell-mediated immune response to SARS-CoV-2.

In a another aspect, the disclosure relates to a method for determining if a potential or approved vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by the amino acid sequences set forth in SEQ ID NO: 1-104, 109-138, 140-183, 189-231, 235-289, 294, 299, 303, 307-312, 317, 319, 320, 324-327, 333, 335-337, 341, 342 and 345-350 and inspecting the skin region in the area of the administration for an immune reaction, wherein the presence of an immune reaction is indicative of the vaccine eliciting a cell-mediated immune response to SARS-CoV-2 in the subject.

In a another aspect, the disclosure relates to a method for determining if a potential or approved vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by the amino acid sequences set forth in SEQ ID NO: 1-14, 16-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-168, 170, 171, 173, 175, 176, 178, 179, 181, 190, 235-248, 250-255, 257-261, 266, 268, 265, 272, 274, 275, 277-283, 290-306, 308-322, 324, 325, 328, 328, 330-334, 337-340, and 342-358 and inspecting the skin region in the area of the administration for an immune reaction, wherein the presence of an immune reaction is indicative of the vaccine eliciting a cell-mediated immune response to SARS-CoV-2 in the subject.

In some embodiments, the methods and kits of this disclosure are characterized by a delayed type hypersensitivity (DTH) skin test to demonstrate the presence of functional cell-mediated immune responses (CMI) to SARS-CoV-2.

In some embodiments, the methods and kits of this disclosure are useful as one or more of: 1) a diagnostic tool for SARS-CoV-2 exposure and T-cell immunity, 2) a correlate of protective immunity, 3) a method to stratify participants in vaccine trials, 4) a surrogate marker in vaccine trials to evaluate CMI responses to vaccines, and 5) a method to measure the durability of CMI in the weeks/months/years following natural infection or vaccination.

In some embodiments, the administration of the one or more peptides is selected from the group consisting of cutaneous, intradermal, transdermal and subcutaneous administration.

In some embodiments of the methods and kits of the disclosure, the one, five, ten, fifteen, twenty, twenty-five, thirty or more peptides are selected from the group consisting of peptides being characterized by the amino acid sequences set forth in SEQ ID NO: 1-45. Optionally, the one or more peptides are selected from the group consisting of peptides being characterized by the amino acid sequences set forth in SEQ ID NO: 1-23. Optionally, the one or more peptides are selected from the group consisting of peptides being characterized by the amino acid sequences set forth in SEQ ID NO: 26-28. Optionally, the one or more peptides are selected from the group consisting of peptides being characterized by the amino acid sequences set forth in SEQ ID NO: 29-38. Optionally, the one or more peptides are selected from the group consisting of peptides the amino acid sequences set forth in SEQ ID NO: 24-25 and SEQ ID NO: 39-45. Optionally, the one or more peptides are selected from the group consisting of peptides being characterized by the amino acid sequences set forth in SEQ ID NO: 24-25 and SEQ ID NO: 39-45 and 194-231.

In some embodiments of the methods, kits, and combinations or pools of the disclosure, the one, five, ten, fifteen, twenty or more peptides are selected from the group consisting of peptides being characterized by the amino acid sequences set forth in SEQ ID NO: 1-10, 12-25, 27, 28, 30-39, 41, 43 and 44. Optionally, the one, five, ten, fifteen or more peptides are selected from the group consisting of peptides being characterized by the amino acid sequences set forth in SEQ ID NO: 1-10 and 12-23. Optionally, the one or more peptides are selected from the group consisting of peptides being characterized by the amino acid sequences set forth in SEQ ID NO: 27 and 28. Optionally, the one or more peptides are selected from the group consisting of peptides being characterized by the amino acid sequences set forth in SEQ ID NO: 30-38. Optionally, the one or more peptides are selected from the group consisting of peptides being characterized by the amino acid sequences set forth in SEQ ID NO: 24-25 and SEQ ID NO: 29, 41, 43 and 44.

In some embodiments of the methods, kits, and combinations or pools of the disclosure, the one, five, ten, fifteen, twenty, twenty-five, thirty or more peptides are selected from the group consisting of peptides being characterized by the amino acid sequences set forth in SEQ ID NO: 1-14, 16-45 and 292. Optionally, the one, five, ten, fifteen or more peptides are selected from the group consisting of peptides being characterized by the amino acid sequences set forth in SEQ ID NO: 11, 16, 19, 26, 29, 40, 42, and 45. Optionally, the one or more peptides are selected from the group consisting of peptides being characterized by the amino acid sequences set forth in SEQ ID NO: 1-10, 12-14, 17, 18, 20-25, 27, 28, 30-39, 41, 43, 44 and 292.

In some embodiments of the methods, kits, and combinations or pools of the disclosure, the one, five, ten, fifteen, twenty, twenty-five, thirty or more peptides are selected from the group consisting of peptides being characterized by the amino acid sequences set forth in SEQ ID NO: 46-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-167, 265, 294, 299, 300, 303, 308, 309, 311, 312, 317-321, 324, 325, 330-334, 337-340, and 354-357. Optionally, the one, five, ten, fifteen or more peptides are selected from the group consisting of peptides being characterized by the amino acid sequences set forth in SEQ ID NO: 47, 48, 50-55, 59, 60, 62-64, 66-70, 72, 74, 75, 77, 79-81, 83, 84, 86, 87, 90-94, 96, 99-101, 103, 110, 115-125, 129, 131-133, 136-138, 140, 142, 144, 146, 148,149, 153-155, 158, 159, 294, 299, 300, 303, 308, 309, 311, 312, 317-321, 324, 325, 330-334, 337-340, and 354-357. Optionally, the one or more peptides are selected from the group consisting of peptides being characterized by the amino acid sequences set forth in SEQ ID NO: 46, 47, 58, 61, 71, 76, 89, 105-109, 111, 113, 114, 126, 128, 130, 139, 141, 147, 151, 152, 160-167, and 265.

In some embodiments of the methods, kits, and combinations or pools of the disclosure, the one, five, ten, fifteen or more peptides are selected from the group consisting of peptides being characterized by the amino acid sequences set forth in SEQ ID NO: 168, 170, 171, 173, 175, 176, 178, 179, 181, 190, and 342-350. Optionally, the one, five, ten, fifteen or more peptides are selected from the group consisting of peptides being characterized by the amino acid sequences set forth in SEQ ID NO: 170, 175, 176, 178, 179, 181, 190, and 342-350. Optionally, the one or more peptides are selected from the group consisting of peptides being characterized by the amino acid sequences set forth in SEQ ID NO: 168, 171, and 173.

In some embodiments of the methods, kits, and combinations or pools of the disclosure, the one, five, ten, fifteen, twenty, twenty-five, thirty or more peptides are selected from the group consisting of peptides being characterized by the amino acid sequences set forth in SEQ ID NO: 235-248, 250-255, 257-261, 266, 268, 272, 274, 275, 277-283, 290, 291, 351-353 and 358. Optionally, the one, five, ten, fifteen or more peptides are selected from the group consisting of peptides being characterized by the amino acid sequences set forth in SEQ ID NO: 235-248, 250, 252-255, 257-261, 268, 278-281, 290, 291, 351-353 and 358. Optionally, the one or more peptides are selected from the group consisting of peptides being characterized by the amino acid sequences set forth in SEQ ID NO: 251, 266, 272, 274, 275, 277, 282 and 283.

In some embodiments of the methods, kits, and combinations or pools of the disclosure, the one, five, ten, fifteen or more peptides are selected from the group consisting of peptides being characterized by the amino acid sequences set forth in SEQ ID NO: 293, 295-298, 301, 302, 304-306, 313-316, 322, 328 and 329.

In some embodiments of the methods of the disclosure, the one or more peptides are selected from the group consisting of peptides being characterized by the amino acid sequences set forth in SEQ ID NO: 46-167.

In some embodiments of the methods of the disclosure, the one or more peptides are selected from the group consisting of peptides being characterized by the amino acid sequences set forth in SEQ ID NO: 46-70, 72-94, 96, 97, 99-101, 103-107, 109-111, 113-149, 151-167, 307, 317, 319, 320, 323, 324 and 335.

In some embodiments of the methods, kits and pools or combinations of the disclosure, the one or more peptides are selected from the group consisting of peptides being characterized by the amino acid sequences set forth in SEQ ID NO: 47, 50-55, 59, 60, 62-64, 66-70, 72, 74-77, 79-81, 83, 84, 86, 87, 90-94, 96, 97, 99-101, 103, 110, 114-126, 129, 131-134, 135-138, 140, 144-146, 148,149, 153-167, 294, 299, 300, 303, 307, 308, 310, 311, 312, 317, 319, 320, 324, 325, 327, 333, 336 and 337, and optionally one or more peptides being characterized by the amino acid sequences set forth in SEQ ID NO: 235-282.

In some embodiments of the methods of the disclosure, the one or more peptides are selected from the group consisting of peptides being characterized by the amino acid sequences set forth in SEQ ID NO: 47, 50-55, 59, 60, 62-64, 66-70, 72, 74-77, 79-81, 83, 84, 86, 87, 90-94, 96, 97, 99-101, 103, 110, 114-126, 129, 131-134, 135-138, 140, 144-146, 148,149, 153-167, 235-282, 294, 299, 300, 303, 307, 308, 310, 311, 312, 317, 319, 320, 324, 325, 327, 333, 336 and 337.

In some embodiments of the methods of the disclosure, the one or more peptides are selected from the group consisting of peptides being characterized by the amino acid sequences set forth in SEQ ID NO: 168-183. In some embodiments of the methods of the disclosure, the one or more peptides are selected from the group consisting of peptides being characterized by the amino acid sequences set forth in SEQ ID NO: 168-183 and 189-193. Optionally, the one or more peptides are selected from the group consisting of peptides being characterized by the amino acid sequences set forth in SEQ ID NO: 168-172. Optionally, the one or more peptides are selected from the group consisting of peptides being characterized by the amino acid sequences set forth in SEQ ID NO: 173-183. Optionally, the one or more peptides are selected from the group consisting of peptides being characterized by the amino acid sequences set forth in SEQ ID NO: 173-183, 189 and 190.

In some embodiments of the methods of the disclosure, the one or more peptides are selected from the group consisting of peptides being characterized by the amino acid sequences set forth in SEQ ID NO: 168, 170-183, 189-193 and 342. Optionally, the one or more peptides are selected from the group consisting of peptides being characterized by the amino acid sequences set forth in SEQ ID NO: 168, 170-172 and 342. Optionally, the one or more peptides are selected from the group consisting of peptides being characterized by the amino acid sequences set forth in SEQ ID NO: 173-183. Optionally, the one or more peptides are selected from the group consisting of peptides being characterized by the amino acid sequences set forth in SEQ ID NO: 173-183, 189 and 190.

In some embodiments of the methods of the disclosure, the one or more peptides are selected from the group consisting of peptides being characterized by the amino acid sequences set forth in SEQ ID NO: 170-183, 189-193, 341 and 342. Optionally, the one or more peptides are selected from the group consisting of peptides being characterized by the amino acid sequences set forth in SEQ ID NO: 170-172, 341 and 342. Optionally, the one or more peptides are selected from the group consisting of peptides being characterized by the amino acid sequences set forth in SEQ ID NO: 173-183. Optionally, the one or more peptides are selected from the group consisting of peptides being characterized by the amino acid sequences set forth in SEQ ID NO: 173-183, 189 and 190.

In some embodiments of the methods, kits and peptide combinations or pools of the disclosure, the one or more peptides are selected from the group consisting of peptides being characterized by the amino acid sequences set forth in SEQ ID NO: 170, 172, 174-175, 178, 179, 181, 190, 341, 342 and 345-350, and optionally one or more peptides being characterized by the amino acid sequences set forth in SEQ ID NO: 284-289. In some embodiments of the methods of the disclosure, the one or more peptides are selected from the group consisting of peptides being characterized by the amino acid sequences set forth in SEQ ID NO: 170, 172, 174-175, 178, 179, 181, 190, 284-289, 341, 342 and 345-350. Optionally, the one or more peptides are selected from the group consisting of peptides being characterized by the amino acid sequences set forth in SEQ ID NO: 170, 172, 341 and 342. Optionally, the one or more peptides are selected from the group consisting of peptides being characterized by the amino acid sequences set forth in SEQ ID NO: 174-176, 178, 179, 181, 190 and 345-350. Optionally, the one or more peptides are selected from the group consisting of peptides being characterized by the amino acid sequences set forth in SEQ ID NO: 174-176, 178, 179, 181, 190, 284-289 and 345-350.

In some embodiments of the methods of the disclosure, the one or more peptides are selected from the group consisting of peptides being characterized by the amino acid sequences set forth in SEQ ID NO: 191-193.

In some embodiments of the methods of the disclosure, the one or more peptides is a combination of all the peptides being characterized by the amino acid sequences set forth in SEQ ID NO: 1-45. In some embodiments, the one or more peptides is a combination of all the peptides being characterized by the amino acid sequences set forth in SEQ ID NO: 46-167. In some embodiments, the one or more peptides is a combination of all the peptides being characterized by the amino acid sequences set forth in SEQ ID NO: 168-183. In some embodiments, the one or more peptides is a combination of all the peptides being characterized by the amino acid sequences set forth in SEQ ID NO: 168-183, 189 and 190.

In some embodiments of the methods of the disclosure, the one or more peptides is at least a combination of peptides being characterized by the amino acid sequences set forth in SEQ ID NO: 1-45. In some embodiments, the one or more peptides is at least a combination of peptides being characterized by the amino acid sequences set forth in SEQ ID NO: 46-167. In some embodiments, the one or more peptides is at least a combination of peptides being characterized by the amino acid sequences set forth in SEQ ID NO: 168-183. In some embodiments, the one or more peptides is at least a combination of peptides being characterized by the amino acid sequences set forth in SEQ ID NO: 168-183, 189 and 190.

In some embodiments, the one or more peptides is at least a combination of peptides being characterized by the amino acid sequences set forth in SEQ ID NO: 46-70, 72-94, 96, 97, 99-101, 103-107, 109-111, 113-149, 151-167, 307, 317, 319, 320, 323, 324 and 335. In some embodiments, the one or more peptides is at least a combination of peptides being characterized by the amino acid sequences set forth in SEQ ID NO: 170-183, 189, 190, 341 and 342. In some embodiments, the one or more peptides is at least a combination of peptides being characterized by the amino acid sequences set forth in SEQ ID NO: 170-183, 189, 190, 341 and 342.

In some embodiments of the methods of the disclosure, the one or more peptides is at least a combination of peptides being characterized by the amino acid sequences set forth in SEQ ID NO: 1-10, 12-25, 27, 28, 30-39, 41, 43 and 44. In some embodiments of the methods of the disclosure, the one or more peptides is at least a combination of peptides being characterized by the amino acid sequences set forth in SEQ ID NO: 47, 50-55, 59, 60, 62-64, 66-70, 72, 74-77, 79-81, 83, 84, 86, 87, 90-94, 96, 97, 99-101, 103, 110, 114-126, 129, 131-134, 135-138, 140, 144-146, 148,149, 153-167, 294, 299, 300, 303, 307, 308, 310-312, 317, 319, 320, 324, 325, 327, 333, 336 and 337, and optionally one or more peptides being characterized by the amino acid sequences set forth in SEQ ID NO: 235-282. In some embodiments of the methods of the disclosure, the one or more peptides is at least a combination of peptides being characterized by the amino acid sequences set forth in SEQ ID NO: 47, 50-55, 59, 60, 62-64, 66-70, 72, 74-77, 79-81, 83, 84, 86, 87, 90-94, 96, 97, 99-101, 103, 110, 114-126, 129, 131-134, 135-138, 140, 144-146, 148,149, 153-167, 235-282, 294, 299, 300, 303, 307, 308, 310-312, 317, 319, 320, 324, 325, 327, 333, 336 and 337. In some embodiments of the methods of the disclosure, the one or more peptides is at least a combination of peptides being characterized by the amino acid sequences set forth in 170, 172, 341 and 342. In some embodiments of the methods of the disclosure, the one or more peptides is at least a combination of peptides being characterized by the amino acid sequences set forth in SEQ ID NO: 174-176, 178, 179, 181, 190 and 345-350, and optionally one or more peptides being characterized by the amino acid sequences set forth in SEQ ID NO: 284-289. In some embodiments of the methods of the disclosure, the one or more peptides is at least a combination of peptides being characterized by the amino acid sequences set forth in SEQ ID NO: 174-176, 178, 179, 181, 190, 284-289 and 345-350.

In some embodiments, the one or more peptides excludes a superantigen region. In some embodiments, the one or more peptides excludes a peptide sequence similar to or the same as another coronavirus spike protein, e.g., peptides related to cold viruses.

In some embodiments, the inspecting step is a visual inspection. In some embodiments, the inspection of the skin region takes place within 24-72 hours after administration of the one or more peptides.

In some embodiments, at least one or more of the peptides are in a lyophilized form. In some embodiments, at least one or more of the peptides are dissolved in a solvent. Optionally, if one or more of the peptides is present in the form of a lyophilizate, the one or more peptides are reconstituted in a solvent before the administration to the skin. Optionally, the solvent is selected from the group consisting of glycerol, water for injection, a phosphate buffered saline, a sodium phosphate buffer, a Tris buffer, a borate buffer, a succinate buffer, a histidine buffer, a citrate buffer, a potassium phosphate buffer and a mannitol solution. In some embodiments, the solvent further comprises at least one preservative. Optionally, the preservative is selected from the group consisting of phenol, m-cresol, thiomersal, 2-phenoxyethanol and 8-hydroxy quinoline. In some embodiments, the solvent further comprises at least one excipient. Optionally, the excipient is selected from the group consisting of mannitol, trehalose, sucrose and sorbitol. In some embodiments, the solvent further comprises at least one detergent. Optionally, the detergent is selected from the group consisting of Polysorbate 20, Polysorbate 80, Poloxamer 188 and other Poloxamers, Triton X-100, polyoxyethylene sorbitan, fatty acid esters, polyoxyl-40-stearate and other polyoxyethylene stearates, glycerol monostearate, macrogol-8-stearat, macrogol cetostearylether 20 and polyoxyethylene alkyl ethers, sorbitan monostearate and other sorbitan monoesters, polyoxyethylene castor oil derivatives, sodium lauryl sulfate and cetylpyridinium chloride. Optionally, if one or more of the peptides is present in the form of a lyophilizate, the one or more peptides in the lyophilizate are reconstituted in a solvent and combined with other one or more peptides, either reconstituted from a lyophilizate or in initial soluble form and then the combined reconstituted peptides are lyophilized, and then the lyophilized group is reconstituted before administration to the skin (see e.g., Carrasco Pro et al., “Automatic Generation of Validated Specific Epitope Sets,” Journal of Immunology Research, 2015: 763461 (2015), incorporated herein by reference).

In some embodiments, the amount of the each of the one or more peptides is 0.01 to 50 μg.

In some embodiments, the immune reaction is a reaction in or on the skin. Optionally, the immune reaction comprises an induration having a mean diameter of more than about 3 mm in or on the skin. Optionally, the immune reaction comprises an induration having a mean diameter of more than about 5 mm in or on the skin.

In some embodiments, the one or more peptides are administered to the skin with a syringe, a microneedle patch or a lancet.

In a another aspect, the disclosure relates to a kit for detecting a cell mediated immune response to SARS-CoV-2 in a subject, said kit comprising one or more peptides, the peptides being selected from the group consisting of peptides being characterized by the amino acid sequences set forth in SEQ ID NO: 1-104, 109-138, 140-183, 189-231, 235-289, 294, 299, 303, 307-312, 317, 319, 320, 324-327, 333, 335-337, 341, 342 and 345-350 and instructions for its use.

In some embodiments of the kit of the disclosure, the one or more peptides are selected from the group consisting of peptides being characterized by the amino acid sequences set forth in SEQ ID NO: 1-45. Optionally, the one or more peptides are selected from the group consisting of peptides being characterized by the amino acid sequences set forth in SEQ ID NO: 1-23. Optionally, the one or more peptides are selected from the group consisting of peptides being characterized by the amino acid sequences set forth in SEQ ID NO: 26-28. Optionally, the one or more peptides are selected from the group consisting of peptides being characterized by the amino acid sequences set forth in SEQ ID NO: 29-38. Optionally, the one or more peptides are selected from the group consisting of peptides being characterized by the amino acid sequences set forth in SEQ ID NO: 24-25 and SEQ ID NO: 39-45. Optionally, the one or more peptides are selected from the group consisting of peptides being characterized by the amino acid sequences set forth in SEQ ID NO: 24-25 and SEQ ID NO: 39-45 and 194-231.

In some embodiments of the kit of the disclosure, the one, five, ten, fifteen, twenty, twenty-five, thirty or more peptides are selected from the group consisting of peptides being characterized by the amino acid sequences set forth in SEQ ID NO: 1-10, 12-25, 27, 28, 30-39, 41, 43 and 44. Optionally, the one or more peptides are selected from the group consisting of peptides being characterized by the amino acid sequences set forth in SEQ ID NO: 1-10 and 12-23. Optionally, the one or more peptides are selected from the group consisting of peptides being characterized by the amino acid sequences set forth in SEQ ID NO: 27 and 28. Optionally, the one or more peptides are selected from the group consisting of peptides being characterized by the amino acid sequences set forth in SEQ ID NO: 30-38. Optionally, the one or more peptides are selected from the group consisting of peptides being characterized by the amino acid sequences set forth in SEQ ID NO: 24-25 and SEQ ID NO: 29, 41, 43 and 44.

In some embodiments of the kit of the disclosure, the one or more peptides are selected from the group consisting of peptides being characterized by the amino acid sequences set forth in SEQ ID NO: 46-167.

In some embodiments of the kit of the disclosure, the one or more peptides are selected from the group consisting of peptides being characterized by the amino acid sequences set forth in SEQ ID NO: 46-70, 72-94, 96, 97, 99-101, 103-107, 109-111, 113-149, 151-167, 307, 317, 319, 320, 323, 324 and 335.

In some embodiments of the kit of the disclosure, the one or more peptides are selected from the group consisting of peptides being characterized by the amino acid sequences set forth in SEQ ID NO: 47, 50-55, 59, 60, 62-64, 66-70, 72, 74-77, 79-81, 83, 84, 86, 87, 90-94, 9596, 97, 99-101, 103, 110, 114-126, 129, 131-134, 135-138, 140, 144-146, 148,149, 153-167, 294, 299, 300, 303, 307, 308, 310, 311, 312, 317, 319, 320, 324, 325, 327, 333, 336 and 337, and optionally one or more peptides being characterized by the amino acid sequences set forth in SEQ ID NO: 235-282.

In some embodiments of the kit of the disclosure, the one or more peptides are selected from the group consisting of peptides being characterized by the amino acid sequences set forth in SEQ ID NO: 47, 294, 50-55, 299, 300, 59, 60, 62-64, 66-70, 72, 74-77, 79-81, 83, 8486, 87, 90-94, 96, 97, 99-101, 103, 110, 114-126, 129, 131-134, 135-138, 140, 144-146, 148,149, 153-167, 235-282, 294, 299, 300, 303, 307, 308, 310, 311, 312, 317, 319, 320, 324, 325, 327, 333, 336 and 337.

In some embodiments of the kit of the disclosure, the one or more peptides are selected from the group consisting of peptides being characterized by the amino acid sequences set forth in SEQ ID NO: 168-183. Optionally, the one or more peptides are selected from the group consisting of peptides being characterized by the amino acid sequences set forth in SEQ ID NO: 168-172. Optionally, the one or more peptides are selected from the group consisting of peptides being characterized by the amino acid sequences set forth in SEQ ID NO: 173-183.

In some embodiments of the kit of the disclosure, the one or more peptides are selected from the group consisting of peptides being characterized by the amino acid sequences set forth in SEQ ID NO: 168-183, 189 and 190. Optionally, the one or more peptides are selected from the group consisting of peptides being characterized by the amino acid sequences set forth in SEQ ID NO: 168-172. Optionally, the one or more peptides are selected from the group consisting of peptides being characterized by the amino acid sequences set forth in SEQ ID NO: 173-183, 189 and 190.

In some embodiments of the kit of the disclosure, the one or more peptides are selected from the group consisting of peptides being characterized by the amino acid sequences set forth in SEQ ID NO: 168, 170-183, 189, 190, 342. Optionally, the one or more peptides are selected from the group consisting of peptides being characterized by the amino acid sequences set forth in SEQ ID NO: 168, 170-172 and 342. Optionally, the one or more peptides are selected from the group consisting of peptides being characterized by the amino acid sequences set forth in SEQ ID NO: 173-183, 189 and 190.

In some embodiments of the kit of the disclosure, the one or more peptides are selected from the group consisting of peptides being characterized by the amino acid sequences set forth in SEQ ID NO: 170-183, 189, 190, 341 and 342. Optionally, the one or more peptides are selected from the group consisting of peptides being characterized by the amino acid sequences set forth in SEQ ID NO: 170-172, 341 and 342. Optionally, the one or more peptides are selected from the group consisting of peptides being characterized by the amino acid sequences set forth in SEQ ID NO: 173-183, 189 and 190.

In some embodiments of the kit of the disclosure, the one or more peptides are selected from the group consisting of peptides being characterized by the amino acid sequences set forth in SEQ ID NO: 170, 172, 174-175, 178, 179, 181, 190, 341, 342 and 345-350, and optionally one or more peptides being characterized by the amino acid sequences set forth in SEQ ID NO: 284-289. In some embodiments of the kit of the disclosure, the one or more peptides are selected from the group consisting of peptides being characterized by the amino acid sequences set forth in SEQ ID NO: 170, 172, 174-175, 178, 179, 181, 190, 284-289, 341, 342 and 345-350. Optionally, the one or more peptides are selected from the group consisting of peptides being characterized by the amino acid sequences set forth in SEQ ID NO: 170, 172, 341 and 342. Optionally, the one or more peptides are selected from the group consisting of peptides being characterized by the amino acid sequences set forth in SEQ ID NO: 174-175, 178, 179, 181, 190 and 345-350. Optionally, the one or more peptides are selected from the group consisting of peptides being characterized by the amino acid sequences set forth in SEQ ID NO: 174-175, 178, 179, 181, 190, 284-289 and 345-350.

In some embodiments of the kit of the disclosure, the one or more peptides are selected from the group consisting of peptides being characterized by the amino acid sequences set forth in SEQ ID NO: 191-193.

In some embodiments of the kit of the disclosure, the one, five, ten, fifteen, twenty, twenty-five, thirty or more peptides are selected from the group consisting of peptides being characterized by the amino acid sequences set forth in SEQ ID NO: 1-10, 12-14, 17, 18, 20-25, 27, 28, 30-39, 41, 43, 44 and 292, and optionally one or more sequences characterized by the amino acid sequences set forth in SEQ ID NO: 11, 16, 19, 26, 29, 40, 42, and 45.

In some embodiments of the kit of the disclosure, the one, five, ten, fifteen, twenty, twenty-five, thirty or more peptides are selected from the group consisting of peptides being characterized by the amino acid sequences set forth in SEQ ID NO: 47, 48, 50-55, 59, 60, 62-64, 66-70, 72, 74, 75, 77, 79-81, 83, 84, 86, 87, 90-94, 96, 99-101, 103, 110, 115-125, 129, 131-133, 136-138, 140, 142, 144, 146, 148,149, 153-155, 158, 159, 294, 299, 300, 303, 308, 309, 311, 312, 317-321, 324, 325, 330-334, 337-340, and 354-357, and optionally one or more peptides being characterized by the amino acid sequences set forth in SEQ ID NO: 46, 47, 58, 61, 71, 76, 89, 105-109, 111, 113, 114, 126, 128, 130, 139, 141, 147, 151, 152, 160-167, and 265.

In some embodiments of the kit of the disclosure, the one, five, ten or more peptides are selected from the group consisting of peptides being characterized by the amino acid sequences set forth in SEQ ID NO: 170, 175, 176, 178, 179, 181, 190, and 342-350, and optionally one or more peptides being characterized by the amino acid sequences set forth in SEQ ID NO: 168, 171 and 173.

In some embodiments of the kit of the disclosure, the one, five, ten, fifteen, twenty, twenty-five, thirty or more peptides are selected from the group consisting of peptides being characterized by the amino acid sequences set forth in SEQ ID NO: 235-248, 250, 252-255, 257-261, 268, 278-281, 290, 291, 351-353 and 358, and optionally one or more peptides comprising the amino acid sequences set forth in SEQ ID NO: 251, 266, 272, 274, 275, 277, 282 and 283.

In some embodiments of the kit of the disclosure, the one, five, ten, fifteen or more peptides are selected from the group consisting of peptides being characterized by the amino acid sequences set forth in SEQ ID NO: 293, 295-298, 301, 302, 304-306, 313-316, 322, 328, 329.

In some embodiments of the kit of the disclosure, the one or more peptides is a combination of all the peptides being characterized by the amino acid sequences set forth in SEQ ID NO: 1-45. In some embodiments, the one or more peptides is a combination of all the peptides being characterized by the amino acid sequences set forth in SEQ ID NO: 46-167. In some embodiments, the one or more peptides is a combination of all the peptides being characterized by the amino acid sequences set forth in SEQ ID NO: 168-183. In some embodiments, the one or more peptides is a combination of all the peptides being characterized by the amino acid sequences set forth in SEQ ID NO: 168-183, 189 and 190.

In some embodiments of the kit of the disclosure, the one or more peptides is at least a combination of peptides being characterized by the amino acid sequences set forth in SEQ ID NO: 1-45. In some embodiments, the one or more peptides is at least a combination of peptides being characterized by the amino acid sequences set forth in SEQ ID NO: 46-167. In some embodiments, the one or more peptides is at least a combination of peptides being characterized by the amino acid sequences set forth in SEQ ID NO: 168-183. In some embodiments, the one or more peptides is at least a combination of peptides being characterized by the amino acid sequences set forth in SEQ ID NO: 168-183, 189 and 190.

In some embodiments, the one or more peptides is at least a combination of peptides being characterized by the amino acid sequences set forth in SEQ ID NO: 46-70, 72-94, 96, 97, 99-101, 103-107, 109-111, 113-149, 151-167, 307, 317, 319, 320, 323, 324 and 335. In some embodiments, the one or more peptides is at least a combination of peptides being characterized by the amino acid sequences set forth in SEQ ID NO: 168, 170-183, 189, 190 and 342. In some embodiments, the one or more peptides is at least a combination of peptides being characterized by the amino acid sequences set forth in SEQ ID NO: 170-183, 189, 190, 341 and 342.

In some embodiments of the kit of the disclosure, the one or more peptides is at least a combination of peptides being characterized by the amino acid sequences set forth in SEQ ID NO: 1-10, 12-25, 27, 28, 30-39, 41, 43 and 44. In some embodiments, the one or more peptides is at least a combination of peptides being characterized by the amino acid sequences set forth in SEQ ID NO: 47, 50-55, 59, 60, 62-64, 66-70, 72, 74-77, 79-81, 83, 84, 86, 87, 90-94, 96, 97, 99-101, 103, 110, 114-126, 129, 131-134, 135-138, 140, 144-146, 148,149, 153-167, 294, 299, 300, 303, 307, 308, 310, 311, 312, 317, 319, 320, 324, 325, 327, 333, 336 and 337, and optionally one or more peptides being characterized by the amino acid sequences set forth in SEQ ID NO: 235-282. In some embodiments, the one or more peptides is at least a combination of peptides being characterized by the amino acid sequences set forth in SEQ ID NO: 47, 50-55, 59, 60, 62-64, 66-70, 72, 74-77, 79-81, 83, 84, 86, 87, 90-94, 97, 99-101, 103, 110, 114-126, 129, 131-134, 135-138, 140, 144-146, 148,149, 153-167, 235-282, 294, 299, 300, 303, 307, 308, 310, 311, 312, 317, 319, 320, 324, 325, 327, 333, 336 and 337. In some embodiments, the one or more peptides is at least a combination of peptides being characterized by the amino acid sequences set forth in SEQ ID NO: 170, 172, 174-175, 178, 179, 181, 190, 341, 342 and 345-350, and optionally one or more peptides being characterized by the amino acid sequences set forth in SEQ ID NO: 284-289. In some embodiments, the one or more peptides is at least a combination of peptides being characterized by the amino acid sequences set forth in SEQ ID NO: 170, 172, 174-175, 178, 179, 181, 190, 284-289, 341, 342 and 345-350.

In some embodiments of the kit of the disclosure, the one or more peptides comprise the peptides of one or more of Pool 1, Pool 2, and Pool 3. In some embodiments of the kit of the disclosure, the one or more peptides comprise the peptides of one or more of Pool 4, Pool 5, and Pool 6. In some embodiments of the kit of the disclosure, the one or more peptides comprise the peptides of one or more of Pool 7, Pool 8, and Pool 9. In some embodiments of the kit of the disclosure, the one or more peptides comprise the peptides of one or more of Pool 4, Pool 5, Pool 6 and Pool 10. In some embodiments of the kit of the disclosure, the one or more peptides comprise the peptides of one or more of Pool 4, Pool 5, Pool 6, Pool 10 and Pool 11. In some embodiments of the kit of the disclosure, the one or more peptides comprise the peptides of one or more of Pool 7, Pool 8, Pool 9 and Pool 10. In some embodiments of the kit of the disclosure, the one or more peptides comprise the peptides of one or more of Pool 7, Pool 8, Pool 9, Pool 10 and Pool 11.

In some embodiments of the kit of the disclosure, the kit further comprises an applicator to administer the one or more peptides. Optionally, the applicator is a syringe, a microneedle patch or a lancet.

In some embodiments of the kit of the disclosure, the one or more peptides are present in the form of a lyophilizate. In some embodiments, the kit further comprises a solvent to dissolve or to reconstitute the one or more lyophilized peptides. In some embodiments of the kit of the disclosure, at least one or more of the peptides are present in a solvent. Optionally, the solvent is selected from the group consisting of glycerol, water for injection, a phosphate buffered saline, a sodium phosphate buffer, a Tris buffer, a borate buffer, a succinate buffer, a histidine buffer, a citrate buffer, a potassium phosphate buffer and a mannitol solution. In some embodiments, the solvent further comprises at least one preservative. Optionally, the preservative is selected from the group consisting of phenol, m-cresol, thiomersal, 2-phenoxyethanol and 8-hydroxy quinoline. In some embodiments, the solvent further comprises at least one excipient. Optionally, the excipient is selected from the group consisting of mannitol, trehalose, sucrose and sorbitol. In some embodiments, the solvent further comprises at least one detergent. Optionally, the detergent is selected from the group consisting of Polysorbate 20, Polysorbate 80, Poloxamer 188 and other Poloxamers, Triton X-100, polyoxyethylene sorbitan, fatty acid esters, polyoxyl-40-stearate and other polyoxyethylene stearates, glycerol monostearate, macrogol-8-stearat, macrogol cetostearylether 20 and polyoxyethylene alkyl ethers, sorbitan monostearate and other sorbitan monoesters, polyoxyethylene castor oil derivatives, sodium lauryl sulfate and cetylpyridinium chloride.

In another aspect, the disclosure relates to a method to stratify subjects in a vaccine trial, the method comprising detecting a cell-mediated immune response in the various participants before, during and after the trial.

In another aspect, the disclosure relates to a method to determine a surrogate marker in vaccine trials, the method comprising detecting a cell-mediated immune response in the subject by using the kit, wherein if a cell-mediated immune response is observed, the subject is infected with SARS-CoV-2.

In another aspect, the disclosure relates to a method to measure the durability of a cell-mediate immune response in the weeks following natural infection or vaccination, the method comprising detecting a cell-mediated immune response in the subject by using the kit.

In another aspect, the disclosure relates to a method to measure the durability of a cell-mediate immune response in the months following natural infection or vaccination, the method comprising detecting a cell-mediated immune response in the subject by using the kit.

In another aspect, the disclosure relates to a method to measure the durability of a cell-mediate immune response in the years following natural infection or vaccination, the method comprising detecting a cell-mediated immune response in the subject by using the kit.

In some embodiments of the method of the disclosure, the peptides comprise the peptides of one or more of Pool 1, Pool 2, and Pool 3.

In some embodiments of the method of the disclosure, the peptides comprise a combination of all the peptides of Pool 1, Pool 2, and Pool 3.

In some embodiments of the kit of the disclosure comprises the peptides one or more of Pool 1, Pool 2, and Pool 3.

In some embodiments of the kit of the disclosure comprises a combination of all the peptides of Pool 1, Pool 2, and Pool 3.

In some embodiments of the combination or pool of peptides of the disclosure, the combination or pool of peptides being characterized by the amino acid sequences set forth in SEQ ID NO: 1-10, 12-25, 27, 28, 30-39, 41, 43 and 44. In some embodiments of the combination or pool of peptides of the disclosure, the combination or pool of peptides being characterized by the amino acid sequences set forth in SEQ ID NO: 47, 49a, 50-55, 56a, 56b, 59, 60, 62-64, 65a, 66-70, 71a, 72, 73a, 74-77, 79-81, 82a, 83, 84, 85a, 86, 87, 88a, 90-94, 95a, 96, 97, 98a, 99-101, 102a, 103, 110, 112a, 114-126, 127a, 129, 131-134, 134a, 135-138, 140, 143a, 144-146, 148,149, 150b, 150c, and 153-167, 294, 299, 300, 303, 307, 308, 310, 311, 312, 317, 319, 320, 324, 325, 327, 333, 336, and 337, and optionally one or more peptides comprising the amino acid sequences set forth in SEQ ID NO: 235-282. In some embodiments of the combination or pool of peptides of the disclosure, the combination or pool of peptides being characterized by the amino acid sequences set forth in SEQ ID NO: 174-175, 177a, 178, 179, 180a, 180b, 181, 182a, 182b, 183a, and 190, and 345-350, and optionally one or more peptides comprising the amino acid sequences set forth SEQ ID NO: 284-289.

In some embodiments of the combination or pool of peptides of the disclosure, the combination or pool of peptides being characterized by the amino acid sequences set forth in SEQ ID NO: 1-10, 12-14, 17, 18, 20-25, 27, 28, 30-39, 41, 43, 44 and 292, and optionally one or more peptides of SEQ ID NO: 11, 16, 19, 26, 29, 40, 42 and 45. In some embodiments of the combination or pool of peptides of the disclosure, the combination or pool of peptides being characterized by the amino acid sequences set forth in SEQ ID NO: 47, 48, 50-55, 59, 60, 62-64, 66-70, 72, 74, 75, 77, 79-81, 83, 84, 86, 87, 90-94, 96, 99-101, 103, 110, 115-125, 129, 131-133, 136-138, 140, 142, 144, 146, 148,149, 153-155, 158, 159, 294, 299, 300, 303, 308, 309, 311, 312, 317-321, 324, 325, 330-334, 337-340, and 354-357, and optionally one or more peptides of SEQ ID NO: 46, 47, 58, 61, 71, 76, 89, 105-109, 111, 113, 114, 126, 128, 130, 139, 141, 147, 151, 152, 160-167, and 265. In some embodiments of the combination or pool of peptides of the disclosure, the combination or pool of peptides being characterized by the amino acid sequences set forth in SEQ ID NO: 170, 175, 176, 178, 179, 181, 190, and 342-350, and optionally one or more peptides of SEQ ID NO: 168, 171, and 173. In some embodiments of the combination or pool of peptides of the disclosure, the combination or pool of peptides being characterized by the amino acid sequences set forth in SEQ ID NO: 235-248, 250, 252-255, 257-261, 268, 278-281, 290, 291, 351-353 and 358, and optionally one or more peptides of SEQ ID NO: 251, 266, 272, 274, 275, 277, 282 and 283. In some embodiments of the combination or pool of peptides of the disclosure, the combination or pool of peptides being characterized by the amino acid sequences set forth in SEQ ID NO: 293, 295-298, 301, 302, 304-306, 313-316, 322, 327 and 328.

### DETAILED DESCRIPTION OF THE DISCLOSURE

### General Techniques

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well-known and commonly used in the art. In case of conflict, the present specification, including definitions, will control.

The practice of the present disclosure will employ, unless otherwise indicated, conventional techniques of molecular biology (including recombinant techniques), microbiology, cell biology, biochemistry, virology and immunology, which are within the skill of the art. Such techniques are explained fully in the literature, such as Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, N.Y. (2001); Ausubel et al., Current Protocols in Molecular Biology, John Wiley & Sons, N Y (2002); Harlow and Lane Using Antibodies: A Laboratory Manual, Cold Spring Harbor Laboratory Press, Cold Spring Harbor, N.Y. (1998); Coligan et al., Short Protocols in Protein Science, John Wiley & Sons, N Y (2003); Short Protocols in Molecular Biology (Wiley and Sons, 1999).

Peptide synthesis reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well-known and commonly used in the art.

Throughout this specification and embodiments, the word “comprise,” or variations such as “comprises” or “comprising,” will be understood to imply the inclusion of a stated integer or group of integers, but not the exclusion of any other integer or group of integers. “Comprising” may be synonymous with “including” or “containing.”

It is understood that wherever embodiments are described herein with the language “comprising,” otherwise analogous embodiments described in terms of “consisting of” and/or “consisting essentially of” are also provided. As used herein, “consisting of” is a closed term that includes only the specific elements recited, and “consisting essentially of” includes the specific elements recited and may include additional unrecited, nonmaterial elements.

The term “including” is used to mean “including, but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a”, “an” and “the” are used herein to refer to one or to more than one (i.e., to at least one) of the grammatical object of the article. By way of example, “an element” means one element or more than one element. As used herein, the term “about” modifying the quantity of an ingredient, parameter, calculation, or measurement in the compositions of the disclosure or employed in the methods of the disclosure refers to variation in the numerical quantity that can occur, for example, through typical measuring and liquid handling procedures used for making isolated polypeptides or pharmaceutical compositions in the real world; through inadvertent error in these procedures; through differences in the manufacture, source, or purity of the ingredients employed to make the compositions or carry out the methods; and the like without having a substantial effect on the chemical or physical attributes of the compositions or methods of the disclosure. Such variation can be within an order of magnitude, typically within 10%, more typically still within 5%, of a given value or range. The term “about” also encompasses amounts that differ due to different equilibrium conditions for a composition resulting from a particular initial mixture. Whether or not modified by the term “about”, the paragraphs include equivalents to the quantities. Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

### Definitions

The following terms, unless otherwise indicated, shall be understood to have the following meanings:

As used herein, the terms “immune reaction” and “immune response” are used interchangeably herein and refer to the reaction of a subject's immune system to the presence of a substance which is not recognized as a constituent of the subject itself. An immune response may be a humoral immune response, a cell-mediated immune response, or a mixed humoral and cell-mediated immune response. A humoral response may be an antibody-mediated response. A cell-mediated response may be one or more of a cytotoxic T-cell mediated immune response, a macrophage mediated response, a natural killer (NK) cell mediated immune response or a cytokine mediated response. A mixed humoral and cell-mediated response may be one or more of an antibody-mediated response, a cytotoxic T-cell mediated immune response, a macrophage mediated response, a natural killer (NK) cell mediated immune response or a cytokine mediated response. The immune response can refer to an adaptive and/or an innate immune response. For the various types of immune responses, see David Chaplin (J Allergy Clin Immunol February; 125(2 Suppl 2): S3-23 (2010)). In some embodiments, the immune reaction corresponds to a delayed type hypersensitivity reaction (DTH) in the subject. See, e.g., Razzaque Ahmed et al.; Arch Dermatol. 1983; 119(11):934-945; incorporated by reference herein in its entirety.

The term “immunity”, as used herein, refers to the capability of a subject to resist invasive microorganisms or pathogens from entering its cells or replicating within the cells. Immunity involves both specific and non-specific components. The non-specific components act as barriers or eliminators of a wide range of pathogens irrespective of their antigenic make-up. Other components of the immune system adapt themselves to each new pathogen encountered and can generate pathogen-specific immunity.

The term “induration”, as used herein, refers to a localized hardening of soft tissue in the skin, caused by the swelling or inflammation of the skin.

The terms “cell-mediated immune response”, “cell-mediated immunity”, “T cell mediated immune response” and “T cell mediated immunity” are used interchangeably herein and refer to the primary response in a subject mainly against invasive microorganisms that cause intracellular infections. Naive T cells, which are immature T cells that have yet to encounter an antigen (such as the microorganism), are converted into activated effector T cells after encountering antigen-presenting cells (APCs). These APCs, such as macrophages, dendritic cells, and B cells in some circumstances, load antigenic peptides onto the MHC of the cell, in turn presenting the peptide to receptors on T cells. The cell-mediated immune response, therefore, involves the activation of phagocytes, antigen-specific cytotoxic T-lymphocytes (CD8⁺ cells) and the release of various cytokines in response to antigen. CD4⁺ cells or helper T cells are also activated by APC. The cell-mediated response may be Type IV hypersensitivity or Delayed Type Hypersensitivity (DTH). The DTH response is caused when CD4⁺ Th1 helper T cells recognize foreign antigen in a complex with the MHC class II on the surface of antigen-presenting cells. These can be macrophages that secrete IL-12, which stimulates the proliferation of further CD4⁺ Th1 cells. CD4⁺ T cells secrete IL-2 and interferon gamma, inducing the further release of other Th1 cytokines, thus mediating the immune response. Activated CD8⁺ T cells destroy target cells on contact, whereas activated macrophages produce hydrolytic enzymes and, on presentation with certain intracellular pathogens, transform into multinucleated giant cells.

The term “intradermal application” or “intradermal administration”, as used herein, refers to the administration of a composition comprising the one or more peptides of the disclosure to the epidermis or dermis layer. It includes administrations where the composition is delivered directly to the dermis (e.g. by a device which passes entirely through the epidermis to the dermis) and those where the composition is first delivered into the epidermis by penetration of the epidermis, wherein the composition then moves through the epidermis to the dermis.

The terms “patient”, “subject”, or “individual” are used interchangeably herein and refer to either a human or a non-human animal. These terms include mammals, such as humans, primates, livestock animals (including bovines, porcines, camels, etc.), companion animals (e.g., canines, felines, etc.), animals present in a zoo and rodents (e.g., mice and rats).

The terms “polypeptide” and “protein” are used interchangeably and refer to chains of amino acids of greater than about 50 amino acids. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

The terms “oligopeptide” and “peptide” are used interchangeably and refer to a sequence of amino acids made up of a single chain of amino acids joined by peptide bonds. In some embodiments, peptides contain between 2-30 amino acids in length, unless otherwise defined. In some embodiments, peptides contain between 2-50 amino acids in length, unless otherwise defined. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, peptides containing one or more analogs of an amino acid (including, for example, unnatural amino acids, etc.), as well as other modifications known in the art.

As used herein, “purify,” and grammatical variations thereof, refers to the removal, whether completely or partially, of at least one impurity from a mixture containing the polypeptide and one or more impurities, which thereby improves the level of purity of the polypeptide of the disclosure (i.e., by decreasing the amount (ppm) of impurity(ies) in the composition). A peptide can be “purified” using routine methods known to one of skill in the art including, but not limited to, chromatography.

As used herein, the term “residue” in the context of a polypeptide or peptide refers to an amino-acid unit in the linear polypeptide or peptide chain. It is what remains of each amino acid, i.e —NH—CHR—C—, after water is removed in the formation of the polypeptide from α-amino-acids, i.e. NH2-CHR—COOH.

The term “sequence identity,” in all its grammatical forms, refers to the degree of identity or correspondence between nucleic acid or amino acid sequences that may or may not share a common evolutionary origin.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

The term “vaccine” or “vaccination” to SARS-CoV-2, as used herein, refers to a composition comprising at least one immunologically active component that is potentially or is capable of inducing an immunological response in an animal to SARS-CoV-2; and possibly, but not necessarily, comprises one or more additional components that enhance the immunological activity of the active component. A vaccine may additionally comprise further components typical to pharmaceutical compositions. The immunologically active component of a vaccine to SARS-CoV-2 may comprise, for example, complete virus particles in either their original form or as attenuated particles (modified live vaccine), or particles inactivated by appropriate methods (killed or inactivated vaccine). In other embodiments, the immunologically active component of a vaccine may comprise appropriate elements of the organisms (subunit vaccines) that are capable of stimulating the immune system. The immunologically active component may be a protein of the viral envelope. The immunologically active component may comprise a protein forming part of the nucleocapsid. In some embodiments, the immunologically active component of a vaccine against SARS-CoV-2 comprises a structural protein (e.g., the Spike protein (S), the Membrane protein (M), the Nucleocapsid protein (N) and the Envelope protein (E)).

As used herein, the term “variant” refers to a polypeptide or peptide having a substantial sequence identity to a reference polypeptide or peptide. A variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

As used herein, the term “superantigen region”, also referred to as “superantigen domain,” refers to a sequence (e.g., peptide sequence) that potentially engages a T cell receptor and elicits a T cell response.

Each embodiment described herein may be used individually or in combination with any other embodiment described herein.

### SARS-CoV-2 Peptides

In one aspect, the present disclosure relates to a method for detecting SARS-CoV-2 cell-mediated immunity in a subject, a method for detecting a cell-mediated immune response to SARS-CoV-2 in a subject, a method for determining if a potential or approved vaccine to SARS-CoV-2 elicits a cell-mediated immune response in a subject or a method for detecting a SARS-CoV-2 infection in a subject, the methods comprising administering to the skin of the subject one or more of the peptides of the disclosure.

In one aspect, the present disclosure relates to a method for detecting SARS-CoV-2 cell-mediated immunity in a subject, a method for detecting a cell-mediated immune response to SARS-CoV-2 in a subject, a method for determining if a potential or approved vaccine to SARS-CoV-2 elicits a cell-mediated immune response in a subject, a method for detecting a SARS-CoV-2 infection in a subject, a method to stratify participants in a SARS-CoV-2 vaccine trials or to assess surrogate markers in those trials to evaluate cMI responses to the vaccine or to assess the durability of a CMI response over time, the methods comprising administering to the skin of the subject one or more of the peptides of the disclosure, and preferably various combinations of those peptides.

In some embodiments, the one, five, ten, fifteen, twenty, twenty-five, thirty or more peptides used in the methods of the disclosure are selected from the group consisting of peptides being characterized by the amino acid sequences set forth in SEQ ID NO: 1-183. In some embodiments, at least one, at least five, at least ten, at least fifteen, at least twenty, at least twenty-five, at least thirty of the one or more peptides used in the methods of the disclosure is selected from the sequences set forth in SEQ ID NO: 1-183. In some embodiments, at least one, at least five, at least ten, at least fifteen, at least twenty, at least twenty-five, at least thirty of the one or more peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequence of the corresponding peptides set forth in SEQ ID NO: 1-183. In some embodiments, at least two or more of the peptides being characterized by the amino acid sequence set forth in SEQ ID NO: 1-183 are fused into a single polypeptide. In some embodiments, at least one, at least five, at least ten, at least fifteen, at least twenty, at least twenty-five, at least thirty or more of the one or more peptides being characterized by the amino acid sequence set forth in SEQ ID NO: 1-183 are individual peptides. In some embodiments, at least one, at least five, at least ten, at least fifteen, at least twenty, at least twenty-five, at least thirty or more of the one or more peptides being characterized by the amino acid sequence set forth in SEQ ID NO: 1-183 are individual peptides and preferably combinations of those individual peptides.

In some embodiments, the one, five, ten, fifteen, twenty, thirty or more peptides used in the methods of the disclosure are selected from the group consisting of peptides being characterized by the amino acid sequences set forth in SEQ ID NO: 1-183 and 189-231. In some embodiments, at least one, at least five, at least ten, at least fifteen, at least twenty, at least twenty-five, at least thirty of the one or more peptides used in the methods of the disclosure is selected from the sequences set forth in SEQ ID NO: 1-183 and 189-231. In some embodiments, at least one, at least five, at least ten, at least fifteen, at least twenty, at least twenty-five, at least thirty of the one or more peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequence of the corresponding peptides set forth in SEQ ID NO: 1-183 and 189-231. In some embodiments, at least two or more of the peptides being characterized by the amino acid sequence set forth in SEQ ID NO: 1-183 and 189-231 are fused into a single polypeptide. In some embodiments, at least one, at least five, at least ten, at least fifteen, at least twenty, at least twenty-five, at least thirty or more of the one or more peptides being characterized by the amino acid sequence set forth in SEQ ID NO: 1-183 and 189-231 are individual peptides. In some embodiments, at least one, at least five, at least ten, at least fifteen, at least twenty, at least twenty-five, at least thirty or more of the one or more peptides being characterized by the amino acid sequence set forth in SEQ ID NO: 1-183 and 189-231 are individual peptides and combinations of the individual peptides.

In some embodiments, the one, five, ten, fifteen, twenty, thirty or more of peptides used in the methods of the disclosure are selected from the group consisting of peptides being characterized by the amino acid sequences set forth in SEQ ID NO: 1-358. In some embodiments, at least one, at least five, at least ten, at least fifteen, at least twenty, at least twenty-five, at least thirty of the one or more peptides used in the methods of the disclosure is selected from the sequences set forth in SEQ ID NO: 1-358. In some embodiments, at least one, at least five, at least ten, at least fifteen, at least twenty, at least twenty-five, at least thirty of the one or more peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequence of the corresponding peptides set forth in SEQ ID NO: 1-183 and 189-231. In some embodiments, at least two or more of the peptides being characterized by the amino acid sequence set forth in SEQ ID NO: 1-358 are fused into a single polypeptide. In some embodiments, at least one, at least five, at least ten, at least fifteen, at least twenty, at least twenty-five, at least thirty or more of the one or more peptides being characterized by the amino acid sequence set forth in SEQ ID NO: 1-358 are individual peptides. In some embodiments, at least one, at least five, at least ten, at least fifteen, at least twenty, at least twenty-five, at least thirty or more of the one or more peptides being characterized by the amino acid sequence set forth in SEQ ID NO: 1-358 are individual peptides and combinations of the individual peptides.

In some embodiments, the one, five, ten, fifteen, twenty, twenty-five, thirty or more of peptides used in the methods of the disclosure are selected from the group consisting of peptides being characterized by the amino acid sequences set forth in SEQ ID NO: 1-183, 189-231 and 234-358. In some embodiments, at least one, at least five, at least ten, at least fifteen, at least twenty, at least twenty-five, at least thirty of the one or more peptides used in the methods of the disclosure is selected from the sequences set forth in SEQ ID NO: 1-183, 189-231 and 234-358. In some embodiments, at least one, at least five, at least ten, at least fifteen, at least twenty, at least twenty-five, at least thirty of the one or more peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequence of the corresponding peptides set forth in SEQ ID NO: 1-183, 189-231 and 234-358. In some embodiments, at least two or more of the peptides being characterized by the amino acid sequence set forth in SEQ ID NO: 1-183, 189-231 and 234-358 are fused into a single polypeptide. In some embodiments, at least one, at least five, at least ten, at least fifteen, at least twenty, at least twenty-five, at least thirty or more of the one or more peptides being characterized by the amino acid sequence set forth in SEQ ID NO: 1-183, 189-231 and 234-358 are individual peptides. In some embodiments, at least one, at least five, at least ten, at least fifteen, at least twenty, at least twenty-five, at least thirty or more of the one or more peptides being characterized by the amino acid sequence set forth in SEQ ID NO: 1-183, 189-231 and 234-358 are individual peptides and combinations of the individual peptides. In some embodiments, at least one, at least five, at least ten, at least fifteen, at least twenty, at least twenty-five, at least thirty of the one or more peptides used in the methods of the disclosure is selected from the sequences set forth in SEQ ID NO: 1-183, 189-231 and 234-358.

In some embodiments, the one, five, ten, fifteen, twenty, twenty-five, thirty or more peptides used in the methods of the disclosure are selected from the group consisting of peptides being characterized by the amino acid sequences set forth in SEQ ID NO: 1-183, 189-232, 294, 299, 300, 303, 307-312, 317, 319, 320, 324-327, 333 and 335-337, 341 and 342. In some embodiments, at least one, at least five, at least ten, at least fifteen, at least twenty, at least twenty-five, at least thirty of the one or more peptides used in the methods of the disclosure is selected from the sequences set forth in SEQ ID NO: 1-183, 189-232, 294, 299, 300, 303, 307-312, 317, 319, 320, 324-327, 333 and 335-337, 341 and 342. In some embodiments, at least one, at least five, at least ten, at least fifteen, at least twenty, at least twenty-five, at least thirty of the one or more peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequence of the corresponding peptides set forth in SEQ ID NO: 1-183 and 189-231. In some embodiments, at least two or more of the peptides being characterized by the amino acid sequence set forth in SEQ ID NO: 1-183, 189-232, 294, 299, 300, 303, 307-312, 317, 319, 320, 324-327, 333 and 335-337, 341 and 342 are fused into a single polypeptide. In some embodiments, at least one, at least five, at least ten, at least fifteen, at least twenty, at least twenty-five, at least thirty or more of the one or more peptides being characterized by the amino acid sequence set forth in SEQ ID NO: 1-183, 189-232, 294, 299, 300, 303, 307-312, 317, 319, 320, 324-327, 333 and 335-337, 341 and 342 are individual peptides. In some embodiments, at least one, at least five, at least ten, at least fifteen, at least twenty, at least twenty-five, at least thirty or more of the one or more peptides being characterized by the amino acid sequence set forth in SEQ ID NO: 1-183, 189-232, 294, 299, 300, 303, 307-312, 317, 319, 320, 324-327, 333 and 335-337, 341 and 342 are individual peptides and combinations of the individual peptides. In some embodiments, the one, five, ten, fifteen, twenty, twenty-five, thirty or more peptides used in the methods of the disclosure are selected from the group consisting of peptides being characterized by the amino acid sequences set forth in SEQ ID NO: 1-183, 189-232, 294, 299, 300, 303, 307-312, 317, 319, 320, 324-327, 333 and 335-337, 341 and 342. In some embodiments, at least one, at least five, at least ten, at least fifteen, at least twenty, at least twenty-five, at least thirty or more of the peptides being characterized by the amino acid sequence set forth in SEQ ID NO: 1-183, 189-232, 294, 299, 300, 303, 307-312, 317, 319, 320, 324-327, 333 and 335-337, 341 and 342 are individual peptides and combinations of the individual peptides.

In some embodiments, the one, five, ten, fifteen, twenty, twenty-five, thirty or more peptides used in the methods of the disclosure are selected from the group consisting of peptides being characterized by the amino acid sequences set forth in SEQ ID NO: 1-104, 109-138, 140-183, 189-231, 235-289, 294, 299, 300, 303, 307-312, 317, 319, 320, 324-327, 333, 335-337, 341, 342 and 345-350. In some embodiments, at least one, at least five, at least ten, at least fifteen, at least twenty, at least twenty-five, at least thirty of the one or more peptides used in the methods of the disclosure is selected from the sequences set forth in SEQ ID NO: 1-104, 109-138, 140-183, 189-231, 235-289, 294, 299, 300, 303, 307-312, 317, 319, 320, 324-327, 333, 335-337, 341, 342 and 345-350. In some embodiments, at least, at least five, at least ten, at least fifteen, at least twenty, at least twenty-five, at least thirty one of the one or more peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequence of the corresponding peptides set forth in SEQ ID NO: 1-104, 109-138, 140-183, 189-231, 235-289, 294, 299, 300, 303, 307-312, 317, 319, 320, 324-327, 333, 335-337, 341, 342 and 345-350. In some embodiments, at least two or more of the peptides being characterized by the amino acid sequence set forth in SEQ ID NO: 1-104, 109-138, 140-183, 189-231, 235-289, 294, 299, 300, 303, 307-312, 317, 319, 320, 324-327, 333, 335-337, 341, 342 and 345-350 are fused into a single polypeptide. In some embodiments, at least one, at least five, at least ten, at least fifteen, at least twenty, at least twenty-five, at least thirty or more of the one or more peptides being characterized by the amino acid sequence set forth in SEQ ID NO: 1-104, 109-138, 140-183, 189-231, 235-289, 294, 299, 300, 303, 307-312, 317, 319, 320, 324-327, 333, 335-337, 341, 342 and 345-350 are individual peptides. In some embodiments, at least one, at least five, at least ten, at least fifteen, at least twenty, at least twenty-five, at least thirty or more of the one or more peptides being characterized by the amino acid sequence set forth in SEQ ID NO: 1-104, 109-138, 140-183, 189-231, 235-289, 294, 299, 300, 303, 307-312, 317, 319, 320, 324-327, 333, 335-337, 341, 342 and 345-350 are individual peptides and combinations of the individual peptides.

In some embodiments, the one, five, ten, fifteen, twenty, twenty-five, thirty or more peptides used in the methods of the disclosure are selected from the group consisting of peptides being characterized by the amino acid sequences set forth in SEQ ID NO: 1-183, 189-231 and 234-358. In some embodiments, at least one, at least five, at least ten, at least fifteen, at least twenty, at least twenty-five, at least thirty or more of the one or more peptides used in the methods of the disclosure is selected from the sequences set forth in SEQ ID NO: 1-183, 189-231 and 234-358. In some embodiments, at least one of the one or more peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequence of the corresponding peptides set forth in SEQ ID NO: 1-183, 189-231 and 234-358. In some embodiments, at least two or more of the peptides being characterized by the amino acid sequence set forth in SEQ ID NO: 1-183, 189-231 and 234-358 are fused into a single polypeptide. In some embodiments, at least one or more of the peptides being characterized by the amino acid sequence set forth in SEQ ID NO: 1-183, 189-231 and 234-358 are individual peptides. In some embodiments, at least one, at least five, at least ten, at least fifteen, at least twenty, at least twenty-five, at least thirty or more of the peptides being characterized by the amino acid sequence set forth in SEQ ID NO: 1-183, 189-231 and 234-358 are individual peptides and combinations of the individual peptides.

In some embodiments, the one, five, ten, fifteen, twenty, twenty-five, thirty or more peptides used in the methods of the disclosure are selected from the group consisting of peptides being characterized by the amino acid sequences set forth in SEQ ID NO: 1-14, 16-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-168, 170, 171, 173, 175, 176, 178, 179, 181, 190, 235-248, 250-255, 257-261, 266, 268, 265, 272, 274, 275, 277-283, 290-306, 308-322, 324, 325, 328, 328, 330-334, 337-340, and 342-358. In some embodiments, at least one, at least five, at least ten, at least fifteen, at least twenty, at least twenty-five, at least thirty of the one or more peptides used in the methods of the disclosure is selected from the sequences set forth in SEQ ID NO: 1-14, 16-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-168, 170, 171, 173, 175, 176, 178, 179, 181, 190, 235-248, 250-255, 257-261, 266, 268, 265, 272, 274, 275, 277-283, 290-306, 308-322, 324, 325, 328, 328, 330-334, 337-340, and 342-358. In some embodiments, at least one, at least five, at least ten, at least fifteen, at least twenty, at least twenty-five, at least thirty of the one or more peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequence of the corresponding peptides set forth in SEQ ID NO: 1-14, 16-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-168, 170, 171, 173, 175, 176, 178, 179, 181, 190, 235-248, 250-255, 257-261, 266, 268, 265, 272, 274, 275, 277-283, 290-306, 308-322, 324, 325, 328, 328, 330-334, 337-340, and 342-358. In some embodiments, at least two or more of the peptides being characterized by the amino acid sequence set forth in SEQ ID NO: 1-14, 16-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-168, 170, 171, 173, 175, 176, 178, 179, 181, 190, 235-248, 250-255, 257-261, 266, 268, 265, 272, 274, 275, 277-283, 290-306, 308-322, 324, 325, 328, 328, 330-334, 337-340, and 342-358 are fused into a single polypeptide. In some embodiments, at least one, at least five, at least ten, at least fifteen, at least twenty, at least twenty-five, at least thirty or more of the peptides being characterized by the amino acid sequence set forth in SEQ ID NO: 1-14, 16-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-168, 170, 171, 173, 175, 176, 178, 179, 181, 190, 235-248, 250-255, 257-261, 266, 268, 265, 272, 274, 275, 277-283, 290-306, 308-322, 324, 325, 328, 328, 330-334, 337-340, and 342-358 are individual peptides. In some embodiments, at least one, at least five, at least ten, at least fifteen, at least twenty, at least twenty-five, at least thirty or more of the one or more peptides being characterized by the amino acid sequence set forth in SEQ ID NO: 1-14, 16-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-168, 170, 171, 173, 175, 176, 178, 179, 181, 190, 235-248, 250-255, 257-261, 266, 268, 265, 272, 274, 275, 277-283, 290-306, 308-322, 324, 325, 328, 328, 330-334, 337-340, and 342-358 are individual peptides and combinations of the individual peptides.

In some embodiments, the one or more peptides used in the methods of the disclosure are selected from the group consisting of peptides being characterized by any one of the amino acid sequences set forth in SEQ ID NO: 1-45. In some embodiments, at least one of the one or more peptides used in the methods of the disclosure is selected from any one of the sequences set forth in SEQ ID NO: 1-45. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 1-45. In some embodiments, the one, five, ten, fifteen, twenty, twenty-five or thirty or more peptides used in the methods of the disclosure are selected from the group consisting of peptides being characterized by any one of the amino acid sequences set forth in SEQ ID NO: 1-45. In some embodiments, at least one, at least five, at least ten, at least fifteen, at least twenty, at least twenty-five, at least thirty or more of the one or more peptides used in the methods of the disclosure is selected from any one of the sequences set forth in SEQ ID NO: 1-45. In some embodiments, at least one, at least five, at least ten, at least fifteen, at least twenty, at least twenty-five, at least thirty or more of the one or more peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 1-45.

In some embodiments, the one or more peptides used in the methods of the disclosure are selected from the group consisting of peptides being characterized by any one of the amino acid sequences set forth in SEQ ID NO: 1-10, 12-25, 27, 28, 30-39, 41, 43 and 44. In some embodiments, at least one of the one or more peptides used in the methods of the disclosure is selected from any one of the sequences set forth in SEQ ID NO: 1-10, 12-25, 27, 28, 30-39, 41, 43 and 44. In some embodiments, at least one or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 1-10, 12-25, 27, 28, 30-39, 41, 43 and 44. In some embodiments, the one, five, ten, fifteen, twenty, thirty or more peptides used in the methods of the disclosure are selected from the group consisting of peptides being characterized by any one of the amino acid sequences set forth in SEQ ID NO: 1-10, 12-25, 27, 28, 30-39, 41, 43 and 44. In some embodiments, at least one, at least five, at least ten, at least fifteen, at least twenty, at least twenty-five, at least thirty of the one or more peptides used in the methods of the disclosure is selected from any one of the sequences set forth in SEQ ID NO: 1-10, 12-25, 27, 28, 30-39, 41, 43 and 44. In some embodiments, at least one, at least five, at least ten, at least fifteen, at least twenty, at least twenty-five, at least thirty or more of the one or more peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 1-10, 12-25, 27, 28, 30-39, 41, 43 and 44.

In some embodiments, the one or more peptides used in the methods of the disclosure are selected from the group consisting of peptides being characterized by any one of the amino acid sequences set forth in SEQ ID NO: 1-23. In some embodiments, at least one of the one or more peptides used in the methods of the disclosure is selected from any one of the sequences set forth in SEQ ID NO: 1-23. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 1-23. In some embodiments, the one, five, ten, fifteen, twenty or more peptides used in the methods of the disclosure are selected from the group consisting of peptides being characterized by any one of the amino acid sequences set forth in SEQ ID NO: 1-23. In some embodiments, at least one, at least five, at least ten, at least fifteen, at least twenty of the one or more peptides used in the methods of the disclosure is selected from any one of the sequences set forth in SEQ ID NO: 1-23. In some embodiments, at least one, at least five, at least ten, at least fifteen, at least twenty or more of the one or more peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 1-23.

In some embodiments, the one, five, ten, fifteen or more peptides used in the methods of the disclosure are selected from the group consisting of peptides being characterized by any one of the amino acid sequences set forth in SEQ ID NO: 1-10 and 12-23. In some embodiments, at least one, at least five, at least ten, at least fifteen of the one or more peptides used in the methods of the disclosure is selected from any one of the sequences set forth in SEQ ID NO: 1-10 and 12-23. In some embodiments, at least one, at least five, at least ten, at least fifteen or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 1-10 and 12-23.

In some embodiments, the one, five, ten, fifteen, twenty, twenty-five, thirty or more peptides used in the methods of the disclosure are selected from the group consisting of peptides being characterized by any one of the amino acid sequences set forth in SEQ ID NO: 1-14, 16-45 and 292. In some embodiments, at least one, at least five, at least ten, at least fifteen, at least twenty, at least twenty-five, at least thirty of the one or more peptides used in the methods of the disclosure is selected from any one of the sequences set forth in SEQ ID NO: 1-14, 16-45 and 292. In some embodiments, at least one or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 1-14, 16-45 and 292.

In some embodiments, the one, five, ten, fifteen, twenty, thirty or more peptides used in the methods of the disclosure are selected from the group consisting of peptides being characterized by any one of the amino acid sequences set forth in SEQ ID NO: 1-14, 16-45 and 292. In some embodiments, at least one, at least five, at least ten, at least fifteen, at least twenty, at least twenty-five, at least thirty of the one or more peptides used in the methods of the disclosure is selected from any one of the sequences set forth in SEQ ID NO: 1-10, 12-14, 17, 18, 20-25, 27, 28, 30-39, 41, 43, 44 and 292, and optionally one or more sequences characterized by the amino acid sequences set forth in SEQ ID NO: 1-10, 12-14, 17, 18, 20-25, 27, 28, 30-39, 41, 43, 44 and 292, and optionally one or more sequences characterized by the amino acid sequences set forth in SEQ ID NO: 11, 16, 19, 26, 29, 40, 42, and 45. In some embodiments, at least one, at least five, at least ten, at least fifteen, at least twenty, at least twenty-five, at least thirty or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 1-10, 12-14, 17, 18, 20-25, 27, 28, 30-39, 41, 43, 44 and 292, and optionally one or more sequences characterized by the amino acid sequences set forth in SEQ ID NO: 11, 16, 19, 26, 29, 40, 42, and 45.

In some embodiments, the one or more peptides used in the methods of the disclosure are selected from the group consisting of peptides being characterized by any one of the amino acid sequences set forth in SEQ ID NO: 26-28. In some embodiments, at least one of the one or more peptides used in the methods of the disclosure is selected from any one of the sequences set forth in SEQ ID NO: 26-28. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 26-28.

In some embodiments, the one or more peptides used in the methods of the disclosure are selected from the group consisting of peptides being characterized by any one of the amino acid sequences set forth in SEQ ID NO: 27 and 28. In some embodiments, at least one of the one or more peptides used in the methods of the disclosure is selected from any one of the sequences set forth in SEQ ID NO: 27 and 28. In some embodiments, at least one or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 27 and 28.

In some embodiments, the one or more peptides used in the methods of the disclosure are selected from the group consisting of peptides being characterized by any one of the amino acid sequences set forth in SEQ ID NO: 29-38. In some embodiments, at least one of the one or more peptides used in the methods of the disclosure is selected from any one of the sequences set forth in SEQ ID NO: 29-38. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 29-38.

In some embodiments, the one, five or more peptides used in the methods of the disclosure are selected from the group consisting of peptides being characterized by any one of the amino acid sequences set forth in SEQ ID NO: 30-38. In some embodiments, at least one, at least five or more of the one or more peptides used in the methods of the disclosure is selected from any one of the sequences set forth in SEQ ID NO: 30-38. In some embodiments, at least one, at least five or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 30-38.

In some embodiments, the one, five or more of the one or more peptides used in the methods of the disclosure are selected from the group consisting of peptides being characterized by any one of the amino acid sequences set forth in SEQ ID NO: 24-25 and SEQ ID NO: 39-45. In some embodiments, at least one, at least five or more of the one or more peptides used in the methods of the disclosure is selected from any one of the sequences set forth in SEQ ID NO: 24-25 and SEQ ID NO: 39-45. In some embodiments, at least one, at least five or more of the one or more peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 24-25 and SEQ ID NO: 39-45.

In some embodiments, the one or more peptides used in the methods of the disclosure are selected from the group consisting of peptides being characterized by any one of the amino acid sequences set forth in SEQ ID NO: 24-25 and SEQ ID NO: 29, 41, 43 and 44. In some embodiments, at least one of the one or more peptides used in the methods of the disclosure is selected from any one of the sequences set forth in SEQ ID NO: 24-25 and SEQ ID NO: 29, 41, 43 and 44. In some embodiments, at least one or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 24-25 and SEQ ID NO: 29, 41, 43 and 44.

In some embodiments, the one, five, ten, fifteen, twenty, twenty-five, thirty or more peptides used in the methods of the disclosure are selected from the group consisting of peptides being characterized by any one of the amino acid sequences set forth in SEQ ID NO: 24-25 and SEQ ID NO: 39-45 and 194-231. In some embodiments, at least one, at least five, at least ten, at least fifteen, at least twenty, at least twenty-five, at least thirty of the one or more peptides used in the methods of the disclosure is selected from any one of the sequences set forth in SEQ ID NO: 24-25 and SEQ ID NO: 39-45 and 194-231. In some embodiments, at least one, at least five, at least ten, at least fifteen, at least twenty, at least twenty-five, at least thirty or more of the one or more peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 24-25 and SEQ ID NO: 39-45 and 194-231.

In some embodiments, the one or more peptides used in the methods of the disclosure are selected from the group consisting of peptides being characterized by any one of the amino acid sequences set forth in SEQ ID NO: 24, 25, 41, 43, 44, and optionally one or more sequences characterized by the amino acid sequences set forth in SEQ ID NO: 40, 42 and 45. In some embodiments, at least one of the one or more peptides used in the methods of the disclosure is selected from any one of the sequences set forth in SEQ ID NO: 24, 25, 41, 43, 44, and optionally one or more sequences characterized by the amino acid sequences set forth in SEQ ID NO: 40, 42 and 45. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 24, 25, 41, 43, 44, and optionally one or more sequences characterized by the amino acid sequences set forth in SEQ ID NO: 40, 42 and 45.

In some embodiments, the one, five, ten, fifteen, twenty, twenty-five, thirty or more peptides used in the methods of the disclosure are selected from the group consisting of peptides being characterized by any one of the amino acid sequences set forth in SEQ ID NO: 194-231. In some embodiments, at least one, at least five, at least ten, at least fifteen, at least twenty, at least twenty-five, at least thirty of the one or more peptides used in the methods of the disclosure is selected from any one of the sequences set forth in SEQ ID NO: 194-231. In some embodiments, at least one, at least five, at least ten, at least fifteen, at least twenty, at least twenty-five, at least thirty or more of the one or more peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 194-231.

In some embodiments, the one, five, ten, fifteen, twenty, twenty-five, thirty or more peptides used in the methods of the disclosure are selected from the group consisting of peptides being characterized by the amino acid sequences set forth in SEQ ID NO: 46-167. In some embodiments, at least one, at least five, at least ten, at least fifteen, at least twenty, at least twenty-five, at least thirty of the one or more peptides used in the methods of the disclosure is selected from the sequences set forth in SEQ ID NO: 46-167. In some embodiments, at least one, at least five, at least ten, at least fifteen, at least twenty, at least twenty-five, at least thirty or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 46-167.

In some embodiments, the one, five, ten, fifteen, twenty, twenty-five, thirty or more peptides used in the methods of the disclosure are selected from the group consisting of peptides being characterized by the amino acid sequences set forth in SEQ ID NO: 46-70, 72-94, 96, 97, 99-101, 103-107, 109-111, 113-149, 151-167, 307, 317, 319, 320, 323, 324 and 335. In some embodiments, at least one, at least five, at least ten, at least fifteen, at least twenty, at least twenty-five, at least thirty of the one or more peptides used in the methods of the disclosure is selected from the sequences set forth in SEQ ID NO: 46-70, 72-94, 96, 97, 99-101, 103-107, 109-111, 113-149, 151-167, 307, 317, 319, 320, 323, 324 and 335. In some embodiments, at least one, at least five, at least ten, at least fifteen, at least twenty, at least twenty-five, at least thirty or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 46-70, 72-94, 96, 97, 99-101, 103-107, 109-111, 113-149, 151-167, 307, 317, 319, 320, 323, 324 and 335.

In some embodiments, the one, five, ten, fifteen, twenty, twenty-five, thirty or more peptides used in the methods of the disclosure are selected from the group consisting of peptides being characterized by the amino acid sequences set forth in SEQ ID NO: 47, 50-55, 59, 60, 62-64, 66-70, 72, 74-77, 79-81, 83, 84, 86, 87, 90-94, 96, 97, 99-101, 103, 110, 114-126, 129, 131-134, 135-138, 140, 144-146, 148,149, 153-167, 294, 299, 300, 303, 307, 308, 310-312, 317, 319, 320, 324, 325, 327, 333, 336 and 337, and optionally one or more peptides being characterized by the amino acid sequences set forth in SEQ ID NO: 235-282. In some embodiments, at least one, at least five, at least ten, at least fifteen, at least twenty, at least twenty-five, at least thirty of the one or more peptides used in the methods of the disclosure is selected from the sequences set forth in SEQ ID NO: 47, 50-55, 59, 60, 62-64, 66-70, 72, 74-77, 79-81, 83, 84, 86, 87, 90-94, 95a, 96, 97, 98a, 99-101, 102a, 103, 110, 112a, 114-126, 127a, 129, 131-134, 135-138, 140, 144-146, 148,149, 153-167, 294, 299, 300, 303, 307, 308, 310-312, 317, 319, 320, 324, 325, 327, 333, 336 and 337, and optionally one or more peptides being characterized by the amino acid sequences set forth in SEQ ID NO: 235-282. In some embodiments, at least one, at least five, at least ten, at least fifteen, at least twenty, at least twenty-five, at least thirty or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 47, 50-55, 59, 60, 62-64, 66-70, 72, 74-77, 79-81, 83, 84, 86, 87, 90-94, 96, 97, 99-101, 103, 110, 114-126, 129, 131-134, 135-138, 140, 144-146, 148,149, 153-167, 294, 299, 300, 303, 307, 308, 310-312, 317, 319, 320, 324, 325, 327, 333, 336 and 337, and optionally one or more peptides being characterized by the amino acid sequences set forth in SEQ ID NO: 235-282.

In some embodiments, the one, five, ten, fifteen, twenty, twenty-five, thirty or more peptides used in the methods of the disclosure are selected from the group consisting of peptides being characterized by the amino acid sequences set forth in SEQ ID NO: 46-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-167, 265, 294, 299, 300, 303, 308, 309, 311, 312, 317-321, 324, 325, 330-334, 337-340, and 354-357. In some embodiments, at least one, at least five, at least ten, at least fifteen, at least twenty, at least twenty-five, at least thirty of the one or more peptides used in the methods of the disclosure is selected from the sequences set forth in SEQ ID NO: 46-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-167, 265, 294, 299, 300, 303, 308, 309, 311, 312, 317-321, 324, 325, 330-334, 337-340, and 354-357. In some embodiments, at least one, at least five, at least ten, at least fifteen, at least twenty, at least twenty-five, at least thirty or more of the one or more peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 46-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-167, 265, 294, 299, 300, 303, 308, 309, 311, 312, 317-321, 324, 325, 330-334, 337-340, and 354-357.

In some embodiments, the one, five, ten, fifteen, twenty, twenty-five, thirty or more peptides used in the methods of the disclosure are selected from the group consisting of peptides being characterized by the amino acid sequences set forth in SEQ ID NO: 47, 48, 50-55, 59, 60, 62-64, 66-70, 72, 74, 75, 77, 79-81, 83, 84, 86, 87, 90-94, 96, 99-101, 103, 110, 115-125, 129, 131-133, 136-138, 140, 142, 144, 146, 148,149, 153-155, 158, 159, 294, 299, 300, 303, 308, 309, 311, 312, 317-321, 324, 325, 330-334, 337-340, and 354-357, and optionally one or more peptides being characterized by the amino acid sequences set forth in SEQ ID NO: 46, 47, 58, 61, 71, 76, 89, 105-109, 111, 113, 114, 126, 128, 130, 139, 141, 147, 151, 152, 160-167, and 265. In some embodiments, at least one, at least five, at least ten, at least fifteen, at least twenty, at least twenty-five, at least thirty of the one or more peptides used in the methods of the disclosure is selected from the sequences set forth in SEQ ID NO: 47, 48, 50-55, 59, 60, 62-64, 66-70, 72, 74, 75, 77, 79-81, 83, 84, 86, 87, 90-94, 96, 99-101, 103, 110, 115-125, 129, 131-133, 136-138, 140, 142, 144, 146, 148,149, 153-155, 158, 159, 294, 299, 300, 303, 308, 309, 311, 312, 317-321, 324, 325, 330-334, 337-340, and 354-357, and optionally one or more peptides being characterized by the amino acid sequences set forth in SEQ ID NO: 46, 47, 58, 61, 71, 76, 89, 105-109, 111, 113, 114, 126, 128, 130, 139, 141, 147, 151, 152, 160-167, and 265. In some embodiments, at least one, at least five, at least ten, at least fifteen, at least twenty, at least twenty-five, at least thirty or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 47, 48, 50-55, 59, 60, 62-64, 66-70, 72, 74, 75, 77, 79-81, 83, 84, 86, 87, 90-94, 96, 99-101, 103, 110, 115-125, 129, 131-133, 136-138, 140, 142, 144, 146, 148,149, 153-155, 158, 159, 294, 299, 300, 303, 308, 309, 311, 312, 317-321, 324, 325, 330-334, 337-340, and 354-357, and optionally one or more peptides being characterized by the amino acid sequences set forth in SEQ ID NO: 46, 47, 58, 61, 71, 76, 89, 105-109, 111, 113, 114, 126, 128, 130, 139, 141, 147, 151, 152, 160-167, and 265.

In some embodiments, the one, five, ten or more peptides used in the methods of the disclosure are selected from the group consisting of peptides being characterized by the amino acid sequences set forth in SEQ ID NO: 168-183. In some embodiments, at least one, at least five, at least ten of the one or more peptides used in the methods of the disclosure is selected from any one of the sequences set forth in SEQ ID NO: 168-183. In some embodiments, at least one, at least five, at least ten or more of the one or more peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 168-183.

In some embodiments, the one, five, ten or more peptides used in the methods of the disclosure are selected from the group consisting of peptides being characterized by the amino acid sequences set forth in SEQ ID NO: 168-183, 189 and 190. In some embodiments, at least one, at least five, at least ten of the one or more peptides used in the methods of the disclosure is selected from any one of the sequences set forth in SEQ ID NO: 168-183, 189 and 190. In some embodiments, at least one, at least five, at least ten or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 168-183, 189 and 190.

In some embodiments, the one or more peptides used in the methods of the disclosure are selected from the group consisting of peptides being characterized by the amino acid sequences set forth in SEQ ID NO: 168, 170-183, 189, 190 and 342. In some embodiments, at least one of the one or more peptides used in the methods of the disclosure is selected from any one of the sequences set forth in SEQ ID NO: 168, 170-183, 189, 190 and 342. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 168, 170-183, 189, 190 and 342.

In some embodiments, the one or more peptides used in the methods of the disclosure are selected from the group consisting of peptides being characterized by the amino acid sequences set forth in SEQ ID NO: 170-183, 189, 190, 341 and 342. In some embodiments, at least one of the one or more peptides used in the methods of the disclosure is selected from any one of the sequences set forth in SEQ ID NO: 170-183, 189, 190, 341 and 342. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 170-183, 189, 190, 341 and 342.

In some embodiments, the one, five, ten or more peptides used in the methods of the disclosure are selected from the group consisting of peptides being characterized by the amino acid sequences set forth in SEQ ID NO: 170, 172, 174, 175, 178, 179, 181, 190, 341, 342 and 345-350, and optionally one or more peptides being characterized by the amino acid sequences set forth in SEQ ID NO: 284-289. In some embodiments, at least one, at least five, at least ten of the one or more peptides used in the methods of the disclosure is selected from any one of the sequences set forth in SEQ ID NO: 170, 172, 174, 175, 178, 179, 181, 190, 341, 342 and 345-350, and optionally one or more peptides being characterized by the amino acid sequences set forth in SEQ ID NO: 284-289. In some embodiments, at least one, at least five, at least ten or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 170, 172, 174-175, 178, 179, 181, 190, 341, 342 and 345-350, and optionally one or more peptides being characterized by the amino acid sequences set forth in SEQ ID NO: 284-289.

In some embodiments, the one, five, ten, fifteen or more peptides used in the methods of the disclosure are selected from the group consisting of peptides being characterized by the amino acid sequences set forth in SEQ ID NO: 170, 172, 174, 175, 178, 179, 181, 190, 284-289, 341, 342 and 345-350. In some embodiments, at least one, at least five, at least ten, at least fifteen of the one or more peptides used in the methods of the disclosure is selected from any one of the sequences set forth in SEQ ID NO: 170, 172, 174, 175, 178, 179, 181, 190, 284-289, 341, 342 and 345-350. In some embodiments, at least one, at least five, at least ten, at least fifteen or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 170, 172, 174, 175, 178, 179, 181, 190, 284-289, 341, 342 and 345-350.

In some embodiments, the one or more peptides used in the methods of the disclosure are selected from the group consisting of peptides being characterized by the amino acid sequences set forth in SEQ ID NO: 168-172. In some embodiments, at least one of the one or more peptides used in the methods of the disclosure is selected from any one of the sequences set forth in SEQ ID NO: 168-172. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 168-172.

In some embodiments, the one or more peptides used in the methods of the disclosure are selected from the group consisting of peptides being characterized by the amino acid sequences set forth in SEQ ID NO: 168, 170-172 and 342. In some embodiments, at least one of the one or more peptides used in the methods of the disclosure is selected from any one of the sequences set forth in SEQ ID NO: 168, 170-172 and 342. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 168, 170-172 and 342.

In some embodiments, the one or more peptides used in the methods of the disclosure are selected from the group consisting of peptides being characterized by the amino acid sequences set forth in SEQ ID NO: 170-172, 341 and 342. In some embodiments, at least one of the one or more peptides used in the methods of the disclosure is selected from any one of the sequences set forth in SEQ ID NO: 170-172, 341 and 342. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 170-172, 341 and 342.

In some embodiments, the one or more peptides used in the methods of the disclosure are selected from the group consisting of peptides being characterized by the amino acid sequences set forth in SEQ ID NO: 170, 172, 341 and 342. In some embodiments, at least one of the one or more peptides used in the methods of the disclosure is selected from any one of the sequences set forth in SEQ ID NO: 170, 172, 341 and 342. In some embodiments, at least one or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 170, 172, 341 and 342.

In some embodiments, the one, five or more of the one or more peptides used in the methods of the disclosure are selected from the group consisting of peptides being characterized by the amino acid sequences set forth in SEQ ID NO: 173-183. In some embodiments, at least one, at least five of the one or more peptides used in the methods of the disclosure is selected from any one of the sequences set forth in SEQ ID NO: 173-183. In some embodiments, at least one, at least five or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 173-183.

In some embodiments, the one, five, ten or more peptides used in the methods of the disclosure are selected from the group consisting of peptides being characterized by the amino acid sequences set forth in SEQ ID NO: 173-183, 189 and 190. In some embodiments, at least one, at least five, at least ten of the one or more peptides used in the methods of the disclosure is selected from any one of the sequences set forth in SEQ ID NO: 173-183, 189 and 190. In some embodiments, at least one, at least five, at least ten or more of the one or more peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 173-183, 189 and 190.

In some embodiments, the one, five, ten or more peptides used in the methods of the disclosure are selected from the group consisting of peptides being characterized by the amino acid sequences set forth in SEQ ID NO: 174-176, 178, 179, 181, 190 and 345-350, and optionally one or more peptides being characterized by the amino acid sequences set forth in SEQ ID NO: 284-289. In some embodiments, at least one, at least five, at least ten of the one or more peptides used in the methods of the disclosure is selected from any one of the sequences set forth in SEQ ID NO: 174-176, 178, 179, 181, 190 and 345-350, and optionally one or more peptides being characterized by the amino acid sequences set forth in SEQ ID NO: 284-289. In some embodiments, at least one, at least five, at least ten or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 174-176, 178, 179, 181, 190 and 345-350, and optionally one or more peptides being characterized by the amino acid sequences set forth in SEQ ID NO: 284-289.

In some embodiments, the one, five, ten, fifteen or more peptides used in the methods of the disclosure are selected from the group consisting of peptides being characterized by the amino acid sequences set forth in SEQ ID NO: 174-176, 178, 179, 181, 190, 284-289 and 345-350. In some embodiments, at least one, at least five, at least ten, at least fifteen of the one or more peptides used in the methods of the disclosure is selected from any one of the sequences set forth in SEQ ID NO: 174-176, 178, 179, 181, 190, 284-289 and 345-350. In some embodiments, at least one, at least five, at least ten, at least fifteen or more of the one or more peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 174-176, 178, 179, 181, 190, 284-289 and 345-350.

In some embodiments, the one, five, ten, fifteen or more peptides used in the methods of the disclosure are selected from the group consisting of peptides being characterized by the amino acid sequences set forth in SEQ ID NO: 168, 170, 171, 173, 175, 176, 178, 179, 181, 190, and 342-350. In some embodiments, at least one, at least five, at least ten, at least fifteen of the one or more peptides used in the methods of the disclosure is selected from any one of the sequences set forth in SEQ ID NO: 168, 170, 171, 173, 175, 176, 178, 179, 181, 190, and 342-350. In some embodiments, at least one, at least five, at least ten, at least fifteen or more of the one or more peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 168, 170, 171, 173, 175, 176, 178, 179, 181, 190, and 342-350.

In some embodiments, the one, five, ten or more peptides used in the methods of the disclosure are selected from the group consisting of peptides being characterized by the amino acid sequences set forth in SEQ ID NO: 170, 175, 176, 178, 179, 181, 190, and 342-350, and optionally one or more peptides being characterized by the amino acid sequences set forth in SEQ ID NO: 168, 171 and 173. In some embodiments, at least one, at least five, at least ten of the one or more peptides used in the methods of the disclosure is selected from any one of the sequences set forth in SEQ ID NO: 170, 175, 176, 178, 179, 181, 190, and 342-350, and optionally one or more peptides being characterized by the amino acid sequences set forth in SEQ ID NO: 168, 171 and 173. In some embodiments, at least one, at least five, at least ten, at least fifteen or more of the one or more peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 170, 175, 176, 178, 179, 181, 190, and 342-350, and optionally one or more peptides being characterized by the amino acid sequences set forth in SEQ ID NO: 168, 171 and 173.

In some embodiments, the one or more peptides used in the methods of the disclosure are selected from the group consisting of peptides being characterized by the amino acid sequences set forth in SEQ ID NO: 168, 170, 171, 342 and 343. In some embodiments, at least one of the one or more peptides used in the methods of the disclosure is selected from any one of the sequences set forth in SEQ ID NO: 168, 170, 171, 342 and 343. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 168, 170, 171, 342 and 343.

In some embodiments, the one, five ten or more peptides used in the methods of the disclosure are selected from the group consisting of peptides being characterized by the amino acid sequences set forth in SEQ ID NO: 173, 175, 176, 178, 179, 181, 190 and 344-350. In some embodiments, at least one, at least five, at least ten of the one or more peptides used in the methods of the disclosure is selected from any one of the sequences set forth in SEQ ID NO: 173, 175, 176, 178, 179, 181, 190 and 344-350. In some embodiments, at least one, at least five, at least ten or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 173, 175, 176, 178, 179, 181, 190 and 344-350.

In some embodiments, the one or more peptides used in the methods of the disclosure are selected from the group consisting of peptides being characterized by the amino acid sequences set forth in SEQ ID NO: 191-193. In some embodiments, at least one of the one or more peptides used in the methods of the disclosure is selected from any one of the sequences set forth in SEQ ID NO: 191-193. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 191-193.

In some embodiments, the one, five, ten, fifteen, twenty, twenty-five, thirty or more peptides used in the methods of the disclosure are selected from the group consisting of peptides being characterized by the amino acid sequences set forth in SEQ ID NO: 235-248, 250-255, 257-261, 266, 268, 272, 274, 275, 277-283, 290, 291, 351-353 and 358. In some embodiments, at least one, at least five, at least ten, at least fifteen, at least twenty, at least twenty-five, at least thirty of the one or more peptides used in the methods of the disclosure is selected from any one of the sequences set forth in SEQ ID NO: 235-248, 250-255, 257-261, 266, 268, 272, 274, 275, 277-283, 290, 291, 351-353 and 358. In some embodiments, at least one, at least five, at least ten, at least fifteen, at least twenty, at least twenty-five, at least thirty or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 235-248, 250-255, 257-261, 266, 268, 272, 274, 275, 277-283, 290, 291, 351-353 and 358.

In some embodiments, the one, five, ten, fifteen, twenty, twenty-five, thirty or more peptides used in the methods of the disclosure are selected from the group consisting of peptides being characterized by the amino acid sequences set forth in SEQ ID NO: 235-248, 250, 252-255, 257-261, 268, 278-281, 290, 291, 351-353 and 358, and optionally one or more peptides comprising the amino acid sequences set forth in SEQ ID NO: 251, 266, 272, 274, 275, 277, 282 and 283. In some embodiments, at least one, at least five, at least ten, at least fifteen, at least twenty, at least twenty-five, at least thirty of the one or more peptides used in the methods of the disclosure is selected from any one of the sequences set forth in SEQ ID NO: 235-248, 250, 252-255, 257-261, 268, 278-281, 290, 291, 351-353 and 358, and optionally one or more peptides comprising the amino acid sequences set forth in SEQ ID NO: 251, 266, 272, 274, 275, 277, 282 and 283. In some embodiments, at least one, at least five, at least ten, at least fifteen, at least twenty, at least twenty-five, at least thirty or more of the one or more peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 235-248, 250, 252-255, 257-261, 268, 278-281, 290, 291, 351-353 and 358, and optionally one or more peptides comprising the amino acid sequences set forth in SEQ ID NO: 251, 266, 272, 274, 275, 277, 282 and 283.

In some embodiments, the one, five, ten, fifteen or more peptides used in the methods of the disclosure are selected from the group consisting of peptides being characterized by the amino acid sequences set forth in SEQ ID NO: 293, 295-298, 301, 302, 304-306, 313-316, 322, 328, 329. In some embodiments, at least one, at least five, at least ten, at least fifteen of the one or more peptides used in the methods of the disclosure is selected from any one of the sequences set forth in SEQ ID NO: 293, 295-298, 301, 302, 304-306, 313-316, 322, 328, 329. In some embodiments, at least one, at least five, at least ten, at least fifteen or more of the one or more peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 293, 295-298, 301, 302, 304-306, 313-316, 322, 328, 329.

In some embodiments, the one, five, ten, fifteen, twenty, twenty-five, thirty or more peptides used in the methods of the disclosure comprise one, five, ten, fifteen, twenty, twenty-five, thirty or more peptides selected from amino acid sequences set forth in SEQ ID NO: 1-45. In some embodiments, the one, five, ten, fifteen, twenty, twenty-five, thirty or more peptides used in the methods of the disclosure comprise one or more peptides selected from amino acid sequences set forth in SEQ ID NO: 46-167. In some embodiments, the one, five, ten or more peptides used in the methods of the disclosure comprise one or more peptides selected from amino acid sequences set forth in SEQ ID NO: 168-183, 189 and 190.

In some embodiments, the one, five, ten, fifteen, twenty, twenty-five, thirty or more peptides used in the methods of the disclosure comprise one or more peptides selected from amino acid sequences set forth in SEQ ID NO: 46-70, 72-94, 96, 97, 99-101, 103-107, 109-111, 113-149, 151-167, 307, 317, 319, 320, 323, 324 and 335. In some embodiments, the one, five, ten, fifteen, twenty, twenty-five, thirty or more peptides used in the methods of the disclosure comprise one, five, ten, fifteen, twenty, twenty-five, thirty or more peptides selected from amino acid sequences set forth in SEQ ID NO: 46-70, 72-94, 96, 97, 99-101, 103-107, 109-111, 113-149, and 151-167.

In some embodiments, the one, five, ten, fifteen, twenty, twenty-five or more peptides used in the methods of the disclosure are characterized by one or more peptides selected from amino acid sequences set forth in SEQ ID NO: 1-10, 12-25, 27, 28, 30-39, 41, 43 and 44. In some embodiments, the one, five, ten, fifteen, twenty, twenty-five, thirty or more peptides used in the methods of the disclosure are characterized by one or more peptides selected from amino acid sequences set forth in SEQ ID NO: 47, 50-55, 59, 60, 62-64, 66-70, 72, 74-77, 79-81, 83, 84, 86, 87, 90-94, 96, 97, 99-101, 103, 110, 114-126, 129, 131-134, 135-138, 140, 144-146, 148,149 153-167, 294, 299, 300, 303, 307, 308, 310, 311, 312, 317, 319, 320, 324, 325, 327, 333, 336 and 337, and optionally one or more peptides being characterized by the amino acid sequences set forth in SEQ ID NO: 235-282. In some embodiments, the one, five, ten, fifteen, twenty, twenty-five, thirty or more peptides used in the methods of the disclosure are characterized by one or more peptides selected from amino acid sequences set forth in SEQ ID NO: 47, 50-55, 59, 60, 62-64, 66-70, 72, 74-77, 79-81, 83, 84, 86, 87, 90-94, 996, 97, 99-101, 103, 110, 114-126, 129, 131-134, 135-138, 140, 144-146, 148,149, 153-167, 235-282, 294, 299, 300, 303, 307, 308, 310, 311, 312, 317, 319, 320, 324, 325, 327, 333, 336 and 337. In some embodiments, the one, five, ten or more peptides used in the methods of the disclosure are characterized by one or more peptides selected from amino acid sequences set forth in SEQ ID NO: 170, 172, 174-175, 178, 179, 181, 190, 341, 342 and 345-350, and optionally one or more peptides being characterized by the amino acid sequences set forth in SEQ ID NO: 284-289. In some embodiments, the one or more peptides used in the methods of the disclosure are characterized by one or more peptides selected from amino acid sequences set forth in SEQ ID NO: 170, 172, 174-175, 178, 179, 181, 190, 284-289, 341, 342 and 345-350.

In some embodiments, the one, five, ten, fifteen, twenty, twenty-five, thirty or more peptides used in the methods of the disclosure are characterized by one or more peptides selected from amino acid sequences set forth in SEQ ID NO: 1-10, 12-14, 17, 18, 20-25, 27, 28, 30-39, 41, 43, 44 and 292, and optionally one or more peptides being characterized by the amino acid sequences set forth in SEQ ID NO: 11, 16, 19, 26, 29, 40, 42, and 45. In some embodiments, the one, five, ten, fifteen, twenty, twenty-five, thirty or more peptides used in the methods of the disclosure are characterized by one or more peptides selected from amino acid sequences set forth in SEQ ID NO: 1-14, 16-45 and 292. In some embodiments, the one, five, ten, fifteen, twenty, twenty-five, thirty or more peptides used in the methods of the disclosure are characterized by one or more peptides selected from amino acid sequences set forth in SEQ ID NO: 47, 48, 50-55, 59, 60, 62-64, 66-70, 72, 74, 75, 77, 79-81, 83, 84, 86, 87, 90-94, 96, 99-101, 103, 110, 115-125, 129, 131-133, 136-138, 140, 142, 144, 146, 148,149, 153-155, 158, 159, 294, 299, 300, 303, 308, 309, 311, 312, 317-321, 324, 325, 330-334, 337-340, and 354-357, and optionally one or more peptides being characterized by the amino acid sequences set forth in SEQ ID NO: 46, 47, 58, 61, 71, 76, 89, 105-109, 111, 113, 114, 126, 128, 130, 139, 141, 147, 151, 152, 160-167, and 265. In some embodiments, the one, five, ten, fifteen, twenty, twenty-five, thirty or more peptides used in the methods of the disclosure are characterized by one or more peptides selected from amino acid sequences set forth in SEQ ID NO: 46-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-167, 265, 294, 299, 300, 303, 308, 309, 311, 312, 317-321, 324, 325, 330-334, 337-340, and 354-357. In some embodiments, the one, five, ten, fifteen or more peptides used in the methods of the disclosure are characterized by one or more peptides selected from amino acid sequences set forth in SEQ ID NO: 170, 175, 176, 178, 179, 181, 190, and 342-350, and optionally one or more peptides being characterized by the amino acid sequences set forth in SEQ ID NO: 168, 171, and 173. In some embodiments, the one, five, ten, fifteen or more peptides used in the methods of the disclosure are characterized by one or more peptides selected from amino acid sequences set forth in SEQ ID NO: 168, 170, 171, 173, 175, 176, 178, 179, 181, 190, and 342-350.

In some embodiments, the one, five, ten, fifteen, twenty, twenty-five, thirty or more peptides used in the methods of the disclosure are characterized by one or more peptides selected from amino acid sequences set forth in SEQ ID NO: 235-248, 250, 252-255, 257-261, 268, 278-281, 290, 291, 351-353 and 358, and optionally one or more peptides being characterized by the amino acid sequences set forth in SEQ ID NO: 251, 266, 272, 274, 275, 277, 282 and 283. In some embodiments, the one, five, ten, fifteen, twenty, twenty-five, thirty or more peptides used in the methods of the disclosure are characterized by one or more peptides selected from amino acid sequences set forth in SEQ ID NO: 235-248, 250-255, 257-261, 266, 268, 272, 274, 275, 277-283, 290, 291, 351-353 and 358. In some embodiments, the one, five, ten, fifteen, twenty, twenty-five, thirty or more peptides used in the methods of the disclosure are characterized by one or more peptides selected from amino acid sequences set forth in SEQ ID NO: 293, 295-298, 301, 302, 304-306, 313-316, 322, 328 and 329.

In some embodiments, the one or more peptides used in the methods of the disclosure comprise one or more peptides selected from amino acid sequences set forth in SEQ ID NO: 168, 170-183, 189, 190 and 342. In some embodiments, the one or more peptides used in the methods of the disclosure comprise one or more peptides selected from amino acid sequences set forth in SEQ ID NO: 170-183, 189, 190, 341 and 342. In some embodiments, the one or more peptides used in the methods of the disclosure comprise one or more peptides selected from amino acid sequences set forth in SEQ ID NO: 168, 170-183, 189 and 190. In some embodiments, the one or more peptides used in the methods of the disclosure comprise one or more peptides selected from amino acid sequences set forth in SEQ ID NO: 169, 170-183, 189 and 190. In some embodiments, the one or more peptides used in the methods of the disclosure comprise one or more peptides selected from amino acid sequences set forth in SEQ ID NO: 170-183, 189 and 190. In some embodiments, the one or more peptides used in the methods of the disclosure comprise one or more peptides selected from amino acid sequences set forth in SEQ ID NO: 168, 170, 171, 342 and 343. In some embodiments, the one or more peptides used in the methods of the disclosure comprise one or more peptides selected from amino acid sequences set forth in SEQ ID NO: 168, 170, 342 and 343. In some embodiments, the one, five, ten or more peptides used in the methods of the disclosure comprise one or more peptides selected from amino acid sequences set forth in SEQ ID NO: 173, 175, 176, 178, 179, 181, 190 and 344-350. In some embodiments, the one, five, ten or more peptides used in the methods of the disclosure comprise one or more peptides selected from amino acid sequences set forth in SEQ ID NO: 175, 176, 178, 179, 181, 190, and 344-350.

In some embodiments, the one or more peptides used in the methods of the disclosure are characterized by one or more peptides selected from amino acid sequences set forth in SEQ ID NO: 170, 172, 341 and 342. In some embodiments, the one or more peptides used in the methods of the disclosure are characterized by one or more peptides selected from amino acid sequences set forth in SEQ ID NO: 174-176, 178, 179, 181, 190 and 345-350, and optionally one or more peptides being characterized by the amino acid sequences set forth in SEQ ID NO: 284-289. In some embodiments, the one or more peptides used in the methods of the disclosure are characterized by one or more peptides selected from amino acid sequences set forth in SEQ ID NO: 174-176, 178, 179, 181, 190, 284-289 and 345-35-.

In some embodiments, the one, five, ten, fifteen, twenty, twenty-five or thirty or more peptides used in the methods of the disclosure comprise a combination of all the peptides being characterized by the amino acid sequences set forth in SEQ ID NO: 1-45. In some embodiments, the one or more peptides used in the methods of the disclosure comprise all the peptides of amino acid sequences set forth in SEQ ID NO: 1-45. In some embodiments, the one or more peptides used in the methods of the disclosure comprise at least a combination of peptides being characterized by the amino acid sequences set forth in SEQ ID NO: 1-45. In some embodiments, the one or more peptides used in the methods of the disclosure comprise at least all the peptides of amino acid sequences set forth in SEQ ID NO: 1-45. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 1-45.

In some embodiments, the one, five, ten, fifteen, twenty, twenty-five or more peptides used in the methods of the disclosure comprise a combination of all the peptides being characterized by the amino acid sequences set forth in SEQ ID NO: 1-10, 12-25, 27, 28, 30-39, 41, 43 and 44. In some embodiments, the one, five, ten, fifteen, twenty, twenty-five or more peptides used in the methods of the disclosure comprise all the peptides of amino acid sequences set forth in SEQ ID NO: 1-10, 12-25, 27, 28, 30-39, 41, 43 and 44. In some embodiments, the one, five, ten, fifteen, twenty, twenty-five or more peptides used in the methods of the disclosure comprise at least a combination of peptides being characterized by the amino acid sequences set forth in SEQ ID NO: 1-10, 12-25, 27, 28, 30-39, 41, 43 and 44. In some embodiments, the one, five, ten, fifteen, twenty, twenty-five or more peptides used in the methods of the disclosure comprise at least all the peptides of amino acid sequences set forth in SEQ ID NO: 1-10, 12-25, 27, 28, 30-39, 41, 43 and 44. In some embodiments, at least one, five, ten, fifteen, twenty, twenty-five or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 1-10, 12-25, 27, 28, 30-39, 41, 43 and 44.

In some embodiments, the one, five, ten, fifteen, twenty, twenty-five, thirty or more peptides used in the methods of the disclosure comprise a combination of all the peptides being characterized by the amino acid sequences set forth in SEQ ID NO: 46-167. In some embodiments, the one or more peptides used in the methods of the disclosure comprise all the peptides of amino acid sequences set forth in SEQ ID NO: 46-167. In some embodiments, the one, five, ten, fifteen, twenty, twenty-five, thirty or more peptides used in the methods of the disclosure comprise at least a combination of peptides being characterized by the amino acid sequences set forth in SEQ ID NO: 46-167. In some embodiments, the one, five, ten, fifteen, twenty, twenty-five, thirty or more peptides used in the methods of the disclosure comprise at least all the peptides of amino acid sequences set forth in SEQ ID NO: 46-167. In some embodiments, at least one, five, ten, fifteen, twenty, twenty-five, thirty or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 46-167.

In some embodiments, the one, five, ten, fifteen, twenty, twenty-five, thirty or more peptides used in the methods of the disclosure comprise at least a combination of peptides being characterized by the amino acid sequences set forth in SEQ ID NO: 46-70, 72-94, 96, 97, 99-101, 103-107, 109-111, 113-149, 151-167, 307, 317, 319, 320, 323, 324 and 335. In some embodiments, the one, five, ten, fifteen, twenty, twenty-five, thirty or more peptides used in the methods of the disclosure comprise at least all the peptides of amino acid sequences set forth in SEQ ID NO: 46-70, 72-94, 96, 97, 99-101, 103-107, 109-111, 113-149, 151-167, 307, 317, 319, 320, 323, 324 and 335. In some embodiments, at least one, five, ten, fifteen, twenty, twenty-five, thirty or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 46-70, 72-94, 96, 97, 99-101, 103-107, 109-111, 113-149, 151-167, 307, 317, 319, 320, 323, 324 and 335.

In some embodiments, the one, five, ten, fifteen, twenty, twenty-five, thirty or more peptides used in the methods of the disclosure comprise a combination of all the peptides being characterized by the amino acid sequences set forth in SEQ ID NO: 47, 50-55, 59, 60, 62-64, 66-70, 72, 74-77, 79-81, 83, 84, 86, 87, 90-94, 96, 97, 99-101, 103, 110, 114-126, 129, 131-134, 135-138, 140, 144-146, 148,149, 153-167, 294, 299, 300, 303, 307, 308, 310, 311, 312, 317, 319, 320, 324, 325, 327, 333, 336 and 337, and optionally one or more peptides being characterized by the amino acid sequences set forth in SEQ ID NO: 235-282. In some embodiments, the one, five, ten, fifteen, twenty, twenty-five, thirty or more peptides used in the methods of the disclosure comprise all the peptides of amino acid sequences set forth in SEQ ID NO: 47, 50-55, 59, 60, 62-64, 66-70, 72, 74-77, 79-81, 83, 84, 86, 87, 90-94, 96, 97, 99-101, 103, 110, 114-126, 129, 131-134, 135-138, 140, 144-146, 148, 149, 153-167, 294, 299, 300, 303, 307, 308, 310, 311, 312, 317, 319, 320, 324, 325, 327, 333, 336 and 337, and optionally one or more peptides being characterized by the amino acid sequences set forth in SEQ ID NO: 235-282. In some embodiments, the one, five, ten, fifteen, twenty, twenty-five, thirty or more peptides used in the methods of the disclosure comprise at least a combination of peptides being characterized by the amino acid sequences set forth in SEQ ID NO: 47, 50-55, 60, 62-64, 66-70, 72, 74-77, 79-81, 83, 84, 86, 87, 90-94, 96, 97, 99-101, 103, 110, 114-126, 129, 131-134, 135-138, 140, 144-146, 148, 149, 153-167, 294, 299, 300, 303, 307, 308, 310, 311, 312, 317, 319, 320, 324, 325, 327, 333, 336 and 337, and optionally one or more peptides being characterized by the amino acid sequences set forth in SEQ ID NO: 235-282. In some embodiments, the one, five, ten, fifteen, twenty, twenty-five, thirty or more peptides used in the methods of the disclosure comprise at least all the peptides of amino acid sequences set forth in SEQ ID NO: 47, 50-55, 59, 60, 62-64, 66-70, 72, 74-77, 79-81, 83, 84, 86, 87, 90-94, 96, 97, 99-101, 103, 110, 114-126, 129, 131-134, 135-138, 140, 144-146, 148,149, 153-167, 294, 299, 300, 303, 307, 308, 310, 311, 312, 317, 319, 320, 324, 325, 327, 333, 336 and 337, and optionally one or more peptides being characterized by the amino acid sequences set forth in SEQ ID NO: 235-282. In some embodiments, at least one, five, ten, fifteen, twenty, twenty-five, thirty or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 47, 50-55, 59, 60, 62-64, 66-70, 72, 74-77, 79-81, 83, 84, 86, 87, 90-94, 96, 97, 99-101, 103, 110, 114-126, 129, 131-134, 135-138, 140, 144-146, 148, 149, 153-167, 294, 299, 300, 303, 307, 308, 310, 311, 312, 317, 319, 320, 324, 325, 327, 333, 336 and 337, and optionally one or more peptides being characterized by the amino acid sequences set forth in SEQ ID NO: 235-282.

In some embodiments, the one, five, ten, fifteen, twenty, twenty-five, thirty or more peptides used in the methods of the disclosure comprise a combination of all the peptides being characterized by the amino acid sequences set forth in SEQ ID NO: 47, 50-55, 59, 60, 62-64, 66-70, 72, 74-77, 79-81, 83, 84, 86, 87, 90-94, 96, 97, 99-101, 103, 110, 114-126, 129, 131-134, 135-138, 140, 144-146, 148, 149, 153-167, and 235-282, 294, 299, 300, 303, 307, 308, 310, 311, 312, 317, 319, 320, 324, 325, 327, 333, 336 and 337. In some embodiments, the one, five, ten, fifteen, twenty, twenty-five, thirty or more peptides used in the methods of the disclosure comprise all the peptides of amino acid sequences set forth in SEQ ID NO: 47, 50-55, 59, 60, 62-64, 66-70, 72, 73a, 74-77, 79-81, 83, 84, 86, 87, 90-94, 96, 97, 99-101, 103, 110, 114-126, 129, 131-134, 135-138, 140, 144-146, 148,149, 153-167, 235-282, 294, 299, 300, 303, 307, 308, 310, 311, 312, 317, 319, 320, 324, 325, 327, 333, 336 and 337. In some embodiments, the one, five, ten, fifteen, twenty, twenty-five, thirty or more peptides used in the methods of the disclosure comprise at least a combination of peptides being characterized by the amino acid sequences set forth in SEQ ID NO: 47, 50-55, 60, 62-64, 66-70, 72, 74-77, 79-81, 83, 84, 86, 87, 90-94, 96, 97, 99-101, 103, 110, 114-126, 129, 131-134, 135-138, 140, 144-146, 148, 149, 153-167, 235-282, 294, 299, 300, 303, 307, 308, 310, 311, 312, 317, 319, 320, 324, 325, 327, 333, 336 and 337. In some embodiments, the one, five, ten, fifteen, twenty, twenty-five, thirty or more peptides used in the methods of the disclosure comprise at least all the peptides of amino acid sequences set forth in SEQ ID NO: 47, 50-55, 59, 60, 62-64, 66-70, 72, 74-77, 79-81, 83, 84, 86, 87, 90-94, 96, 97, 99-101, 103, 110, 114-126, 129, 131-134, 135-138, 140, 144-146, 148, 149, 153-167, 235-282, 294, 299, 300, 303, 307, 308, 310, 311, 312, 317, 319, 320, 324, 325, 327, 333, 336 and 337. In some embodiments, at least one, at least five, at least ten, at least fifteen, at least twenty, at least twenty-five, at least thirty or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 47, 50-55, 59, 60, 62-64, 66-70, 72, 74-77, 79-81, 83, 84, 86, 87, 90-94, 95a, 96, 97, 99-101, 103, 110, 114-126, 129, 131-134, 135-138, 140, 144-146, 148,149, 153-167, 235-282, 294, 299, 300, 303, 307, 308, 310, 311, 312, 317, 319, 320, 324, 325, 327, 333, 336 and 337.

In some embodiments, the one, five, ten or more peptides used in the methods of the disclosure comprise a combination of all the peptides being characterized by amino acid sequences set forth in SEQ ID NO: 168-183. In some embodiments, the one or more peptides used in the methods of the disclosure comprise a combination of all the peptides of amino acid sequences set forth in SEQ ID NO: 168-183. In some embodiments, the one, five, ten or more peptides used in the methods of the disclosure comprise at least a combination of peptides being characterized by amino acid sequences set forth in SEQ ID NO: 168-183. In some embodiments, the one, five, ten or more peptides used in the methods of the disclosure comprise at least all the peptides of amino acid sequences set forth in SEQ ID NO: 168-183. In some embodiments, at least one, five, ten or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 168-183.

In some embodiments, the one or more peptides used in the methods of the disclosure comprise at least a combination of peptides being characterized by amino acid sequences set forth in SEQ ID NO: 168-183, 189 and 190. In some embodiments, the one or more peptides used in the methods of the disclosure comprise at least all the peptides of amino acid sequences set forth in SEQ ID NO: 168-183, 189 and 190. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 168-183, 189 and 190.

In some embodiments, the one or more peptides used in the methods of the disclosure comprise at least a combination of peptides being characterized by amino acid sequences set forth in SEQ ID NO: 168, 170-183, 189, 190 and 342. In some embodiments, the one or more peptides used in the methods of the disclosure comprise at least all the peptides of amino acid sequences set forth in SEQ ID NO: 168, 170-183, 189, 190 and 342. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 168, 170-183, 189, 190 and 342.

In some embodiments, the one or more peptides used in the methods of the disclosure comprise at least a combination of peptides being characterized by amino acid sequences set forth in SEQ ID NO: 170-183, 189, 190, 341 and 342. In some embodiments, the one or more peptides used in the methods of the disclosure comprise at least all the peptides of amino acid sequences set forth in SEQ ID NO: 170-183, 189, 190, 341 and 342. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 170-183, 189, 190, 341 and 342.

In some embodiments, the one, five, tenor more peptides used in the methods of the disclosure comprise a combination of all the peptides being characterized by amino acid sequences set forth in SEQ ID NO: 170, 172, 174-175, 178, 179, 181, 190, 341, 342 and 345-350, and optionally one or more peptides being characterized by amino acid sequences set forth in SEQ ID NO: 284-289. In some embodiments, the one, five, ten or more peptides used in the methods of the disclosure comprise a combination of all the peptides of amino acid sequences set forth in SEQ ID NO: 170, 172, 174-175, 178, 179, 181, 190, 341, 342 and 345-350, and optionally one or more peptides being characterized by amino acid sequences set forth in SEQ ID NO: 284-289. In some embodiments, the one, five, ten or more peptides used in the methods of the disclosure comprise at least a combination of peptides being characterized by amino acid sequences set forth in SEQ ID NO: 170, 172, 174-175, 178, 179, 181, 190, 341, 342 and 345-350, and optionally one or more peptides being characterized by amino acid sequences set forth in SEQ ID NO: 284-289. In some embodiments, the one, five, ten or more peptides used in the methods of the disclosure comprise at least all the peptides of amino acid sequences set forth in SEQ ID NO: 170, 172, 174-175, 178, 179, 181, 190, 341, 342 and 345-350, and optionally one or more peptides being characterized by amino acid sequences set forth in SEQ ID NO: 284-289. In some embodiments, at least one, at least five, at least ten or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 170, 172, 174-175, 178, 179, 181, 190, 341, 342 and 345-350, and optionally one or more peptides being characterized by amino acid sequences set forth in SEQ ID NO: 284-289.

In some embodiments, the one, five, ten, fifteen or more peptides used in the methods of the disclosure comprise a combination of all the peptides being characterized by amino acid sequences set forth in SEQ ID NO: 170, 172, 174-175, 178, 179, 181, 190, 284-289, 341, 342 and 345-350. In some embodiments, the one, five, ten, fifteen or more peptides used in the methods of the disclosure comprise a combination of all the peptides of amino acid sequences set forth in SEQ ID NO: 170, 172, 174-175, 178, 179, 181, 190, 284-289, 341, 342 and 345-350. In some embodiments, the one, five, ten, fifteen or more peptides used in the methods of the disclosure comprise at least a combination of peptides being characterized by amino acid sequences set forth in SEQ ID NO: 170, 172, 174-175, 178, 179, 181, 190, 284-289, 341, 342 and 345-350. In some embodiments, the one, five, ten, fifteen or more peptides used in the methods of the disclosure comprise at least all the peptides of amino acid sequences set forth in SEQ ID NO: 170, 172, 174-175, 178, 179, 181, 190, 284-289, 341, 342 and 345-350. In some embodiments, at least one, at least five, at least ten, at least fifteen or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 170, 172, 174-175, 178, 179, 181, 190, 284-289, 341, 342 and 345-350.

In some embodiments, the one, five, ten, fifteen, twenty, twenty-five, thirty or more peptides used in the methods of the disclosure comprise at least a combination of peptides being characterized by the amino acid sequences set forth in SEQ ID NO: 194-231. In some embodiments, the one, five, ten, fifteen, twenty, twenty-five, thirty or more peptides used in the methods of the disclosure comprise at least all the peptides of amino acid sequences set forth in SEQ ID NO: 194-231. In some embodiments, at least one, at least five, at least ten, at least fifteen, at least twenty, at least twenty-five, at least thirty or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 194-231.

In some embodiments, the one or more peptides used in the methods of the disclosure comprise a combination of all the peptides being characterized by amino acid sequences set forth in SEQ ID NO: 168-172. In some embodiments, the one or more peptides used in the methods of the disclosure comprise a combination of all the peptides of amino acid sequences set forth in SEQ ID NO: 168-172. In some embodiments, the one or more peptides used in the methods of the disclosure comprise at least a combination of peptides being characterized by amino acid sequences set forth in SEQ ID NO: 168-172. In some embodiments, the one or more peptides used in the methods of the disclosure comprise at least all the peptides of amino acid sequences set forth in SEQ ID NO: 168-172. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 168-172.

In some embodiments, the one or more peptides used in the methods of the disclosure comprise at least a combination of peptides being characterized by amino acid sequences set forth in SEQ ID NO: 168, 170-172 and 342. In some embodiments, the one or more peptides used in the methods of the disclosure comprise at least all the peptides of amino acid sequences set forth in SEQ ID NO: 168, 170-172 and 342. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 168, 170-172 and 342.

In some embodiments, the one or more peptides used in the methods of the disclosure comprise at least a combination of all the peptides being characterized by amino acid sequences set forth in SEQ ID NO: 170-172, 341 and 342. In some embodiments, the one or more peptides used in the methods of the disclosure comprise at least all the peptides of amino acid sequences set forth in SEQ ID NO: 170-172, 341 and 342. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 170-172, 341 and 342.

In some embodiments, the one or more peptides used in the methods of the disclosure comprise at least a combination of all the peptides being characterized by amino acid sequences set forth in SEQ ID NO: 170, 172, 341 and 342. In some embodiments, the one or more peptides used in the methods of the disclosure comprise at least all the peptides of amino acid sequences set forth in SEQ ID NO: 170, 172, 341 and 342. In some embodiments, at least one or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 170, 172, 341 and 342.

In some embodiments, the one, five or more peptides used in the methods of the disclosure comprise a combination of all the peptides being characterized by amino acid sequences set forth in SEQ ID NO: 173-183. In some embodiments, the one, five or more peptides used in the methods of the disclosure comprise a combination of all the peptides of amino acid sequences set forth in SEQ ID NO: 173-183. In some embodiments, the one, five or more peptides used in the methods of the disclosure comprise at least a combination of peptides being characterized by amino acid sequences set forth in SEQ ID NO: 173-183. In some embodiments, the one, five or more peptides used in the methods of the disclosure comprise at least all the peptides of amino acid sequences set forth in SEQ ID NO: 173-183. In some embodiments, at least one, at least five or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 173-183.

In some embodiments, the one or more peptides used in the methods of the disclosure comprise at least a combination of peptides being characterized by amino acid sequences set forth in SEQ ID NO: 173-183, 189 and 190. In some embodiments, the one or more peptides used in the methods of the disclosure comprise at least all the peptides of amino acid sequences set forth in SEQ ID NO: 173-183, 189 and 190. In some embodiments, at least one or more of the peptides comprise an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 173-183, 189 and 190.

In some embodiments, the one, five, ten or more peptides used in the methods of the disclosure comprise a combination of all the peptides being characterized by amino acid sequences set forth in SEQ ID NO: 174-176, 178, 179, 181, 190 and 345-350, and optionally one or more peptides being characterized by amino acid sequences set forth in SEQ ID NO: 284-289. In some embodiments, the one, five, ten or more peptides used in the methods of the disclosure comprise a combination of all the peptides of amino acid sequences set forth in SEQ ID NO: 174-176, 178, 179, 181, 190 and 345-350, and optionally one or more peptides being characterized by amino acid sequences set forth in SEQ ID NO: 284-289. In some embodiments, the one, five, ten or more peptides used in the methods of the disclosure comprise at least a combination of peptides being characterized by amino acid sequences set forth in SEQ ID NO: 174-176, 178, 179, 181, 190 and 345-350, and optionally one or more peptides being characterized by amino acid sequences set forth in SEQ ID NO: 284-289. In some embodiments, the one, five, ten or more peptides used in the methods of the disclosure comprise at least all the peptides of amino acid sequences set forth in SEQ ID NO: 174-176, 178, 179, 181, 190 and 345-350, and optionally one or more peptides being characterized by amino acid sequences set forth in SEQ ID NO: 284-289. In some embodiments, at least one, five, ten or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 174-176, 178, 179, 181, 190 and 345-350, and optionally one or more peptides being characterized by amino acid sequences set forth in SEQ ID NO: 284-289.

In some embodiments, the one, five, ten, fifteen or more peptides used in the methods of the disclosure comprise a combination of all the peptides being characterized by amino acid sequences set forth in SEQ ID NO: 174-176, 178, 179, 181, 190, 284-289 and 345-350. In some embodiments, the one, five, ten, fifteen or more peptides used in the methods of the disclosure comprise a combination of all the peptides of amino acid sequences set forth in SEQ ID NO: 174-176, 178, 179, 181, 190, 284-289 and 345-350. In some embodiments, the one, five, ten, fifteen or more peptides used in the methods of the disclosure comprise at least a combination of peptides being characterized by amino acid sequences set forth in SEQ ID NO: 174-176, 178, 179, 181, 190, 284-289 and 345-350. In some embodiments, the one, five, ten, fifteen or more peptides used in the methods of the disclosure comprise at least all the peptides of amino acid sequences set forth in SEQ ID NO: 174-176, 178, 179, 181, 190, 284-289 and 345-350. In some embodiments, at least one, at least five, at least ten, at least fifteen or more of the peptides are characterized by an amino acid sequence with at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequences of the corresponding peptides set forth in SEQ ID NO: 174-176, 178, 179, 181, 190, 284-289 and 345-350.

In some embodiments this disclosure, the one or more peptides comprise combinations of one or more of the groups of Paragraphs [0108]-[0185].

In some embodiments, the one, five, ten, fifteen, twenty, twenty-five, thirty or more peptides used in the methods of the disclosure are peptide variants comprising at least one amino acid substitution, deletion, or insertion relative to the corresponding amino acid sequence of any one of SEQ ID NO: 1-183. In some embodiments, the one, five, ten, fifteen, twenty, twenty-five, thirty or more peptides used in the methods of the disclosure are peptide variants comprising at least one amino acid substitution, deletion, or insertion relative to the corresponding amino acid sequence of any one of SEQ ID NO: 1-183 and 189-231. In some embodiments, the one, five, ten, fifteen, twenty, twenty-five, thirty or more peptides used in the methods of the disclosure are peptide variants comprising at least one amino acid substitution, deletion, or insertion relative to the corresponding amino acid sequence of any one of SEQ ID NO: 1-358. In some embodiments, the one, five, ten, fifteen, twenty, twenty-five, thirty or more peptides used in the methods of the disclosure are peptide variants comprising at least one amino acid substitution, deletion, or insertion relative to the corresponding amino acid sequence of any one of SEQ ID NO: 1-183, 189-231 and 234-358. In some embodiments, the one, five, ten, fifteen, twenty, twenty-five, thirty or more peptides used in the methods of the disclosure are peptide variants comprising at least one amino acid substitution, deletion, or insertion relative to the corresponding amino acid sequence of any one of SEQ ID NO: 1-183, 189-232, 294, 299, 300, 303, 307-312, 317, 319, 320, 324-327, 333 and 335-337, 341 and 342. In some embodiments, the one, five, ten, fifteen, twenty, twenty-five, thirty or more peptides used in the methods of the disclosure are peptide variants being characterized by at least one amino acid substitution, deletion, or insertion relative to the corresponding amino acid sequence of any one of SEQ ID NO: 1-104, 109-138, 140-183, 189-231, 235-289, 294, 299, 303, 307-312, 317, 319, 320, 324-327, 333, 335-337, 341, 342 and 345-350. In some embodiments, the one, five, ten, fifteen, twenty, twenty-five, thirty or more peptides used in the methods of the disclosure are peptide variants being characterized by at least one amino acid substitution, deletion, or insertion relative to the corresponding amino acid sequence of any one of SEQ ID NO: 1-14, 16-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-168, 170, 171, 173, 175, 176, 178, 179, 181, 190, 235-248, 250-255, 257-261, 266, 268, 265, 272, 274, 275, 277-283, 290-306, 308-322, 324, 325, 328, 328, 330-334, 337-340, and 342-358. In some embodiments, variants are synthetic, recombinant, or chemically modified peptides isolated or generated using methods well known in the art. In some embodiments, variants include conservative or non-conservative amino acid changes, as described below. Polynucleotide changes can result in amino acid substitutions, additions, deletions, fusions and truncations in the polypeptide encoded by the reference sequence. In some embodiments, variants include insertions, deletions or substitutions of amino acids, including insertions and substitutions of amino acids and other molecules that do not normally occur in the peptide sequence that is the basis of the variant, for example but not limited to insertion of ornithine which do not normally occur in human proteins. The term conservative substitution, when describing a peptide, refers to a change in the amino acid composition of the peptide that does not substantially alter the peptide's activity. For example, a conservative substitution refers to substituting an amino acid residue for a different amino acid residue that has similar chemical properties. Examples of conservative amino acid substitutions include, but are not limited to, replacement of a leucine with an isoleucine or valine, an aspartate with a glutamate, or a threonine with a serine. In some embodiments, a conservative substitution of a particular amino acid sequence refers to substitution of those amino acids that are not critical for polypeptide activity or substitution of amino acids with other amino acids having similar properties (e.g., acidic, basic, positively or negatively charged, polar or non-polar) such that the substitution of even critical amino acids does not reduce the activity of the peptide. Conservative substitution providing functionally similar amino acids are well-known in the art. For example, the following six groups each contain amino acids that are conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W). (See, also Creighton, Proteins, W. H. Freeman and Company (1984), incorporated by reference herein in its entirety). In some embodiments, for ease of synthesis, one or more cysteines in a peptide of this disclosure may be replaced with serine. In some embodiments, for ease of synthesis, one or more peptides of this disclosure may be truncated. In some embodiments, for ease of synthesis, one or more peptides of this disclosure may be synthesized and used in fragments or recombined into single peptide.

Methods to produce the peptides of the disclosure are well-known in the art. See, e.g., Lloyd-Williams P. et al. (1997) Chemical approaches to the synthesis of peptides and proteins. CRC Press. Boca Raton and N. Leo Benoiton (2006) Chemistry of Peptide Synthesis. CRC Press. Boca Raton; incorporated by reference herein in their entireties.

In some embodiments, the one or more peptides of the disclosure are manufactured through solid-phase peptide synthesis (SPPS). In some embodiments, the solid support, for example, consists of small, polymeric resin beads functionalized with reactive groups (such as amine or hydroxyl groups) that link to the nascent peptide chain. In some embodiments, the protection of the N-terminal and side chains is performed using the Boc/Bzl or Fmoc/tBu SPPS approaches.

In some embodiments, the one or more peptides of the disclosure are manufactured as Trifluoroacetate (TFA) salts. In some embodiments, the residual TFA present in the peptides is removed before using any of the peptides in the methods of the disclosure.

Methods to purify peptides are well-known in the art. See, e.g., Insuasty Cepeda D S et al. Molecules. 2019; 24(7):1215; incorporated by reference herein in its entirety. In some embodiments, the peptides of the disclosure are purified through Ultra Performance Liquid Chromatography (UPLC). A skilled in the art will know methods to identify and to determine the purity of the manufactured peptides. In some embodiments, the one or more peptides of the disclosure are substantially pure.

The sequences referenced herein are provided in Table 1, infra.

TABLE 1
Compilation of amino acid sequences described in the present disclosure.
SARS-CoV-2 PEPTIDE SEQUENCES

| | | POSITION | | | POSITION |
|---|---|---|---|---|---|
| | SEQ | IN THE | | SEQ | IN THE |
| | ID | S | | ID | S |
| PEPTIDE SEQUENCE | NO: | PROTEIN | PEPTIDE SEQUENCE | NO: | PROTEIN |
| IRGWIFGTTLDSKTQSLL | 1 | 101-118 | NGVGYQPYRVVVLSFELLHA | 96 | 501-520 |
| CTFEYVSQPFLMD | 2 | 166-178 | VVLSFELLHAPATVCGPKKS | 97 | 511-530 |
| QPFLMDLEGKQGN | 3 | 173-185 | PATVCGPKKSTNLVKNKCV | 98 | 521-540 |
| | | | N | | |
| TRFQTLLALHRSYLTPGDSSS | 4 | 236-258 | TNLVKNKCVNFNFNGLTGT | 99 | 531-550 |
| GW | | | G | | |
| KSFTVEKGIYQTSNFRVQ | 5 | 304-321 | FNFNGLTGTGVLTESNKKFL | 100 | 541-560 |
| SASFSTFKCYGVSPTKL | 6 | 371-387 | VLTESNKKFLPFQQFGRDIA | 101 | 551-570 |
| KLPDDFTGCV | 7 | 424-433 | PFQQFGRDIADTTDAVRDPQ | 102 | 561-580 |
| NLDSKVGGNYNYLYRLFR | 8 | 440-457 | DTTDAVRDPQTLEILDITP | 103 | 571-589 |
| YLYRLFRKSNLKPFERDI | 9 | 451-468 | TLEILDITPCSFGGVSVITP | 104 | 581-600 |
| KPFERDISIEIYQ | 10 | 462-474 | SFGGVSVITPECDIPIGAGI | 105 | 591-600 |
| | | | | | and |
| | | | | | 661-670 |
| QSIIAYTMSLGAENSVAY | 11 | 690-707 | ECDIPIGAGICASYQTQTNS | 106 | 661-680 |
| SIIAYTMSL | 12 | 691-699 | CASYQTQTNSPRRARSVASQ | 107 | 671-690 |
| TECSNLLLQYGSFCTQL | 13 | 747-763 | PRRARSVASQSIIAYTMSLG | 108 | 681-700 |
| VKQIYKTPPIKDFGGFNF | 14 | 785-802 | SIIAYTMSLGAENSVAYSNN | 109 | 691-710 |
| DSLSSTASALGKLQDVV | 15 | 936-952 | AENSVAYSNNSIAIPTNFTI | 110 | 701-720 |
| ALNTLVKQL | 16 | 958-966 | SIAIPTNFTISVTTEILPVS | 111 | 711-730 |
| VLNDILSRL | 17 | 976-984 | SVTTEILPVSMTKTSVDCTM | 112 | 721-740 |
| LITGRLQSL | 18 | 996-1004 | MTKTSVDCTMYICGDSTECS | 113 | 731-750 |
| QLIRAAEIRASANLAATK | 19 | 1011-1028 | YICGDSTECSNLLLQYGSF | 114 | 741-759 |
| HWFVTQRNFYEPQII | 20 | 1101-1115 | NLLLQYGSFCTQLNRALTGI | 115 | 751-770 |
| RLNEVAKNL | 21 | 1185-1193 | TQLNRALTGIAVEQDKNTQE | 116 | 761-780 |
| NLNESLIDL | 22 | 1192-1200 | AVEQDKNTQEVFAQVKQIY | 117 | 771-790 |
| | | | K | | |
| FIAGLIAIV | 23 | 1220-1228 | VFAQVKQIYKTPPIKDFGGF | 118 | 781-800 |
| MFVFLVLLPLVSSQCVNLTT | 46 | 1-20 | TPPIKDFGGFNFSQILPDPS | 119 | 791-810 |
| VSSQCVNLTTRTQLPPAYTN | 47 | 11-30 | NFSQILPDPSKPSKRSFIED | 120 | 801-820 |
| RTQLPPAYTNSFTRGVYYPD | 48 | 21-40 | KPSKRSFIEDLLFNKVTLAD | 121 | 811-830 |
| SFTRGVYYPDKVFRSSVLHS | 49 | 31-50 | LLFNKVTLADAGFIKQYGD | 122 | 821-839 |
| KVFRSSVLHSTQDLFLPFFS | 50 | 41-60 | AGFIKQYGDCLGDIAARDLI | 123 | 831-850 |
| TQDLFLPFFSNVTWFHAIHV | 51 | 51-70 | LGDIAARDLICAQKFNGLTV | 124 | 841-860 |
| NVTWFHAIHVSGTNGTKRFD | 52 | 61-80 | CAQKFNGLTVLPPLLTDEMI | 125 | 851-870 |
| SGTNGTKRFDNPVLPFNDGV | 53 | 71-90 | LPPLLTDEMIAQYTSALLAG | 126 | 861-880 |
| NPVLPFNDGVYFASTEKSNI | 54 | 81-100 | AQYTSALLAGTITSGWTFGA | 127 | 871-890 |
| YFASTEKSNIIRGWIFGTTL | 55 | 91-110 | TITSGWTFGAGAALQIPFAM | 128 | 881-900 |
| IRGWIFGTTLDSKTQSLLIV | 56 | 101-120 | GAALQIPFAMQMAYRFNGIG | 129 | 891-910 |
| DSKTQSLLIVNNATNVVIKV | 57 | 111-130 | QMAYRFNGIGVTQNVLYEN | 130 | 901-920 |
| | | | Q | | |
| NNATNVVIKVCEFQFCNDPF | 58 | 121-140 | VTQNVLYENQKLIANQFNSA | 131 | 911-930 |
| CEFQFCNDPFLGVYYHKNNK | 59 | 131-150 | KLIANQFNSAIGKIQDSLSS | 132 | 921-940 |
| LGVYYHKNNKSWMESEFRV | 60 | 141-160 | IGKIQDSLSSTASALGKLQD | 133 | 931-950 |
| Y | | | | | |
| SWMESEFRVYSSANNCTFEY | 61 | 151-170 | TASALGKLQDVVNQNAQAL | 134 | 941-960 |
| | | | N | | |
| SSANNCTFEYVSQPFLMDLE | 62 | 161-180 | VVNQNAQALNTLVKQLSSN | 135 | 951-970 |
| | | | F | | |
| VSQPFLMDLEGKQGNFKNLR | 63 | 171-190 | TLVKQLSSNFGAISSVLNDI | 136 | 961-980 |
| GKQGNFKNLREFVFKNIDGY | 64 | 181-200 | GAISSVLNDILSRLDKVEAE | 137 | 971-990 |
| EFVFKNIDGYFKIYSKHTPI | 65 | 191-210 | LSRLDKVEAEVQIDRLITGR | 138 | 981-1000 |
| FKIYSKHTPINLVRDLPQGF | 66 | 201-220 | VQIDRLITGRLQSLQTYVTQ | 139 | 991-1010 |
| NLVRDLPQGFSALEPLVDLP | 67 | 211-230 | LQSLQTYVTQQLIRAAEIRA | 140 | 1001-1020 |
| SALEPLVDLPIGINITRFQT | 68 | 221-240 | QLIRAAEIRASANLAATKMS | 141 | 1011-1030 |
| IGINITRFQTLLALHRSYLT | 69 | 231-250 | SANLAATKMSECVLGQSKR | 142 | 1021-1040 |
| | | | V | | |
| LLALHRSYLTPGDSSSGWTA | 70 | 241-260 | ECVLGQSKRVDFCGKGYHL | 143 | 1031-1050 |
| | | | M | | |
| PGDSSSGWTAGAAAYYVGYL | 71 | 251-270 | DFCGKGYHLMSFPQSAPHGV | 144 | 1041-1060 |
| GAAAYYVGYLQPRTFLLKYN | 72 | 261-280 | SFPQSAPHGVVFLHVTYVPA | 145 | 1051-1070 |
| QPRTFLLKYNENGTITDAVD | 73 | 271-290 | VFLHVTYVPAQEKNFTTAPA | 146 | 1061-1080 |
| ENGTITDAVDCALDPLSETK | 74 | 281-300 | QEKNFTTAPAICHDGKAHFP | 147 | 1071-1090 |
| CALDPL ETKCTLKSFTVEK | 75 | 291-310 | ICHDGKAHFPREGVFVSNGT | 148 | 1081-1100 |
| CTLKSFTVEKGIYQTSNFRV | 76 | 301-320 | REGVFVSNGTHWFVTQRNF | 149 | 1091-1110 |
| | | | Y | | |
| GIYQTSNFRVQPTESIVRFP | 77 | 311-330 | HWFVTQRNFYEPQIITTDNT | 150 | 1101-1120 |
| QPTESIVRFPNITNLCPFGE | 78 | 321-340 | EPQIITTDNTFVSGNCDVVI | 151 | 1111-1130 |
| NITNLCPFGEVFNATRFASV | 79 | 331-350 | FVSGNCDVVIGIVNNTVYDP | 152 | 1121-1140 |
| VFNATRFASVYAWNRKRISN | 80 | 341-360 | GIVNNTVYDPLQPELDSFKE | 153 | 1131-1150 |
| YAWNRKRISNCVADYSVLYN | 81 | 351-370 | LQPELDSFKEELDKYFKNHT | 154 | 1141-1160 |
| CVADYSVLYNSASFSTFKCY | 82 | 361-380 | ELDKYFKNHTSPDVDLGDIS | 155 | 1151-1170 |
| SASFSTFKCYGVSPTKLNDL | 83 | 371-390 | SPDVDLGDISGINASVVNIQ | 156 | 1161-1180 |
| GVSPTKLNDLCFTNVYADSF | 84 | 381-400 | GINASVVNIQKEIDRLNEVA | 157 | 1171-1190 |
| CFTNVYADSFVIRGDEVRQI | 85 | 391-410 | KEIDRLNEVAKNLNESLIDL | 158 | 1181-1200 |
| VIRGDEVRQIAPGQTGKIAD | 86 | 401-420 | KNLNESLIDLQELGKYEQYI | 159 | 1191-1210 |
| APGQTGKIADYNYKLPDDFT | 87 | 411-430 | QELGKYEQYIKWPWYIWLG | 160 | 1201-1220 |
| | | | F | | |
| YNYKLPDDFTGCVIAWNSNN | 88 | 421-440 | KWPWYIWLGFIAGLIAIVMV | 161 | 1211-1230 |
| GCVIAWNSNNLDSKVGGNYN | 89 | 431-450 | IAGLIAIVMVTIMLCCMTS | 162 | 1221-1239 |
| LDSKVGGNYNYLYRLFRKSN | 90 | 441-460 | TIMLCCMTSCCSCLKGCCS | 163 | 1231-1249 |
| YLYRLFRKSNLKPFERDIST | 91 | 451-470 | CSCLKGCCSCGSCCKFDEDD | 164 | 1241-1260 |
| LKPFERDISILIYQAGSTP | 92 | 461-479 | GSCCKFDEDDSEPVLKGVKL | 165 | 1251-1273 |
| | | | HYT | | |
| EIYQAGSTPCNGVEGFNCYF | 93 | 471-490 | SNQVAVLYQGVNCIENPVA | 166 | 605-623 |
| | | | | | (D614G) |
| NGVEGFNCYFPLQSYGFQPT | 94 | 481-500 | IQDSLSSTAPALGKLQDVV | 167 | 934-952 |
| | | | | | (S943P) |
| PLQSYGFQPTNGVGYQPYRV | 95 | 491-510 | ATVCGPKKSTNLVKNKCVN | 98a | 522-540 |
| SFTRGVYYPDKVFRSSVL | 294 | 31-48 | FQQFGRDIADTTDAVRDPQ | 320 | 562-580 |
| IFGTTLDSK | 299 | 105-113 | RRARSVASQSIIAYTMSLG | 323 | 682-700 |
| DSKTQSLLIV | 300 | 111-120 | SVTTEILPVSMTKTSVDCT | 324 | 721-739 |
| FVFKNIDGYFKIYSKHTPI | 303 | 192-210 | ALLAGTITSG | 325 | 876-885 |
| GDSSSGWTAGAAAYYVGYL | 307 | 252-270 | TITSGWTFGAGAALQIPFA | 326 | 881-899 |
| RTFLLKYNENGTITDAV | 308 | 273-289 | TASALGKLQDVVNQNAQAL | 327 | 941-959 |
| PIESIVRFPNITNLCPFG | 309 | 322-339 | WFVTQRNFYEPQIITTDNT | 335 | 1102-1120 |
| VADYSVLYNSASFSTFK | 310 | 362-378 | TQRNFYEPQI | 336 | 1105-1114 |
| FTNVYADSFVIRGDEVRQI | 311 | 392-410 | PQIITTDNT | 337 | 1112-1120 |
| YNYKLPDDFTGCVIAWNS | 312 | 421-438 | TQRNFVEPQI | 150d | 1105-1114 |
| | | | | | (Y1110V) |
| LQSYGFQPTNGVGYQPYRV | 317 | 492-510 | ECVLGQSKRVDFCGKGYHL | 333 | 1031-1049 |
| VSSQCVNFTTRTQLPPAYTN | 235 | 11-30 | VLTESNKKFLPFQQFGRDID | 260 | 551-570 |
| | | (L18F) | | | (A570D) |
| VSSQCVNFTNRTQLPSAYTN | 236 | 11-30 | PFQQFGRDIDDTTDAVRDPQ | 261 | 561-580 |
| | | (L18F/T20N/ | | | (A570D) |
| | | P26S) | | | |
| RTQLPSAYTNSFTRGVYYPD | 237 | 21-40 | SFGGVSVITPGTNTSNQVAV | 262 | 591-610 |
| | | (P26S) | | | |
| TQDLFLPFFSNVTWFHAI | 238 | 51-68 | ITPGTNTSNQVAVLYQDVNC | 263 | 598-617 |
| NVTWFHAISGTNGTKRFD | 239 | 61-68 & | ITPGTNTSNQVAVLYQGVNC | 264 | 598-617 |
| | | 71-78 | | | (D614G) |
| NVTWFHAIHVSGTNGTKRFA | 240 | 61-81 | GTNTSNQVAVLYQDVNCTE | 265 | 601-620 |
| | | (D80A) | V | | |
| SGTNGTKRFANPVLPFNDGV | 241 | 71-90 | GTNTSNQVAVLYQGVNCTE | 266 | 601-620 |
| | | (D80A) | V | | (D614G) |
| CEFQFCNYPFLGVYYHKNNK | 242 | 131-150 | LYQDVNCTEVPVAIHADQLT | 267 | 611-630 |
| | | (D138Y) | | | |
| CEFQFCNDPFLGVYHKNNK | 243 | 131-143 & | LYQGVNCIENPVAIHADQLT | 268 | 611-630 |
| | | 145-150 | | | (D614G) |
| | | (ΔY144) | | | |
| LGVYHKNNKSWMESEFRVY | 244 | 141-160 | PVAIHADQLTPTWRVYSTGS | 269 | 621-640 |
| | | (ΔY145) | | | |
| VSQPFLMDLEGKQGNFKNLS | 245 | 171-190 | PTWRVYSTGSNVFQTRAGCL | 270 | 631-650 |
| | | (R190S) | | | |
| GKQGNFKNLSEFVFKNIDGY | 246 | 181-200 | NVFQTRAGCLIGAEHVNNSY | 271 | 641-660 |
| | | (R190S) | | | |
| FKIYSKHTPINLVRGLPQGF | 247 | 201-220 | NVFQTRAGCLIGAEYVNNSY | 272 | 641-660 |
| | | (D215G) | | | (H655Y) |
| NLVRGLPQGFSALEPLVDLP | 248 | 211-230 | IGAEHVNNSYECDIPIGAGI | 273 | 651-670 |
| | | (D215G) | | | |
| IGINITRFQTLHISYLT | 249 | 231-240 & | IGAEYVNNSYECDIPIGAGI | 274 | 651-670 |
| | | 244-250 | | | (H655Y) |
| | | (R246I) | | | |
| LHISYLTPGDSSSGWTA | 250 | 244-260 | SIIAYTMSLGVENSVAYSNN | 275 | 691-710 |
| | | (R246I) | | | (Δ701V) |
| VIRGDEVRQIAPGQTGNIAD | 251 | 401-420 | VENSVAYSNNSIAIPTNFTI | 276 | 701-720 |
| | | (K417N) | | | (Δ701V) |
| VIRGDEVRQIAPGQTGTIAD | 252 | 401-420 | AENSVAYSNNSIAIPINFTI | 277 | 701-720 |
| | | (K417T) | | | (T716I) |
| APGQTGNIADYNYKLPDDFT | 253 | 411-430 | GAISSVLNDILARLDKVEAE | 278 | 971-990 |
| | | (K417N) | | | (S982A) |
| APGQTGTIADYNYKLPDDFT | 254 | 411-430 | LARLDKVEAEVQIDRLITGR | 279 | 981-1000 |
| | | (K417T) | | | (S982A) |
| EIYQAGSTPCNGVKGFNCYF | 255 | 471-490 | SANLAAIKMSECVLGQSKRV | 280 | 1021-1040 |
| | | (E484K) | | | (T1027I) |
| NGVKGFNCYFPLQSYGFQPT | 256 | 481-500 | HWFVTQRNFYEPQIITTHNT | 281 | 1101-1120 |
| | | (E484K) | | | (D1118H) |
| GFNCYFPLQSYGFQPTYGVG | 257 | 485-504 | PQIITTHNT | 282 | 1112-1120 |
| | | (N501Y) | | | (D1118H) |
| PLQSYGFQPTYGVGYQPYRV | 258 | 491-510 | EPQIITTHNTFVSGNCDVVI | 283 | 1111-1130 |
| | | (N501Y) | | | (D1118H) |
| YGVGYQPYRVVVLSFELLHA | 259 | 501-520 | VSSQCVNLRTRTQLPPAYTN | 293 | 11-30 |
| | | (N501Y) | | | (T19R) |
| TQDLFLPFFSNVTWFHAIHF | 295 | 51-70 | LLALHRSYLTPGDSSSGLTA | 306 | 241-260 |
| | | (V70F) | | | (W258L) |
| NVTWFHAIHFSGTNGTKRFD | 296 | 61-80 | LDSKVGGNYNYRYRLFRKS | 313 | 441-460 |
| | | (V70F) | N | | (L452R) |
| NPVLPFNDGVYFASIEKSNI | 299 | 81-100 | YRYRLFRKSNLKPFERDIST | 314 | 451-470 |
| | | (T95I) | | | (L452R) |
| YFASIEKSNIIRGWIFGTTL | 298 | 91-110 | LKPFERDISILIYQAGSKP | 315 | 461-479 |
| | | (T95I) | | | (T478K) |
| CEFQFCNDPFLDVYYHKNNK | 301 | 131-150 | EIYQAGSKPCNGVEGFNCYF | 316 | 471-490 |
| | | (G142D) | | | (T478K) |
| LDVYYHKNNKSWMESGVY | 301 | 141-155 | CASYQTQTNSRRRARSVASQ | 322 | 671-690 |
| | | and | | | (P681R) |
| | | 158-160 | | | |
| | | (G142D/ | | | |
| | | Δ156-157/ | | | |
| | | R158G) | | | |
| NLVRDLPQGFSVLEPLVDLP | 304 | 211-230 | TASALGKLQNVVNQNAQAL | 328 | 941-960 |
| | | (Δ222V) | N | | (D950N) |
| SVLEPLVDLPIGINITRFQT | 305 | 221-240 | TASALGKLQNVVNQNAQAL | 329 | 941-959 |
| | | (Δ222V) | | | (D950N) |
| DSLSSTASALGKLQDW | 292 | 936-952 | WLSFELLHAPATVCGPKKS | 318 | 512-530 |
| | | (V951D/ | | | (V512W) |
| | | V952W) | | | |
| TLEILDITPSSFGGVSVI | 321 | 581-598 | TASALGKLQDWNQNAQALN | 330 | 941-960 |
| | | (C590S) | | | (V951W/ |
| | | | | | ΔV952) |
| WNQNAQALNTLVKQLSSNF | 332 | 952-970 | TASALGKLQDWNQNAQAL | 331 | 941-959 |
| | | (V952W) | | | (V951W/ |
| | | | | | ΔV952) |
| SFPQSAPHGWFLHVTYVPA | 334 | 1051-1070 | LYQDVNSTEVPVAIHADQL | 354 | 611-629 |
| | | (V1060W/ | | | (C617S) |
| | | ΔV1061) | | | |
| SPDVDLGDISGINASWNIQ | 339 | 1161-1180 | VAIHADQLTPTWRVYSTGS | 355 | 622-640 |
| | | (V1176W/ | | | |
| | | ΔV1177) | | | |
| GINASWNIQKEIDRLNEVA | 340 | 1171-1190 | TWRVYSTGSNVFQTRAGSL | 356 | 632-650 |
| | | (V1176W/ | | | (C649S) |
| | | ΔV1177) | | | |
| RFQTLHISYLTPGDSS | 351 | 237-240 & | NVFQTRAGSLIGAEHVNNSY | 357 | 641-660 |
| | | 244-255 | | | (C649S) |
| | | (R246I) | | | |
| GVKGFNSYFPLQSYGF | 352 | 482-497 | VSIQCVNLTTRTQLPPAYTN | 290 | 11-30 |
| | | (E484K/ | | | (S13I) |
| | | C488S) | | | |
| GTNTSNQVAVLYQGV | 353 | 601-615 | LGVYYHKNNKSCMESEFRV | 291 | 141-160 |
| | | (D614G) | Y | | (W152C) |
| VENSVAYSNNSIAI | 358 | 701-714 | | | |

| | SEQ | |
|---|---|---|
| PROTEIN SEQUENCE | ID NO: | PROTEIN |
| MFVFLVLLPL VSSQCVNLTT RTQLPPAYTN SFTRGVYYPD | 184 | Full-length S |
| KVFRSSVLHS TQDLFLPFFS | | protein (1-1273) |
| NVTWFHAIHV SGTNGTKRFD NPVLPFNDGV YFASTEKSNI | | |
| IRGWIFGTTL DSKTQSLLIV | | |
| NNATNVVIKV CEFQFCNDPF LGVYYHKNNK SWMESEFRVY | | |
| SSANNCTFEY VSQPFLMDLE | | |
| GKQGNFKNLR EFVFKNIDGY FKIYSKHTPI NLVRDLPQGF | | |
| SALEPLVDLP IGINITRFQT | | |
| LLALHRSYLT PGDSSSGWTA GAAAYYVGYL QPRTFLLKYN | | |
| ENGTITDAVD CALDPLSETK | | |
| CTLKSFTVEK GIYQTSNFRV QPTESIVRFP NITNLCPFGE | | |
| VFNATRFASV YAWNRKRISN | | |
| CVADYSVLYN SASFSTFKCY GVSPTKLNDL CFTNVYADSF | | |
| VIRGDEVRQI APGQTGKIAD | | |
| YNYKLPDDFT GCVIAWNSNN LDSKVGGNYN YLYRLFRKSN | | |
| LKPFERDIST EIYQAGSTPC | | |
| NGVEGFNCYF PLQSYGFQPT NGVGYQPYRV VVLSFELLHA | | |
| PATVCGPKKS TNLVKNKCVN | | |
| FNFNGLTGTG VLTESNKKFL PFQQFGRDIA DTTDAVRDPQ | | |
| TLEILDITPC SFGGVSVITP | | |
| GTNTSNQVAV LYQDVNCTEV PVAIHADQLT PTWRVYSTGS | | |
| NVFQTRAGCL IGAEHVNNSY | | |
| ECDIPIGAGI CASYQTQTNS PRRARSVASQ SIIAYTMSLG | | |
| AENSVAYSNN SIAIPTNFTI | | |
| SVTTEILPVS MTKTSVDCTM YICGDSTECS NLLLQYGSFC | | |
| TQLNRALTGI AVEQDKNTQE | | |
| VFAQVKQIYK TPPIKDFGGF NFSQILPDPS KPSKRSFIED | | |
| LLFNKVTLAD AGFIKQYGDC | | |
| LGDIAARDLI CAQKFNGLTV LPPLLTDEMI AQYTSALLAG | | |
| TITSGWTFGA GAALQIPFAM | | |
| QMAYRFNGIG VTQNVLYENQ KLIANQFNSA IGKIQDSLSS | | |
| TASALGKLQD VVNQNAQALN | | |
| TLVKQLSSNF GAISSVLNDI LSRLDKVEAE VQIDRLITGR | | |
| LQSLQTYVTQ QLIRAAEIRA | | |
| SANLAATKMS ECVLGQSKRV DFCGKGYHLM SFPQSAPHGV | | |
| VFLHVTYVPA QEKNFTTAPA | | |
| ICHDGKAHFP REGVFVSNGT HWFVTQRNFY EPQIITTDNT | | |
| FVSGNCDVVI GIVNNTVYDP | | |
| LQPELDSFKE ELDKYFKNHT SPDVDLGDIS GINASVVNIQ | | |
| KEIDRLNEVA KNLNESLIDL | | |
| QELGKYEQYI KWPWYIWLGF IAGLIAIVMV TIMLCCMTSC | | |
| CSCLKGCCSC GSCCKFDEDD | | |

SEPVLKGVKL HYT

SARS-CoV-2 ORF PEPTIDE AND PROTEIN SEQUENCES

| | | | POSITION |
|---|---|---|---|
| | SEQ ID | | IN |
| SEQUENCE | NO: | PROTEIN | PROTEIN |
| RIFTIGTVTLKQGEI | 24 | Orf 3a | 6-20 |
| TVTLKQGEI | 25 | Orf 3a | 12-20 |
| MDLFMRIFTIGTVTLKQGEIKDATPSDFVRATATIPIQAS | 233 | Orf 3a | 1-275 |
| LPFGWLIVGVALLAVFQSASKIITLKKRWQLALSKGVH | | | (full-length) |
| FVCNLLLLFVTVYSHLLLVAAGLEAPFLYLYALVYFLQ | | | |
| SINFVRIIMRLWLCWKCRSKNPLLYDANYFLCWHTNC | | | |
| YDYCIPYNSVTSSIVITSGDGTTSPISEHDYQIGGYTEKW | | | |
| ESGVKDCVVLHSYFTSDYYQLYSTQLSTDTGVEHVTFF | | | |
| IYNKIVDEPEEHVQIHTIDGSSGVVNPVMEPIYDEPTTTT | | | |
| SVPL | | | |
| MAYCWRCTSCCFSERFQNHNPQKEMATSTLQGCSLCL | 185 | Orf3b | 1-57 |
| QLAVVVCNSLLTPFARCCWP | | | |
| MAYCWRCTSCCFSERFQNHN | 168 | Orf3b | 1-20 |
| CFSERFQNHNHNPQKEMATS | 169 | Orf3b | 11-28 |
| PQKEMATSTLQGCSLCLQLA | 170 | Orf3b | 21-40 |
| TLQGCSLCLQLAVVVCNSLL | 171 | Orf3b | 29-48 |
| VVVCNSLLTPFARCCWP | 172 | Orf3b | 41-57 |
| MKFLVFLGIITTVAAFHQECSLQSCTQHQPYVVDDPCPI | 186 | Orf8 | 1-121 |
| FIFYSKWYIRVGARKSAPLIELCVDEAGSKSPIQYIDIGN | | | (full-length) |
| YTVSCLPFTINCQEPKLGSLVVRCSFYEDFLEYHDVRVV | | | |
| LDFI | | | |
| MKFLVFLGIITTVAAFHQECS | 173 | Orf 8 | 1-21 |
| TVAAFHQECSLQSCTQHQPY | 174 | Orf8 | 12-31 |
| LQSCTQHQPYVVDDPCPIHF | 175 | Orf8 | 22-41 |
| VVDDPCPIHFYSKWYIRVGA | 176 | Orf 8 | 32-51 |
| YSKWYIRVGARKSAPLIELC | 177 | Orf 8 | 42-61 |
| RKSAPLIELCVDEAGSKSPI | 178 | Orf 8 | 52-71 |
| VDEAGSKSPIQYIDIGNYTV | 179 | Orf 8 | 62-81 |
| QYIDIGNYTVSCLPFTINCQ | 180 | Orf 8 | 72-91 |
| SCLPFTINCQEPKLGSLVVR | 181 | Orf 8 | 82-101 |
| EPKLGSLVVRCSFYEDFLEY | 182 | Orf 8 | 92-111 |
| CSFYEDFLEYHDVRVVLDFI | 183 | Orf 8 | 102-121 |
| SKWYIRVGARKSAPLIEL | 345 | Orf 8 | 43-60 |
| IDIGNYTVSCLPFTI | 346 | Orf 8 | 74-88 |
| FYEDFLEYHDVRVVL | 350 | Orf 8 | 104-118 |
| QYIDIGNYTVSCSPFTINCQ | 189 | Orf 8 | 72-91 |
| | | variant | |
| SCSPFTINCQEPKLGSLVVR | 190 | Orf 8 | 82-101 |
| | | variant | |
| GNYTVSCSPFTI | 347 | Orf 8 | 77-88 |
| | | variant | (L84S) |
| SQEPKLGSLVVR | 348 | Orf 8 | 90-101 |
| | | variant | (C90S) |
| RCSFYEDFLEYED | 340 | Orf 8 | 101-113 |
| | | variant | (H112E) |
| LGIITTVAAFHQECSLQSCT | 284 | Orf 8 | 7-26 |
| YSKWYIRVGAIKSAPLIELC | 285 | Orf 8 | 42-61 |
| | | variant | (R52I) |
| VDEAGSKSPIQSIDIGNYTV | 286 | Orf 8 | 62-81 |
| VDEAGSKSPIQCIDIGNYTV | 287 | Orf 8 | 62-81 |
| | | variant | (Y73) |
| QSIDIGNYTVSCLPFTINCQ | 288 | Orf 8 | 72-91 |
| QCIDIGNYTVSCLPFTINCQ | 289 | Orf 8 | 72-91 |
| | | variant | (Y73C) |
| MGYINVFAFPFTIYSLLLCRMNSRNYIAQVDVVNVNLT | 232 | Orf 10 | 1-38 |
| | | | (full-length) |
| MGYINVFAFPFTIYSLLLCR | 191 | Orf 10 | 1-20 |
| FTIYSLLLCRMNSRNYIAQV | 192 | Orf 10 | 11-30 |
| CRMNSRNYIAQVDVVNVNLT | 193 | Orf 10 | 19-38 |
| CLEASFNYLKSPNFSKLINII IWFLLLSVCL | 234 | Orf 1ab | 2210-3740 |
| GSLIYSTAAL GVLMSNLGMP SYCTGYREGY | | | |
| LNSTNVTIATYCTGSIPCSV CLSGLDSLDT YPSLETIQIT | | | |
| ISSFKWDLTAFGLVAEWFLAYILFTRFFYVLGLAAIMQL | | | |
| FFSYFAVHFIS NSWLMWLIIN LVQMAPISAM | | | |
| VRMYIFFASF YYVWKSYVHVVDGCNSSTCM | | | |
| MCYKRNRATR VECTTIVNGV RRSFYVYANG | | | |
| GKGFCKLHNW NCVNCDTFCAGSTFISDEVA | | | |
| RDLSLQFKRP INPTDQSSYI VDSVTVKNGS | | | |
| IHLYFDKAGQ KTYERHSLSHFVNLDNLRAN | | | |
| NTKGSLPINV IVFDGKSKCE ESSAKSASVY | | | |
| YSQLMCQPIL LLDQALVSDVGDSAEVAVKM | | | |
| FDAYVNTFSS TFNVPMEKLK TLVATAEAEL | | | |
| AKNVSLDNVL STFISAARQGFVDSDVETKD | | | |
| VVECLKLSHQ SDIEVTGDSC NNYMLTYNKV | | | |
| ENMTPRDLGA CIDCSARHINAQVAKSHNIA | | | |
| LIWNVKDFMS LSEQLRKQIR SAAKKNNLPF | | | |
| KLTCATTRQV VNVVTTKIALKGGKIVNNWL | | | |
| KQLIKVTLVF LFVAAIFYLI TPVHVMSKHT | | | |
| DFSSEIIGYK AIDGGVTRDIASTDTCFANK | | | |
| HADFDTWFSQ RGGSYTNDKA CPLIAAVITR | | | |
| EVGFVVPGLP GTILRTTNGDFLHFLPRVFS | | | |
| AVGNICYTPS KLIEYTDFAT SACVLAAECT | | | |
| IFKDASGKPV PYCYDTNVLEGSVAYESLRP | | | |
| DTRYVLMDGS IIQFPNTYLE GSVRVVTTFD | | | |
| SEYCRHGTCE RSEAGVCVSTSGRWVLNNDY | | | |
| YRSLPGVFCG VDAVNLLTNM FTPLIQPIGA | | | |
| LDISASIVAG GIVAIVVTCLAYYFMRFRRA | | | |
| FGEYSHVVAF NTLLFLMSFT VLCLTPVYSF | | | |
| LPGVYSVIYL YLTFYLTNDVSFLAHIQWMV | | | |
| MFTPLVPFWI TIAYIICIST KHFYWFFSNY | | | |
| LKRRVVFNGV SFSTFEEAALCTFLLNKEMY | | | |
| LKLRSDVLLP LTQYNRYLAL YNKYKYFSGA | | | |
| MDTTSYREAA CCHLAKALNDFSNSGSDVLY | | | |
| QPPQTSITSA VLQSGFRKMA FPSGKVEGCM | | | |
| VQVTCGTTTL NGLWLDDVVYCPRHVICTSE | | | |
| DMLNPNYEDL LIRKSNHNFL VQAGNVQLRV | | | |
| IGHSMQNCVL KLKVDTANPKTPKYKFVRIQ | | | |
| PGQTFSVLAC YNGSPSGVYQ CAMRPNFTIK | | | |
| GSFLNGSCGS VGFNIDYDCVSFCYMFIHMEL | | | |
| PTGVHAGTDL EGNFYGPFVD RQTAQAAGTD | | | |
| TTITVNVLAW LYAAVINGDRWFLNRFTTTL | | | |
| NDFNLVAMKY NYEPLTQDHV DILGPLSAQT | | | |
| GIAVLDMCAS LKELLQNGMNGRTILGSALL | | | |
| EDEFTPFDVV RQCSGVTFQS AVKRTIKGTH | | | |
| HWLLLTILTS LLVLVQSTQWSLFFFLYENA | | | |
| FLPFAMGIIA MSAFAMMFVK HKHAFLCLFL | | | |
| LPSLATVAYF NMVYMPASWVMRIMTWLDMV | | | |
| DTSLSGFKLK DCVMYASAVV LLILMTARTV | | | |
| YDDGARRVWT LMNVLTLVYKVYYGNALDQA | | | |
| ISMWALIISV | | | |
| CLEASFNYL | 39 | Orf 1ab | 2210-2218 |
| WLMWLIINL | 40 | Orf 1ab | 2363-2371 |
| ILLLDQALV | 41 | Orf 1ab | 2569-2577 |
| SACVLAAEC | 42 | Orf 1ab | 2911-2919 |
| SLPGVFCGV | 43 | Orf 1ab | 3013-3021 |
| TLMNVLTLV | 44 | Orf 1ab | 3710-3718 |
| SMWALIISV | 45 | Orf 1ab | 3732-3740 |
| VVLLSVLQQLRVESSSKLWA | 194 | Orf 1ab | 3870-3889 |
| RVESSSKLWAQCVQLHNDIL | 195 | Orf 1ab | 3880-3899 |
| QCVQLHNDILLAKDTTEAFE | 196 | Orf 1ab | 3890-3909 |
| LAKDTTEAFEKMVSLLSVLL | 197 | Orf 1ab | 3900-3919 |
| KMVSLLSVLLSMQGAVDINK | 198 | Orf 1ab | 3910-3929 |
| SMQGAVDINKLCEEMLDNRA | 199 | Orf 1ab | 3920-3939 |
| LCEEMLDNRATLQAIASEFS | 200 | Orf 1ab | 3930-3949 |
| TLQAIASEFSSLPSYAAFAT | 201 | Orf 1ab | 3940-3959 |
| SLPSYAAFATAQEAYEQAVA | 202 | Orf 1ab | 3950-3969 |
| AQEAYEQAVANGDSEVVLKK | 203 | Orf 1ab | 3960-3979 |
| NGDSEVVLKKLKKSLNVAKS | 204 | Orf 1ab | 3970-3989 |
| LKKSLNVAKSEFDRDAAMQR | 205 | Orf 1ab | 3980-3999 |
| EFDRDAAMQRKLEKMADQAM | 206 | Orf 1ab | 3990-4009 |
| KLEKMADQAMTQMYKQARSE | 207 | Orf 1ab | 4000-4019 |
| TQMYKQARSEDKRAKVTSAM | 208 | Orf 1ab | 4010-4029 |
| DKRAKVTSAMQTMLFTMLRK | 209 | Orf 1ab | 4020-4039 |
| QTMLFTMLRKLDNDALNNII | 210 | Orf 1ab | 4030-4049 |
| LDNDALNNIINNARDGCVPL | 211 | Orf 1ab | 4040-4059 |
| NNARDGCVPLNIIPLTTAAK | 212 | Orf 1ab | 4050-4069 |
| NIIPLTTAAKLMVVIPDYNT | 213 | Orf 1ab | 4060-4079 |
| LMVVIPDYNTYKNTCDGTTF | 214 | Orf 1ab | 4070-4089 |
| YKNTCDGTTFTYASALWEIQ | 215 | Orf 1ab | 4080-4099 |
| TYASALWEIQQVVDADSKIV | 216 | Orf 1ab | 4090-4109 |
| QVVDADSKIVQLSEISMDNS | 217 | Orf 1ab | 4100-4119 |
| QLSEISMDNSPNLAWPLIVT | 218 | Orf 1ab | 4110-4129 |
| PNLAWPLIVTALRANSAVKL | 219 | Orf 1ab | 4120-4139 |
| ALRANSAVKLQNNELSPVAL | 220 | Orf 1ab | 4130-4149 |
| QNNELSPVALRQMSCAAGTT | 221 | Orf 1ab | 4140-4159 |
| RQMSCAAGTTQTACTDDNAL | 222 | Orf 1ab | 4150-4169 |
| QTACTDDNALAYYNTTKGGR | 223 | Orf 1ab | 4160-4179 |
| AYYNTTKGGRFVLALLSDLQ | 224 | Orf 1ab | 4170-4189 |
| FVLALLSDLQDLKWARFPKS | 225 | Orf 1ab | 4180-4199 |
| DLKWARFPKSDGTGTIYTEL | 226 | Orf 1ab | 4190-4209 |
| DGTGTIYTELEPPCRFVTDT | 227 | Orf 1ab | 4200-4219 |
| EPPCRFVTDTPKGPKVKYLY | 228 | Orf 1ab | 4210-4229 |
| PKGPKVKYLYFIKGLNNLNR | 229 | Orf 1ab | 4220-4239 |
| KLWAQCVQLHNDILLAKDTTEAFEKMVSLLSVLLS | 230 | Orf 1ab | 3886-4239 |
| MQGAVDINKLCEEMLDNRATLQAIASEFSS | | | |
| LPSYAAFATAQEAYEQAVANGDSEVVLKKL | | | |
| KKSLNVAKSEFDRDAAMQRKLEKMADQAMT | | | |
| QMYKQARSEDKRAKVTSAMQTMLFTMLRKL | | | |
| DNDALNNIINNARDGCVPLNIIPLTTAAKL | | | |
| MVVIPDYNTYKNTCDGTTFTYASALWEIQQ | | | |
| VVDADSKIVQLSEISMDNSPNLAWPLIVTA | | | |
| LRANSAVKLQNNELSPVALRQMSCAAGTTQ | | | |
| TACTDDNALAYYNTTKGGRFVLALLSDLQD | | | |
| LKWARFPKSDGTGTIYTELEPPCRFVTDTP | | | |
| KGPKVKYLYFIKGLNNLNR | | | |
| VVLLSVLQQLRVESSSKLWAQCVQLHNDILLAKDTTEA | 231 | Orf 1ab | 3870-4239 |
| FEKMVSLLSVLLSMQGAVDINKLCEEMLDNRATLQAIA | | | |
| SEFSSLPSYAAFATAQEAYEQAVANGDSEVVLKKLKKS | | | |
| LNVAKSEFDRDAAMQRKLEKMADQAMTQMYKQARS | | | |
| EDKRAKVTSAMQTMLFTMLRKLDNDALNNIINNARDG | | | |
| CVPLNIIPLTTAAKLMVVIPDYNTYKNTCDGTTFTYASA | | | |
| LWEIQQVVDADSKIVQLSEISMDNSPNLAWPLIVTALR | | | |
| ANSAVKLQNNELSPVALRQMSCAAGTTQTACTDDNAL | | | |
| AYYNTTKGGRFVLALLSDLQDLKWARFPKSDGTGTIY | | | |
| TELEPPCRFVTDTPKGPKVKYLYFIKGLNNLNR | | | |
| MAYCWRCTSCCFSERFQNHNPQ | 341 | Orf3b | 1-22 |
| CFSERFQNHNPQKEMATSTL | 342 | Orf3b | 11-30 |
| VWCNSLLTPFARCCWP | 343 | Orf3b | 42-57 |
| | | | (V43W) |
| TVMFHQECSLQSCTQHQPY | 344 | Orf8 | 12-31 |
| | | | (A14M/ |
| | | | ΔA15) |

SARS-CoV-2 PROTEIN M AND N SEQUENCES

| | SEQ ID | | POSITION IN |
|---|---|---|---|
| PEPTIDE SEQUENCE | NO: | PROTEIN | PROTEIN |
| TLACFVLAAV | 26 | M | 61-70 |
| GLMWLSYFI | 27 | M | 89-97 |
| FILRIAGFIHL | 28 | M | 148-156 |
| ALNTPKDHI | 29 | N | 138-146 |
| LQLPQGTTL | 30 | N | 159-167 |
| GDAALALLLL | 31 | N | 215-224 |
| LALLLLDRL | 32 | N | 219-227 |
| LLLDRLNQL | 33 | N | 222-230 |
| RLNQLESKM | 34 | N | 226-234 |
| TKAYNVTQAF | 35 | N | 265-274 |
| GMSRIGMEV | 36 | N | 316-324 |
| MEVTPSGTWL | 37 | N | 322-331 |
| NFKDQVILL | 38 | N | 345-353 |

SARS-CoV-2 PROTEIN M AND N SEQUENCES (Wuhan-HU-1, Accession MN988668.1)

| FULL SEQUENCE M PROTEIN | SEQ ID NO: |
|---|---|
| MADSNGTITVEELKKLLEQWNLVIGFLFLTWICLLQFAYANRNR | 187 |
| FLYIIKLIFLWLLWPVTLACFVLAAVYRINWITGGIAIAMACLVGL | |
| MWLSYFIASFRLFARTRSMWSFNPETNILLNVPLHGTILTRPLLESE | |
| LVIGAVILRGHLRIAGHHLGRCDIKDLPKEITVATSRTLSYYKLGA | |
| SQRVAGDSGFAAYSRYRIGNYKLNTDHSSSSDNIALLVQ | |
| FULL SEQUENCE N PROTEIN | SEQ ID NO: |
| MSDNGPQNQRNAPRITFGGPSDSTGSNQNGERSGARSKQRRPQG | 188 |
| LPNNTASWFTALTQHGKEDLKFPRGQGVPINTNSSPDDQIGYYRR | |
| ATRRIRGGDGKMKDLSPRWYFYYLGTGPEAGLPYGANKDGIIWV | |
| ATEGALNTPKDHIGTRNPANNAAIVLQLPQGTTLPKGFYAEGSRG | |
| GSQASSRSSSRSRNSSRNSTPGSSRGTSPARMAGNGGDAALALLL | |
| LDRLNQLESKMSGKGQQQQGQTVTKKSAAEASKKPRQKRTATK | |
| AYNVTQAFGRRGPEQTQGNFGDQELIRQGTDYKHWPQIAQFAPS | |
| ASAFFGMSRIGMEVTPSGTWLTYTGAIKLDDKDPNFKDQVILLNK | |
| HIDAYKTFPPTEPKKDKKKKADETQALPQRQKKQQTVTLLPAAD | |
| LDDFSKQLQQSMSSADSTQA | |

### Methods of the Disclosure

The present disclosure provides methods for detecting cell-mediated immunity to SARS-CoV-2 in a subject, methods for detecting a cell-mediated immune response to SARS-CoV-2 in a subject, methods for determining if a vaccination to SARS-CoV-2 elicits a cell-mediated immune response in a subject and methods for detecting a SARS-CoV-2 infection in a subject, the methods comprising administering to the skin of the subject one or more peptides of the disclosure and detecting the presence of an immune reaction in the subject by inspecting the skin in the area of administration.

In one aspect, the present disclosure provides methods for detecting cell-mediated immunity to SARS-CoV-2 in a subject, the method comprising administering to the skin of the subject one or more peptides, the peptides being selected from the group consisting of peptides comprising any one of the amino acid sequences set forth in SEQ ID NO: 1-183 and inspecting the skin region in the area of the administration for an immune reaction, wherein the presence of an immune reaction is indicative of the subject having developed cell-mediated immunity to SARS-COV-2.

In one aspect, the present disclosure provides methods for detecting cell-mediated immunity to SARS-CoV-2 in a subject, the method comprising administering to the skin of the subject one or more peptides, the peptides being selected from the group consisting of peptides comprising any one of the amino acid sequences set forth in SEQ ID NO: 1-183 and 189-231, and preferably combinations of several of them and inspecting the skin region in the area of the administration for an immune reaction, wherein the presence of an immune reaction is indicative of the subject having developed cell-mediated immunity to SARS-COV-2.

In one aspect, the present disclosure provides methods for detecting cell-mediated immunity to SARS-CoV-2 in a subject, the method comprising administering to the skin of the subject one or more peptides, the peptides being selected from the group consisting of peptides comprising any one of the amino acid sequences set forth in SEQ ID NO: 1-358, and preferably combinations of several of them and inspecting the skin region in the area of the administration for an immune reaction, wherein the presence of an immune reaction is indicative of the subject having developed cell-mediated immunity to SARS-COV-2.

In one aspect, the present disclosure provides methods for detecting cell-mediated immunity to SARS-CoV-2 in a subject, the method comprising administering to the skin of the subject one or more peptides, the peptides being selected from the group consisting of peptides comprising any one of the amino acid sequences set forth in SEQ ID NO: 1-183, 189-231 and 234-358, and preferably combinations of several of them and inspecting the skin region in the area of the administration for an immune reaction, wherein the presence of an immune reaction is indicative of the subject having developed cell-mediated immunity to SARS-COV-2.

In one aspect, the present disclosure provides methods for detecting cell-mediated immunity to SARS-CoV-2 in a subject, the method comprising administering to the skin of the subject one or more peptides, the peptides being selected from the group consisting of peptides comprising any one of the amino acid sequences set forth in SEQ ID NO: 1-183, 189-232, 294, 299, 300, 303, 307-312, 317, 319, 320, 324-327, 333 and 335-337, 341 and 342, and preferably combinations of several of them, and inspecting the skin region in the area of the administration for an immune reaction, wherein the presence of an immune reaction is indicative of the subject having developed cell-mediated immunity to SARS-COV-2.

In one aspect, the present disclosure provides methods for detecting cell-mediated immunity to SARS-CoV-2 in a subject, the method comprising administering to the skin of the subject one or more peptides, the peptides being selected from the group consisting of peptides being characterized by any one of the amino acid sequences set forth in SEQ ID NO: 1-104, 109-138, 140-183, 189-2312, 235-289, 294, 299, 303, 307-312, 317, 319, 320, 324-327, 333, 335-337, 341, 342 and 345-350, and preferably combinations of several of them, and inspecting the skin region in the area of the administration for an immune reaction, wherein the presence of an immune reaction is indicative of the subject having developed cell-mediated immunity to SARS-COV-2.

In one aspect, the present disclosure provides methods for detecting cell-mediated immunity to SARS-CoV-2 in a subject, the method comprising administering to the skin of the subject one or more peptides, the peptides being selected from the group consisting of peptides being characterized by any one of the amino acid sequences set forth in SEQ ID NO: 1-14, 16-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-168, 170, 171, 173, 175, 176, 178, 179, 181, 190, 235-248, 250-255, 257-261, 266, 268, 265, 272, 274, 275, 277-283, 290-306, 308-322, 324, 325, 328, 328, 330-334, 337-340, and 342-358, and preferably combinations of several of them, and inspecting the skin region in the area of the administration for an immune reaction, wherein the presence of an immune reaction is indicative of the subject having developed cell-mediated immunity to SARS-COV-2.

In another aspect, the present disclosure provides methods for detecting a cell-mediated immune response to SARS-CoV-2 in a subject, the method comprising administering to the skin of the subject one or more peptides, the peptides being selected from the group consisting of peptides comprising any one of the amino acid sequences set forth in SEQ ID NO: 1-183 and inspecting the skin region in the area of the administration for an immune reaction, wherein the presence of an immune reaction is indicative of the subject having developed a cell-mediated immune response to SARS-CoV-2.

In another aspect, the present disclosure provides methods for detecting a cell-mediated immune response to SARS-CoV-2 in a subject, the method comprising administering to the skin of the subject one or more peptides, the peptides being selected from the group consisting of peptides comprising any one of the amino acid sequences set forth in SEQ ID NO: 1-183, and preferably combinations of several of them, and inspecting the skin region in the area of the administration for an immune reaction, wherein the presence of an immune reaction is indicative of the subject having developed a cell-mediated immune response to SARS-CoV-2.

In another aspect, the present disclosure provides methods for detecting a cell-mediated immune response to SARS-CoV-2 in a subject, the method comprising administering to the skin of the subject one or more peptides, the peptides being selected from the group consisting of peptides comprising any one of the amino acid sequences set forth in SEQ ID NO: 1-183 and 189-231, and preferably combinations of several of them, and inspecting the skin region in the area of the administration for an immune reaction, wherein the presence of an immune reaction is indicative of the subject having developed a cell-mediated immune response to SARS-CoV-2.

In another aspect, the present disclosure provides methods for detecting a cell-mediated immune response to SARS-CoV-2 in a subject, the method comprising administering to the skin of the subject one or more peptides, at least one, at least five, at least ten, at least fifteen, at least twenty, at least twenty-five, at least thirty of the one or more peptides being selected from the group consisting of peptides comprising any one of the amino acid sequences set forth in SEQ ID NO: 1-183, 189-231 and 234-358, and preferably combinations of several of them, and inspecting the skin region in the area of the administration for an immune reaction, wherein the presence of an immune reaction is indicative of the subject having developed a cell-mediated immune response to SARS-CoV-2.

In another aspect, the present disclosure provides methods for detecting a cell-mediated immune response to SARS-CoV-2 in a subject, the method comprising administering to the skin of the subject one or more peptides, at least one, at least five, at least ten, at least fifteen, at least twenty, at least twenty-five, at least thirty of the one or more peptides being selected from the group consisting of peptides comprising any one of the amino acid sequences set forth in SEQ ID NO: 1-183, 189-232, 294, 299, 300, 303, 307-312, 317, 319, 320, 324-327, 333 and 335-337, 341 and 342, and preferably combinations of several of them, and inspecting the skin region in the area of the administration for an immune reaction, wherein the presence of an immune reaction is indicative of the subject having developed a cell-mediated immune response to SARS-CoV-2.

In another aspect, the present disclosure provides methods for detecting a cell-mediated immune response to SARS-CoV-2 in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by any one of the amino acid sequences set forth in SEQ ID NO: 1-104, 109-138, 140-183, 189-231, 235-289, 294, 299, 303, 307-312, 317, 319, 320, 324-327, 333, 335-337, 341, 342 and 345-350, and preferably combinations of several of them, and inspecting the skin region in the area of the administration for an immune reaction, wherein the presence of an immune reaction is indicative of the subject having developed a cell-mediated immune response to SARS-CoV-2.

In another aspect, the present disclosure provides methods for detecting a cell-mediated immune response to SARS-CoV-2 in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by any one of the amino acid sequences set forth in SEQ ID NO: 1-14, 16-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-168, 170, 171, 173, 175, 176, 178, 179, 181, 190, 235-248, 250-255, 257-261, 266, 268, 265, 272, 274, 275, 277-283, 290-306, 308-322, 324, 325, 328, 328, 330-334, 337-340, and 342-358, and preferably combinations of several of them, and inspecting the skin region in the area of the administration for an immune reaction, wherein the presence of an immune reaction is indicative of the subject having developed a cell-mediated immune response to SARS-CoV-2.

In another aspect, the present disclosure provides methods for determining if a potential or candidate vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides comprising any one of the amino acid sequences set forth in SEQ ID NO: 1-183 and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if a potential or candidate vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides comprising any one of the amino acid sequences set forth in SEQ ID NO: 1-358 and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if a potential or candidate vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides comprising any one of the amino acid sequences set forth in SEQ ID NO: 1-183, 189-231 and 234-358 and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if a potential or candidate vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by any one of the amino acid sequences set forth in SEQ ID NO: 1-104, 109-138, 140-183, 189-231, 235-289, 294, 299, 303, 307-312, 317, 319, 320, 324-327, 333, 335-337, 341, 342 and 345-350 and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if a potential or candidate vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides comprising any one of the amino acid sequences set forth in SEQ ID NO: 1-14, 16-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-168, 170, 171, 173, 175, 176, 178, 179, 181, 190, 235-248, 250-255, 257-261, 266, 268, 265, 272, 274, 275, 277-283, 290-306, 308-322, 324, 325, 328, 328, 330-334, 337-340, and 342-358 and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if a potential or candidate vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides comprising any one of the amino acid sequences set forth in SEQ ID NO: 1-183, and preferably combinations of several of them, and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if a potential or candidate vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides comprising any one of the amino acid sequences set forth in SEQ ID NO: 1-183 and 189-231, and preferably combinations of several of them, and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if a potential or candidate vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides comprising any one of the amino acid sequences set forth in SEQ ID NO: 1-358, and preferably combinations of several of them, and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if a potential or candidate vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides comprising any one of the amino acid sequences set forth in SEQ ID NO: 1-183, 189-231 and 234-358, and preferably combinations of several of them, and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if a potential or candidate vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by any one of the amino acid sequences set forth in SEQ ID NO: 1-104, 109-138, 140-183, 189-231, 235-289, 294, 299, 303, 307-312, 317, 319, 320, 324-327, 333, 335-337, 341, 342 and 345-350, and preferably combinations of several of them, and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if a potential or candidate vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides comprising any one of the amino acid sequences set forth in SEQ ID NO: 1-14, 16-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-168, 170, 171, 173, 175, 176, 178, 179, 181, 190, 235-248, 250-255, 257-261, 266, 268, 265, 272, 274, 275, 277-283, 290-306, 308-322, 324, 325, 328, 328, 330-334, 337-340, and 342-358, and preferably combinations of several of them, and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if a potential or candidate vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides comprising any one of the amino acid sequences set forth in SEQ ID NO: 1-183, 189-232, 294, 299, 300, 303, 307-312, 317, 319, 320, 324-327, 333 and 335-337, 341 and 342 and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if a potential or candidate vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by any one of the amino acid sequences set forth in SEQ ID NO: 1-104, 109-138, 140-183, 189-231, 235-289, 294, 299, 303, 307-312, 317, 319, 320, 324-327, 333, 335-337, 341, 342 and 345-350 and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if an approved vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by any one of the amino acid sequences set forth in SEQ ID NO: 1-183 and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if an approved vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by any one of the amino acid sequences set forth in SEQ ID NO: 1-358 and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if an approved vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by any one of the amino acid sequences set forth in SEQ ID NO: 1-183, 189-231 and 234-358 and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if an approved vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by any one of the amino acid sequences set forth in SEQ ID NO: 1-104, 109-138, 140-183, 189-231, 235-289, 294, 299, 303, 307-312, 317, 319, 320, 324-327, 333, 335-337, 341, 342 and 345-350 and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if an approved vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by any one of the amino acid sequences set forth in SEQ ID NO: 1-14, 16-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-168, 170, 171, 173, 175, 176, 178, 179, 181, 190, 235-248, 250-255, 257-261, 266, 268, 265, 272, 274, 275, 277-283, 290-306, 308-322, 324, 325, 328, 328, 330-334, 337-340, and 342-358 and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if an approved vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by any one of the amino acid sequences set forth in SEQ ID NO: 1-183, and preferably combinations of several of them, and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if an approved vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by any one of the amino acid sequences set forth in SEQ ID NO: 1-183, 189-231 and 234-358, and preferably combinations of several of them, and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if an approved vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by any one of the amino acid sequences set forth in SEQ ID NO: 1-104, 109-138, 140-183, 189-231, 235-289, 294, 299, 303, 307-312, 317, 319, 320, 324-327, 333, 335-337, 341, 342 and 345-350, and preferably combinations of several of them, and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if an approved vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by any one of the amino acid sequences set forth in SEQ ID NO: 1-14, 16-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-168, 170, 171, 173, 175, 176, 178, 179, 181, 190, 235-248, 250-255, 257-261, 266, 268, 265, 272, 274, 275, 277-283, 290-306, 308-322, 324, 325, 328, 328, 330-334, 337-340, and 342-358, and preferably combinations of several of them, and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if an approved vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides comprising any one of the amino acid sequences set forth in SEQ ID NO: 1-183 and 189-231 and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if an approved or actual vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by any one of the amino acid sequences set forth in SEQ ID NO: 1-183, 189-232, 294, 299, 300, 303, 307-312, 317, 319, 320, 324-327, 333 and 335-337, 341 and 342 and inspecting the skin region in the area of the administration for an immune reaction, wherein the presence of an immune reaction is indicative of the vaccine eliciting a cell-mediated immune response to SARS-CoV-2 in the subject.

In another aspect, the present disclosure provides methods for determining if an approved vaccine to SARS-CoV-2 elicits a cell-mediated immune response in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by any one of the amino acid sequences set forth in SEQ ID NO: 1-104, 109-138, 140-183, 189-231, 235-289, 294, 299, 303, 307-312, 317, 319, 320, 324-327, 333, 335-337, 341, 342 and 345-350 and inspecting the skin region in the area of the administration for an immune reaction, wherein the presence of an immune reaction is indicative of the vaccine eliciting a cell-mediated immune response to SARS-CoV-2 in the subject.

Any one, any mixture or any combination of the peptides disclosed in the present disclosure may be used in the methods disclosed herein.

In some embodiments, the one or more peptides are selected from the group consisting of peptides being characterized by the amino acid sequences set forth in SEQ ID NO: 1-183. In some embodiments, a combination of more than one peptide (e.g., 2, 4, 5, 10, 15, etc.) is used in the methods disclosed herein.

In some embodiments, the one or more peptides are selected from the group consisting of peptides being characterized by the amino acid sequences set forth in SEQ ID NO: 1-183 and 189-231. In some embodiments, a combination of more than one peptide (e.g., 2, 4, 5, 10, 15, etc.) is used in the methods disclosed herein.

In some embodiments, the one or more peptides are selected from the group consisting of peptides being characterized by the amino acid sequences set forth in SEQ ID NO: 1-358. In some embodiments, a combination of more than one peptide (e.g., 2, 4, 5, 10, 15, etc.) is used in the methods disclosed herein.

In some embodiments, the one or more peptides are selected from the group consisting of peptides being characterized by the amino acid sequences set forth in SEQ ID NO: 1-183, 189-231 and 234-358. In some embodiments, a combination of more than one peptide (e.g., 2, 4, 5, 10, 15, etc.) is used in the methods disclosed herein.

In some embodiments, the one or more peptides are selected from the group consisting of peptides being characterized by the amino acid sequences set forth in SEQ ID NO: 1-183, 189-232, 294, 299, 300, 303, 307-312, 317, 319, 320, 324-327, 333 and 335-337, 341 and 342. In some embodiments, a combination of more than one peptide (e.g., 2, 4, 5, 10, 15, etc.) is used in the methods disclosed herein.

In some embodiments, the one or more peptides are selected from the group consisting of peptides being characterized by the amino acid sequences set forth in SEQ ID NO: 1-104, 109-138, 140-183, 189-231, 235-289, 294, 299, 303, 307-312, 317, 319, 320, 324-327, 333, 335-337, 341, 342 and 345-350. In some embodiments, a combination of more than one peptide (e.g., 2, 4, 5, 10, 15, etc.) is used in the methods disclosed herein.

In some embodiments, the one or more peptides are selected from the group consisting of peptides being characterized by the amino acid sequences set forth in SEQ ID NO: 1-14, 16-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-168, 170, 171, 173, 175, 176, 178, 179, 181, 190, 235-248, 250-255, 257-261, 266, 268, 265, 272, 274, 275, 277-283, 290-306, 308-322, 324, 325, 328, 328, 330-334, 337-340, and 342-358. In some embodiments, a combination of more than one peptide (e.g., 2, 4, 5, 10, 15, etc.) is used in the methods disclosed herein.

In some embodiments, the one or more peptides are administered or applied to the skin (i.e., cutaneous administration). In some embodiments, the one or more peptides may be administered to any of the skin layers (i.e., epidermis, dermis or hypodermis). Methods for cutaneous administration of peptides are known in the art. In some embodiments, the administration of the one or more peptides is intradermal, intraepidermal, subcutaneous, transdermal, percutaneous or the like. In some embodiments, the administration is intradermal. In some embodiments, the one or more peptides are administered from about 0.5 mm to about 2 mm within the dermis or from about 1 mm to about 2 mm within the dermis, such that the one or more peptides are administered into the dermis layer of the subject. In some embodiments, the administration is made by delivering the one or more peptides into the epidermis and upper layers of the dermis. In some embodiments, the site of administration in the subject's skin is the ventral surface of the forearm or the upper back under the scapula of the subject.

In some embodiments, the one or more peptides are administered as a composition comprising a pharmaceutically acceptable buffer. Suitable carriers and their formulations are described, for example, in Remington's Pharmaceutical Sciences by E. W. Martin. In some embodiments, one or more peptides are provided in a dosage form that is suitable for cutaneous administration. In some embodiments, at least one or more of the peptides are present in the form of a lyophilizate. In some embodiments, at least one or more of the peptides are present in a solution. In some embodiments, at least one or more of the peptides are administered in a solution. In some embodiments, one or more peptides are dissolved in a solvent or reconstituted in a solvent if they are in the form of a lyophilizate. In some embodiments, the one or more peptides are dissolved in a solvent or reconstituted before administration to the subject. In some embodiments, the solvent is selected from the group consisting of glycerol, water for injection, a phosphate buffered saline, a sodium phosphate buffer, a Tris buffer, a borate buffer, a succinate buffer, a histidine buffer, a citrate buffer, a potassium phosphate buffer and a mannitol solution. In some embodiments, the one or more peptides solvent further comprises at least one preservative. In some embodiments, the preservative is selected from the group consisting of phenol, m-cresol, thiomersal, 2-phenoxyethanol and 8-hydroxy quinoline. In some embodiments, the solution further comprises at least one stabilizer. In some embodiments, the stabilizer is a non-ionic surfactant. In some embodiments, the non-ionic surfactant is Polysorbate 80, Polysorbate 20, Triton™ X-100, polyoxyethylene sorbitan, fatty acid esters, Poloxamer, polyoxyl-40-stearate and other polyoxyethylene stearates, glycerol monostearate, macrogol-8-stearat, macrogol cetostearylether 20, polyoxyethylene alkyl ethers, sorbitan monostearate and other sorbitan monoesters, polyoxyethylene castor oil derivatives, sodium lauryl sulfate, cetylpyridinium chloride or the like. In some embodiments, the one or more peptide solution comprises a pH between about 5.0 and about 9.5. In some embodiments, the one or more peptide solution comprises a pH between about 6.0 and about 8.0. In some embodiments, the one or more peptide solution comprises a pH of about 7.0.

In some embodiments, the one or more peptides are administered simultaneously. In some embodiments, the one or more peptides are administered sequentially. In some embodiments, at least some of the peptides are administered simultaneously, while others are administered sequentially.

In some embodiments, the one or more peptides are administered to the skin of the subject in an amount effective to elicit an immune reaction in the area of the administration. In some embodiments, the amount administered of each of the one or more peptides is from about 0.01 μg to about 1000 μg, from about 1 μg to about 800 μg, from about 5 μg to about 500 μg, from about 10 μg to about 100 μg, from about 1 μg to about 100 μg, from about 0.1 μg to about 100 μg, from about 0.01 μg to about 50 μg. In some embodiments, each of the one or more peptides are administered to the skin in an amount of about 0.01, 0.1, 1, 10, 15, 20, 25, 30, 35, 40, 45, 50, 55, 60, 65, 70, 75, 80, 85, 90, 95 or 100 μg. In some embodiments, the combination of the peptides are administered to the skin in a total amount of about 0.01, 0.1, 1, 10, 15, 20, 25, 30, 35, 40, 45, 50, 55, 60, 65, 70, 75, 80, 85, 90, 95 or 100 μg. In some embodiments, the one or more peptides are comprised within a solution and the volume administered is less than about 1 ml, less than about 0.5 ml, less than about 0.4 ml or less than about 0.2 ml. In some embodiments, the volume administered is about 0.1 ml, about 0.25 ml, about 0.5 ml or about 1 ml.

In some embodiments, the amount of each peptide in a peptide combination is the same. In some embodiments, the amount of each peptide in the combination is not the same and a skilled in the art will know how to adjust the amount to be used of each peptide in the methods of the disclosure.

In some embodiments, upon the administration of the one or more peptides to the skin of the subject, an immune reaction corresponding to a cell-mediated immune response is triggered. In some embodiments, the immune reaction observed is a delayed type hypersensitivity (DTH) reaction. In some embodiments, the immune reaction is detected by inspecting the skin region in the area of the administration. In some embodiments, the inspection of the skin region is performed visually by an individual. In some embodiments, the inspection of the skin region is performed by an automatic device.

In some embodiments, upon the administration of the one or more peptides to the skin of the subject, the skin region in the area of the administration is swelled as a consequence of the immune reaction, for example, an induration (a localized hardening of soft tissue) is produced at the area of the administration. In some embodiments, the swelling at the skin region is observed within about 12 to 72 hours, within about 24 to 72 hours or within about 24 to 48 hours after the administration. In some embodiments, the peak of the swelling at the skin region is observed within about 12 to 72 hours, within about 24 to 72 hours or within about 24 to 48 hours after the administration. In some embodiments, an induration or reaction of more than about 2 mm, more than about 3 mm, more than about 5 mm, more than about 7 mm, more than about 10 mm, more than about 15 mm or more, in diameter is considered an immune reaction or a positive immune reaction in the subject. In some embodiments, the immune reaction is measured visually by an individual. In some embodiments, the immune reaction is measured by using a laser Doppler imaging (Harrison, et al., Physiol Meas. 1993 August; 14(3):241-52), using a hand-held spectrophotometer to measure the DTH reaction (Chambers, et al., Skin Res Technol. 2002 May; 8(2):89-93) or by ultrasonographic measurement (Ciftci, et al., Reading. Infect Dis Clin Pract (Baltim Md.) 2005; 13:20-23). In some embodiments, the skin test is read or inspected at about 48 hours after administration and measurements are made across two diameters in the indurated area. In some embodiments, the mean of the longest and midpoint orthogonal diameters of the indurated area is reported as the DTH reaction. For example, a reaction that is about 10 mm (longest diameter) by about 8 mm (midpoint orthogonal diameter) has a sum of about 18 mm and a mean of about 9 mm. The DTH response is therefore about 9 mm.

In some embodiments, upon the administration of the one or more peptides to the skin of the subject, the skin region in the area of the administration is swelled as a consequence of the immune reaction, for example, an induration (a localized hardening of soft tissue) is produced at the area of the administration. In some embodiments, the swelling at the skin region is observed within about 12 to 72 hours, within about 24 to 72 hours, within about 24 to 48 hours, within about 24 to 96 hours, within about 48 to 96 hours or within about 48 to 72 hours after the administration. In some embodiments, the peak of the swelling at the skin region is observed within about 12 to 72 hours, within about 24 to 72 hours, within about 24 to 48 hours, within about 24 to 96 hours, within about 48 to 96 hours or within about 48 to 72 hours after the administration. In some embodiments, the skin test is read or inspected at about 72 hours after administration and measurements are made across two diameters in the indurated area. In some embodiments, the skin test is read or inspected at about 72 hours after administration and measurements are made across two diameters in the indurated area. In some embodiments, the skin test is read or inspected at about 96 hours after administration and measurements are made across two diameters in the indurated area. In some embodiments, the skin test is read or inspected at about 96 hours after administration and measurements are made across two diameters in the indurated area.

In some embodiments, an induration or reaction of less than about 2 mm, less than about 3 mm, less than about 4 mm, or less than about 5 mm in diameter is considered a negative immune reaction or a negative immune reaction in the subject. In some embodiments, the administered negative control causes no induration or reaction to the skin of the subject. In some embodiments, the induration or reaction of the administered negative control is weaker than the administered one or more peptides to the skin of the subject.

In some embodiments, the observed immune reaction in the subject is indicative of the subject having developed cell-mediated immunity to SARS-CoV-2. In some embodiments, the observed immune reaction in the subject is indicative of the subject having developed a cell-mediated immune response to SARS-CoV-2. In some embodiments, the observed immune reaction in the subject is indicative of a potential vaccine or an approved vaccine to SARS-CoV-2 eliciting a cell-mediated immune response in the subject. In some embodiments, the observed immune reaction in the subject is indicative of the subject having an active SARS-CoV-2 infection.

In some embodiments, the one or more peptides are administered to the skin with a syringe, a microneedle patch, a lancet or the like. In some embodiments, a 26 to 30-gauge needle is used for the administration. In some embodiments, the one or more peptides are administered with a microneedle patch. See, e.g., Mandal et al., Sci. Transl. Med. 10, eaar2227 (2018) and McCormick T, Shearer W. 2006. Delayed-Type Hypersensitivity Skin Testing, p 234-240, incorporated by reference herein in their entireties.

In one aspect, the present disclosure provides methods to stratify participants in vaccine trials. In some embodiments, the participant grouping is determined by clinical history, nasal swabs, and/or serology/PCR testing. In some embodiments, the participants are SARS-CoV-2 naïve, without history of COVID-19 and have a negative serology/PCR test for SARS-CoV-2 infection. In some embodiments, the participants have had an acute SARS-CoV-2 infection. In some embodiments, the participants are asymptomatic and have a positive serology/PCR test for SARS-CoV-2 infection. In some embodiments, the participants are convalescent with resolved SARS-CoV-2 infection. In some embodiments, the present disclosure provides methods to stratify participants in vaccine trials by immune status. In some embodiments, the present disclosure provides methods to stratify participants in COVID-19 vaccine trials by immune status.

In one aspect, the present disclosure provides methods of using surrogate marker in vaccine trials to evaluate cell-mediated immune response to the vaccines being evaluated.

In one aspect, the present disclosure provides methods to measure durability of the cell-mediated immune response to vaccines following natural infection or vaccination. In some embodiments, the durability of the cell-mediated immune response lasts for months. In some embodiments, the durability of the cell-mediated immune response lasts for years.

### Kits of the Disclosure

In one aspect, the disclosure relates to a kit for detecting a cell-mediated immune response to SARS-CoV-2 in a subject, said kit comprising one, five, ten, fifteen, twenty, twenty-five, thirty or more peptides, the peptides being selected from the group consisting of peptides comprising the amino acid sequences set forth in SEQ ID NO: 1-183 and instructions for its use.

In one aspect, the disclosure relates to a kit for detecting a cell-mediated immune response to SARS-CoV-2 in a subject, said kit comprising one, five, ten, fifteen, twenty, twenty-five, thirty or more peptides, the peptides being selected from the group consisting of peptides being characterized by the amino acid sequences set forth in SEQ ID NO: 1-183 and 189-231 and instructions for its use.

In one aspect, the disclosure relates to a kit for detecting a cell-mediated immune response to SARS-CoV-2 in a subject, said kit comprising one or more peptides, the peptides being selected from the group consisting of peptides being characterized by the amino acid sequences set forth in SEQ ID NO: 1-358 and instructions for its use.

In one aspect, the disclosure relates to a kit for detecting a cell-mediated immune response to SARS-CoV-2 in a subject, said kit comprising one, five, ten, fifteen, twenty, twenty-five, thirty or more peptides, the peptides being selected from the group consisting of peptides being characterized by the amino acid sequences set forth in SEQ ID NO: 1-183, 189-231 and 234-358 and instructions for its use.

In one aspect, the disclosure relates to a kit for detecting a cell-mediated immune response to SARS-CoV-2 in a subject, said kit comprising one, five, ten, fifteen, twenty, twenty-five, thirty or more peptides, the peptides being selected from the group consisting of peptides being characterized by the amino acid sequences set forth in SEQ ID NO: 1-183, 189-232, 294, 299, 300, 303, 307-312, 317, 319, 320, 324-327, 333 and 335-337, 341 and 342 and instructions for its use.

In one aspect, the disclosure relates to a kit for detecting a cell-mediated immune response to SARS-CoV-2 in a subject, said kit comprising one, five, ten, fifteen, twenty, twenty-five, thirty or more peptides, the peptides being selected from the group consisting of peptides being characterized by the amino acid sequences set forth in SEQ ID NO: 1-104, 109-138, 140-183, 189-231, 235-289, 294, 299, 300, 303, 307-312, 317, 319, 320, 324-327, 333, 335-337, 341, 342 and 345-350 and instructions for its use.

In one aspect, the disclosure relates to a kit for detecting a cell-mediated immune response to SARS-CoV-2 in a subject, said kit comprising one, five, ten, fifteen, twenty, twenty-five, thirty or more peptides, the peptides being selected from the group consisting of peptides being characterized by the amino acid sequences set forth in SEQ ID NO: 1-14, 16-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-168, 170, 171, 173, 175, 176, 178, 179, 181, 190, 235-248, 250-255, 257-261, 266, 268, 265, 272, 274, 275, 277-283, 290-306, 308-322, 324, 325, 328, 328, 330-334, 337-340, and 342-358 and instructions for its use.

In one aspect, the disclosure relates to a kit for detecting a cell-mediated immune response to SARS-CoV-2 in a subject, said kit comprising one, five, ten, fifteen, twenty, twenty-five, thirty or more peptides, the peptides being selected from the group consisting of peptides being characterized by the amino acid sequences set forth in SEQ ID NO: 1-10, 12-14, 17, 18, 20-25, 27, 28, 30-39, 41, 43, 44 and 292, and optionally one or more sequences characterized by the amino acid sequences set forth in SEQ ID NO: 11, 16, 19, 26, 29, 40, 42, and 45 and instructions for its use.

In one aspect, the disclosure relates to a kit for detecting a cell-mediated immune response to SARS-CoV-2 in a subject, said kit comprising one, five, ten, fifteen, twenty, twenty-five, thirty or more peptides, the peptides being selected from the group consisting of peptides being characterized by the amino acid sequences set forth in SEQ ID NO: 47, 48, 50-55, 59, 60, 62-64, 66-70, 72, 74, 75, 77, 79-81, 83, 84, 86, 87, 90-94, 96, 99-101, 103, 110, 115-125, 129, 131-133, 136-138, 140, 142, 144, 146, 148,149, 153-155, 158, 159, 294, 299, 300, 303, 308, 309, 311, 312, 317-321, 324, 325, 330-334, 337-340, and 354-357, and optionally one or more peptides being characterized by the amino acid sequences set forth in SEQ ID NO: 46, 47, 58, 61, 71, 76, 89, 105-109, 111, 113, 114, 126, 128, 130, 139, 141, 147, 151, 152, 160-167, and 265 and instructions for its use.

In one aspect, the disclosure relates to a kit for detecting a cell-mediated immune response to SARS-CoV-2 in a subject, said kit comprising one, five, ten, fifteen, twenty, twenty-five, thirty or more peptides, the peptides being selected from the group consisting of peptides being characterized by the amino acid sequences set forth in SEQ ID NO: 170, 175, 176, 178, 179, 181, 190, and 342-350, and optionally one or more peptides being characterized by the amino acid sequences set forth in SEQ ID NO: 168, 171 and 173 and instructions for its use.

In one aspect, the disclosure relates to a kit for detecting a cell-mediated immune response to SARS-CoV-2 in a subject, said kit comprising one, five, ten, fifteen, twenty, twenty-five, thirty or more peptides, the peptides being selected from the group consisting of peptides being characterized by the amino acid sequences set forth in SEQ ID NO: 235-248, 250, 252-255, 257-261, 268, 278-281, 290, 291, 351-353 and 358, and optionally one or more peptides comprising the amino acid sequences set forth in SEQ ID NO: 251, 266, 272, 274, 275, 277, 282 and 283 and instructions for its use.

In one aspect, the disclosure relates to a kit for detecting a cell-mediated immune response to SARS-CoV-2 in a subject, said kit comprising one, five, ten, fifteen, twenty, twenty-five, thirty or more peptides, the peptides being selected from the group consisting of peptides being characterized by the amino acid sequences set forth in SEQ ID NO: 293, 295-298, 301, 302, 304-306, 313-316, 322, 328, 329 and instructions for its use.

Any of the peptides and their combinations disclosed in the present disclosure may be used in the kits disclosed herein.

In some embodiments, the kit comprises a first and a second container, wherein the first container comprises one or more peptides as described herein, and the second container comprises a solution or solvent capable of dissolving the one or more peptides In some embodiments, the solvent comprised within the second container is selected from the group consisting of glycerol, water for injection, a phosphate buffered saline, a sodium phosphate buffer, a Tris buffer, a borate buffer, a succinate buffer, a histidine buffer, a citrate buffer, a potassium phosphate buffer and a mannitol solution. In some embodiments, the solution further comprises at least one preservative. In some embodiments, the preservative is selected from the group consisting of phenol, m-cresol, thiomersal, 2-phenoxyethanol and 8-hydroxy quinoline. In some embodiments, the solution further comprises at least one stabilizer. In some embodiments, the stabilizer is a non-ionic surfactant. In some embodiments, the non-ionic surfactant is Polysorbate 80, Polysorbate 20, Triton™ X-100, polyoxyethylene sorbitan, fatty acid esters, Poloxamer, polyoxyl-40-stearate and other polyoxyethylene stearates, glycerol monostearate, macrogol-8-stearat, macrogol cetostearylether 20, polyoxyethylene alkyl ethers, sorbitan monostearate and other sorbitan monoesters, polyoxyethylene castor oil derivatives, sodium lauryl sulfate, cetylpyridinium chloride or the like.

In some embodiments, the first container comprises the one or more peptides as described herein as a lyophilized dry powder. In some embodiments, the kit of the disclosure allows for the dissolution of the lyophilized peptides described herein immediately prior to the administration of the one or more peptides to a subject.

In some embodiments, the kit of the present disclosure includes devices, reagents, further containers or other components. In some embodiments, a kit of the present disclosure requires the use of an apparatus, instrument or device, including a computer.

In some embodiments, the kit of the disclosure comprises an applicator to administer the one or more peptides. In some embodiments, the applicator is a syringe, a microneedle patch, a lancet or the like.

In some embodiments, the kit of the disclosure comprises one or more peptides, wherein the one or more peptides comprise the peptides of one or more of Pool 1, Pool 2, and Pool 3. In some embodiments, the kit of the disclosure comprises one or more peptides, wherein the one or more peptides comprise the peptides of one or more of Pool 4, Pool 5, and Pool 6. In some embodiments, the kit of the disclosure comprises one or more peptides, wherein the one or more peptides comprise the peptides of one or more of Pool 7, Pool 8, and Pool 9. In some embodiments, the kit of the disclosure comprises one or more peptides, wherein the one or more peptides comprise the peptides of one or more of Pool 4, Pool 5, Pool 6, and Pool 10. In some embodiments, the kit of the disclosure comprises one or more peptides, wherein the one or more peptides comprise the peptides of one or more of Pool 4, Pool 5, Pool 6, and Pool 11. In some embodiments, the kit of the disclosure comprises one or more peptides, wherein the one or more peptides comprise the peptides of one or more of Pool 4, Pool 5, Pool 6, Pool 10, and Pool 11. In some embodiments, the kit of the disclosure comprises one or more peptides, wherein the one or more peptides comprise the peptides of one or more of Pool 7, Pool 8, Pool 9, and Pool 10. In some embodiments, the kit of the disclosure comprises one or more peptides, wherein the one or more peptides comprise the peptides of one or more of Pool 7, Pool 8, Pool 9, and Pool 11. In some embodiments, the kit of the disclosure comprises one or more peptides, wherein the one or more peptides comprise the peptides of one or more of Pool 7, Pool 8, Pool 9, Pool 10 and Pool 11.

### Combinations or Pools of the Disclosure

In another aspect of the disclosure, a combination or Pool of peptides consists of peptides characterized by the peptides of SEQ ID NO: 1-10, 12-25, 27, 28, 30-39, 41, 43 and 44.

In another aspect of the disclosure, a combination or pool of peptides consists of the peptides of SEQ ID NO: 47, 50-55, 59, 60, 62-64, 66-70, 72, 74-77, 79-81, 83, 84, 86, 87, 90-94, 96, 97, 99-101, 103, 110, 114-126, 129, 131-134, 135-138, 140, 144-146, 148,149, 153-167, 294, 299, 300, 303, 307, 308, 310-312, 317, 319, 320, 324, 325, 327, 333, 336 and 337, and optionally one or more peptides comprising the amino acid sequences set forth in SEQ ID NO: 235-282.

In another aspect of the disclosure, a combination or pool of peptides consisting of the peptides of SEQ ID NO: 174-175, 178, 179, 181, 190 and 345-350, and optionally one or more peptides comprising the amino acid sequences set forth SEQ ID NO: 284-289.

In another aspect of the disclosure, a combination or Pool of peptides consists of peptides characterized by the peptides of SEQ ID NO: 1-14, 16-45 and 292.

In another aspect of the disclosure, a combination or Pool of peptides consists of peptides characterized by the peptides of SEQ ID NO: 1-10, 12-14, 17, 18, 20-25, 27, 28, 30-39, 41, 43, 44 and 292, and optionally one or more peptides comprising the amino acid sequences set forth in SEQ ID NO: 11, 16, 19, 26, 29, 40, 42, and 45.

In another aspect of the disclosure, a combination or Pool of peptides consists of peptides characterized by the peptides of SEQ ID NO: 46-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-167, 265, 294, 299, 300, 303, 308, 309, 311, 312, 317-321, 324, 325, 330-334, 337-340, and 354-357.

In another aspect of the disclosure, a combination or pool of peptides consists of the peptides of SEQ ID NO: 47, 48, 50-55, 59, 60, 62-64, 66-70, 72, 74, 75, 77, 79-81, 83, 84, 86, 87, 90-94, 96, 99-101, 103, 110, 115-125, 129, 131-133, 136-138, 140, 142, 144, 146, 148,149, 153-155, 158, 159, 294, 299, 300, 303, 308, 309, 311, 312, 317-321, 324, 325, 330-334, 337-340, and 354-357, and optionally one or more peptides comprising the amino acid sequences set forth in SEQ ID NO: 46, 47, 58, 61, 71, 76, 89, 105-109, 111, 113, 114, 126, 128, 130, 139, 141, 147, 151, 152, 160-167, and 265.

In another aspect of the disclosure, a combination or Pool of peptides consists of peptides characterized by the peptides of SEQ ID NO: 168, 170, 171, 173, 175, 176, 178, 179, 181, 190, and 342-350.

In another aspect of the disclosure, a combination or pool of peptides consists of the peptides of SEQ ID NO: 170, 175, 176, 178, 179, 181, 190, and 342-350, and optionally one or more peptides comprising the amino acid sequences set forth in SEQ ID NO: 168, 171, and 173.

In another aspect of the disclosure, a combination or Pool of peptides consists of peptides characterized by the peptides of SEQ ID NO: 235-248, 250-255, 257-261, 266, 268, 272, 274, 275, 277-283, 290, 291, 351-353 and 358.

In another aspect of the disclosure, a combination or pool of peptides consists of the peptides of SEQ ID NO: 235-248, 250, 252-255, 257-261, 268, 278-281, 290, 291, 351-353 and 358, and optionally one or more peptides comprising the amino acid sequences set forth in SEQ ID NO: 251, 266, 272, 274, 275, 277, 282 and 283.

In another aspect of the disclosure, a combination or Pool of peptides consists of peptides characterized by the peptides of SEQ ID NO: 293, 295-298, 301, 302, 304-306, 313-316, 322, 328 and 329.

### EXAMPLES

### Example 1. Generation of the Peptides of the Disclosure

The peptides of the disclosure are manufactured through solid-phase peptide synthesis (SPPS) in accordance with a general method as described in Lloyd-Williams P. et al. (1997) Chemical approaches to the synthesis of peptides and proteins. CRC Press. Boca Raton. The solid support consists of small, polymeric resin beads functionalized with reactive groups that link to the nascent peptide chain. The protection of the N-terminal and side chains is performed using the Boc/Bzl or Fmoc/tBu SPPS.

The peptides are manufactured as Trifluoroacetate (TFA) salts. The residual TFA present in the peptides is removed before using any of the peptides in the methods of the disclosure.

The peptides of the disclosure are purified through Ultra Performance Liquid Chromatography (UPLC). This step separates the peptides from impurities from the synthesis steps, such as isomers, deletion sequences, peptide products from side reactions with free coupling and protecting groups or peptides that have undergone side-chain reactions. The peptides purity is measured as a percentage of the peptide to impurities that absorb at the peptide bond absorption wavelength (210-220 nm). The peptides purity obtained is preferably higher than 85%.

Lyophilized peptides are extracted with a solution of NaCl, NaHCO₃ and glycerol. Prior to the administration in the skin, the peptides are prepared in a solution comprising Polysorbate 80 and phenol in a sterile isotonic phosphate buffered saline.

### Example 2. Skin Test to Detect a Cell-Mediated Immune Response Against SARS-CoV-2

The skin test preparation consists of 3 pools of peptides: (1) peptides of SEQ ID NO: 1-45; (2) peptides of SEQ ID NO: 46-167 and (3) peptides of SEQ ID NO: 168-183. The peptides are tested in a group of subjects by intradermally injecting the pools of peptides and a negative control (only solvent) in the ventral surface of the forearm, about 1 mm to about 2 mm within the dermis. 1 μg of the pools of peptides is injected intradermally in a 0.1 ml solution.

The skin reaction is monitored about 48 to 72 hours post-injection by measuring the longest and midpoint orthogonal diameters of the indurated area in millimeters. The mean of the longest and midpoint orthogonal diameters of the indurated area is reported as the DTH response. Reactions are considered positive when the induration is about >10 mm in diameter about 48 hours after injection. Reactions are also further monitored at about 72 hours to 96 hours.

A positive immune reaction in a subject is indicative of the subject having developed a cell-mediated immune response to SARS-CoV-2 and, therefore, has developed cell-mediated immunity against SARS-CoV-2 or has an active SARS-CoV-2 infection. The skin tests may be also correlated with a serological immune response and any symptoms present in the subject.

### Example 3. Skin Test to Determine if a Potential or Approved Vaccine Against SARS-CoV-2 Elicits a Cell-Mediated Immune Response

Subjects are vaccinated with a candidate or approved vaccine against SARS-CoV-2. The peptides of the disclosure are further administered to those subjects to determine if the vaccine elicits a cell-mediated immune response.

The skin test preparation consists of 3 pools of peptides: (1) peptides of SEQ ID NO: 1-45; (2) peptides of SEQ ID NO: 46-167 and (3) peptides of SEQ ID NO: 168-183. The peptides are tested in a group of subjects by intradermally injecting the pools of peptides and a negative control (only solvent) in the ventral surface of the forearm, about 1 mm to about 2 mm within the dermis. 1 μg of the pools of peptides is injected intradermally in a 0.1 ml solution.

The skin reaction is monitored about 48 to 72 hours post-injection by measuring the longest and midpoint orthogonal diameters of the indurated area in millimeters. The mean of the longest and midpoint orthogonal diameters of the indurated area is reported as the DTH response. Reactions are considered positive when the induration is about >10 mm in diameter about 48 hours after injection. Reactions are also further monitored at about 72 hours to 96 hours.

A positive immune reaction in a subject is indicative of the vaccine having elicited a cell-mediated immune response in the subject. The skin tests may also be correlated with another assay that determines the clinical benefit of the vaccine, such as Enzyme-Linked Immunosorbent spot (ELISpot) assay, which is commonly used to measure antigen-specific T cells in humans.

### Example 4. Skin Test Using the Methods of this Disclosure

125 subjects are separated into 5 cohorts. Cohort 1 includes 25 healthy uninfected or unexposed subjects (“naïve”). Cohort 2 includes 25 subjects with acute SARS-CoV-2 infection or symptomatic SARS-CoV-2 infection (“active infection”). Cohort 3 includes 25 subjects who test positive via, for example, real-time polymerase chain reaction for SARS CoV-2 viral RNA but are asymptomatic or with mild symptoms of SARS-CoV-2 infection (“shedders”). Cohort 4 includes 25 subjects who recovered from SARS-CoV-2 infection and are at least 2 months past recovery of SARS-CoV-2 infection (“convalescent”). Cohort 5 includes 25 subjects with SARS-CoV-2 antigen exposure, but no history of SARS-CoV-2 infection (“spike-immune”). Cohort 5 may include (but is not limited to) subjects participating in SARS-CoV-2 spike vaccine trials.

The study consists of a pre-screening check, an administration visit, a follow-up visit and an End of Study (EoS) follow-up by telephone. The duration of the study from screening to the end of the study is approximately 4 weeks. Subjects satisfying all inclusion/exclusion criteria at Visit 1 undergo a nasopharyngeal (NP) swab and blood samples are collected for peripheral blood mononuclear cell (PBMC) assays and routine clinical laboratory tests.

Each subject is intradermally injected with a 0.1 mL dose of one or more of the following:

1) Peptide Pool 1: SEQ ID NO: 1-10, 12-25, 27, 28, 30-39, 41, 43 and 44;

2) Peptide Pool 2: SEQ ID NO: 47, 50-55, 59, 60, 62-64, 66-70, 72, 74-77, 79-81, 83, 84, 86, 87, 90-94, 96, 97, 99-101, 103, 110, 114-126, 129, 131-134, 135-138, 140, 144-146, 148, 149, 153-167, 294, 299, 300, 303, 308, 310, 311, 312, 317, 319, 320, 324, 325, 237, 333, 336 and 337, and optionally one or more peptides of SEQ ID NO: 235-283;

3) Peptide Pool 3: SEQ ID NO: 170, 172, 174-176, 178, 179, 181, 190, 341, 342 and 345-350, and optionally one or more peptides of SEQ ID NO: 284-289;

4) Vehicle control (negative control); and

5) Commercially available Candida albicans antigens (CANDIN®) (positive control).

The peptides are administered in a dose-escalating manner starting with a concentration of 0.05 μg peptide per 100 μL (dose strength “1×”) in phosphate buffered saline. If no adverse reaction is observed in the subjects after at least 1 hour, peptides at a concentration of 0.5 peptide per 100 μL (dose strength “10×”) are subsequently administered. The subjects are injected at pre-determined sites two inches apart from each other on the volar aspect of one forearm with a 0.1 mL 1× dose of each of the peptide pools, followed by the administration of a 0.1 mL 10× dose of each of the peptide pools, vehicle control (negative control), and commercially available Candida albicans antigens (CANDIN®) (positive control) at pre-determined sites on the volar aspect of the other forearm. Each subject will receive a total of 8 intradermal injections at least 2 inches apart.

During the Baseline/Screening Visit (Day 1, Visit 1) and prior to the scheduled injection administration, a general health inquiry (and a physical examination, if appropriate), adverse events (AEs), concomitant medications and pregnancy status is checked, and vital signs are taken. After the injection, subjects remain in the clinic for observation of potential injection site reactions or AEs for 60 minutes, and subsequently are able to leave if no safety concerns have been detected.

Delayed type hypersensitivity reactions are assessed after 48, 78 and 96 hours after administration.

Assessment of Anti-SARS-CoV-2 antibody titers are performed by enzyme-linked immunosorbent assay (ELISA). Flow cytometry is performed on PBMCs. For in vitro lymphocyte stimulation, the 3 test peptide mixtures are used along with commercially available benign (common cold) Coronavirus-specific peptides.

Skin test results are correlated with clinical history and laboratory findings and establish:

1. That the skin test identifies cell mediated immune responses to SARS-CoV-2.
2. The sensitivity and specificity of the test relative to clinical history and laboratory findings in acute, subacute, and convalescent subjects.

Subjects grade local and systemic skins reactions using a 7-point Likert Scale (Table 2). Adverse events categorized as systemic indicate whether 1) the adverse event does not interfere with the subject's routine activities, symptoms do not require therapy or medical evaluation and signs and symptoms are transient; 2) the adverse event interferes with the subject's daily routine but are usually improved by simple therapeutic measures, and usual routine activities can still be carried out; and 3) the adverse event results in the inability to perform routine activities and generally require systemic drug therapy or other treatment.

TABLE 2
7-point Likert Scale for grading local skin reaction

| | | Min- | Very | | Mod- | | Very |
|---|---|---|---|---|---|---|---|
| | Absent | imal | Mild | Mild | erate | Severe | Severe |
| | 0 | 1 | 2 | 3 | 4 | 5 | 6 |
| Injection | | | | | | | |
| Site Adverse | | | | | | | |
| Experiences | | | | | | | |
| Systemic | | | | | | | |
| Adverse | | | | | | | |
| Experiences | | | | | | | |

Primary Efficacy Endpoints of the study include area of induration at injection sites on the volar forearm. Secondary Efficacy Endpoints of the study include: 1. Anti-SARS-CoV-2 antibody titer (ELISA); and 2. Flow cytometry of PBMCs (e.g. SARS-CoV-2-specific T-cell responses are measured by stimulating PBMC in vitro with test peptides. A Th1 response is characterized by CD4+ T cells expressing IFN-γ and/or IL-2 and not IL-4, IL-5 and/or IL13; a Th2 response is measured by CD4+ T cells expressing IL-4, IL-5 and/or IL-13 and CD40L by 142 CD4+ T cells; a Th17 response is measured by CD4+ T cells expressing IL-17. SARS-CoV-2-specific CD8+ T cell responses is measured by the expression of IFN-γ and/or IL-2 cytokines). Optionally another pharmaceutically acceptable positive control can be used during flow cytometry analyes, e.g., a tetanus antigen).

Subjects will be monitored for safety for at least 6 months after the last investigational product exposure to include assessments of serious and other medically attended adverse events, and specifically COVID-19 diseases. Safety follow-up times include e.g. 1 month and 6 months, which may be conducted by telephone and/or in-person visits.

### Example 5. Manufacturing Process

All investigational products are manufactured under current Good Manufacturing Practices (cGMP) by solid phase peptide synthesis (SPPS), employing Fmoc-amino acid chemistry. SPPS is a repetitive procedure, during which a peptide is assembled from the C-terminus to the N-terminus on a suitable resin (e.g. pre-loaded Wang polystyrene resin or Cl TCP (Cl) ProTide resin) as a solid support. In a first step, the C terminal Fmoc amino acid is coupled to the resin (only for Cys) all other syntheses are performed on pre-loaded Wang resins. Following deprotection of the Fmoc-protecting group by piperidine in dimethylformamide and subsequent washing to remove remaining piperidine, the next Fmoc amino acid is coupled to the resin using an activation reagent (e.g. diisopropyl carbodiimide) in the presence of ethyl (hydroximino) cyanoacetate (Oxyma). The process is continued using the subsequent sequence specific protected amino acids until the entire sequence has been built up. Side chain groups of the amino acid derivates are protected by acid labile protecting groups. The Fmoc cleavage and coupling steps are conducted on a Liberty PRIME peptide synthesizer from CEM. All coupling and deprotections are performed at elevated temperature with microwave radiation. Deprotection is conducted at about 110° C., coupling at approximately 105° C.

Upon completion, the resin-bound peptides are washed and dried. The individual peptides are cleaved from the resin by treatment of the resins with trifluoroacetic acid and scavengers. During this cleavage step, all remaining protecting groups are removed. Scavengers are added to prevent the reaction of reactive species formed during cleavage with the crude peptide. The crude peptide, which is now a trifluoroacetate salt, is subsequently precipitated using ethyl ether and dried in a vacuum oven.

Purification of the crude peptides is conducted by RP-HPLC using trifluoroacetic acid additive in the mobile phase (acetonitrile/water). Peptide fractions are collected and freeze dried.

### Example 6. Skin Test Using the Methods of this Disclosure

90 subjects (or 150 subjects) are separated into 3 cohorts. Cohort 1 includes 30 (or 50) healthy uninfected or unexposed subjects. Cohort 2 includes 30 subjects (or 50 subjects) are confirmed to have recovered for at least two months from SARS-CoV-2 infection. Cohort 3 includes 30 subjects (or 50 subjects) who received a complete SARS-CoV-2 vaccine course for at least 4 weeks.

The study consists of a pre-screening check (e.g., Visit 1; Days 1-14), a baseline/screening visit (e.g., Visit 1, Day 1), and follow-up assessments (e.g., Visit 2, Day2; Visit 3, Day 3; Visit 4, Day 4; Visit 5, Day 5; visit 6, Day 30; Visit 7, Day 180) to monitor safety and to evaluate the presence or absence of delayed-type hypersensitivity (DTH) reaction in response to intradermal injections of peptide pools and control groups (see, e.g., Paragraph [0311]). Subjects satisfying all inclusion/exclusion criteria at Visit 1 undergo a nasopharyngeal (NP) swab for rapid detection of SARS-CoV-2. If the subject is negative for SARS-CoV-2, the subject will proceed with blood draws follow by intradermal injection of the experimental peptides and controls in two stages.

In Stage 1, each subject is intradermally injected with 0.1 mL of a diluted dose (e.g., 1:10 dilution) with a concentration of 0.025 μg peptide per 100 μL of one or more of the following in the left forearm:

1) Peptide Pool 4: SEQ ID NO: 1-14, 16-45, and 292;

2) Peptide Pool 5: SEQ ID NO: 46-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-167, 265, 294, 299, 300, 303, 308, 309, 311, 312, 317-321, 324, 325, 330-334, 337-340, and 354-357;

3) Peptide Pool 6: SEQ ID NO: 168, 170, 171, 173, 175, 176, 178, 179, 181, 190, and 342-350; and

4) Vehicle control (negative control, also referred to as diluent) of 0.02 M phosphate with 0.31% polysorbate 20 and 4.6% mannitol, pH 7.0.

Each subject is also intradermally injected with a 0.1 mL dose of commercially available Candida albicans antigens (CANDIN®) (positive control) in the right forearm.

In Stage 2, each subject is intradermally injected with a 0.1 mL undiluted dose with a concentration of 0.25 μg peptide per 100 μL of one or more of the following in the right arm:

1) Peptide Pool 4: SEQ ID NO: 1-14, 16-45 and 292;

2) Peptide Pool 5: SEQ ID NO: 46-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-167, 265, 294, 299, 300, 303, 308, 309, 311, 312, 317-321, 324, 325, 330-334, 337-340, and 354-357; and

3) Peptide Pool 6: SEQ ID NO: 168, 170, 171, 173, 175, 176, 178, 179, 181, 190, and 342-350.

After Stage 1 administration, subjects are monitored for local site reactions and systemic adverse reactions for 60 minutes. At 30 minutes post administration, vital signs (e.g., systolic and diastolic blood pressure, heart rate, and temperature) are measured. If no systemic adverse reactions or unusual local site reactions are observed, the subjects will proceed to Stage 2 administration.

After Stage 2 administration, subjects will be monitored over the course of 30 minutes for unusual local site reactions and systemic adverse reactions. At 30 minutes post-administration, vital signs are assessed. If no evidence of systemic adverse reactions or unusual local site reactions occur, subjects are free to leave the clinic and are contacted 24 hours later for a brief telephone or in-person safety follow-up visit (e.g., Visit 2, Day 2). Subjects are instructed to keep the injection sites clean, uncovered, and to not scratch or rub the area. Subjects are asked to return to the clinic for an in-person assessment of the skin test result at various time points, starting with 48 hours post kin test administration (e.g., Visit 3, Day 3), followed by two subsequent visits at 72 hours (e.g., Visit 4, Day 4) and 96 hours (e.g., Visit 5, Day 5) post skin test administration. Subjects have two telephone safety follow-up visits (e.g., Visit 6, Day 30 and Visit 7, Day 180). The duration of the study from pre-screening contact to the last safety monitoring is approximately 6 months.

Assessment of anti-SARS-CoV-2 antibody titers are performed for the detection and quantification of antibodies to SARS-CoV-2 (for e.g., by enzyme-linked immunosorbent assay (ELISA)). Flow cytometric analysis of SARS-CoV-2-specific T-cell responses are measured, along with in vitro positive controls (e.g., benign (common cold) Coronavirus-specific peptides). To characterize Th17 response, IL-17 is evaluated using an enzyme-linked immune absorbent spot assay (ELISpot). A T-MAP assay is performed to aid the identification of individuals with an adaptive T-cell immune response to SARS-CoV-2, which indicates recent or prior infection with SARS-CoV-2.

Additional in vitro positive controls that are tested include Candida albican peptides and a PepMix CEFX Ultra SuperStim Pool (PM-CEFX) containing tetanus, herpes simplex virus, human papillomavirus, measles, mumps, rubella, and polio peptides, are analyzed to confirm that subjects have intact T-cell immunity and are no immunodeficient.

Primary Efficacy Endpoint of the study includes maximal area of induration (e.g., >5 mm) at injection sites on the volar forearms at 48 hours, 72 hours and 96 hours post skin test administration. Secondary Efficacy Endpoints of the study include: 1) Correlation of the presence or absence of DTH reactions with clinical history to estimate sensitivity and specificity of the peptide pools as a marker of active or recovered infection relative to clinical history of infection; 2) Correlation of DTH reaction to adaptive T-cell immune response to SARS-CoV-2 assessed by immunoSEQ® T-MAP™ COVID; 3) Characterization of Th1 and Th2 responses to DTH reaction by measuring cytokine production and cell surface T-cell phenotype markers by intracellular cytokine staining; and 4) Characterization of Th17 response to DTH reaction by ELISpot. Exploratory efficacy endpoints include: 1) Correlation of DTH reaction with in vitro assays of immune response including anti-SARS-CoV-2 levels; 2) Correlation of DTH reaction with flow cytometry of PBMCs (e.g., SARS-CoV-2-specific T-cell responses); and 3) Comparison of DTH reactions in recovered subjects from SARS-CoV-2 infection (e.g., Cohort 2) with COVID-19 vaccine recipients (e.g., Cohort 3).

### Example 7. Skin Test Using the Methods of this Disclosure

90 subjects (or 150 subjects) are separated into 3 cohorts. Cohort 1 includes 30 (or 50) healthy uninfected or unexposed subjects. Cohort 2 includes 30 subjects (or 50 subjects) are confirmed to have recovered for at least two months from SARS-CoV-2 infection. Cohort 3 includes 30 subjects (or 50 subjects) who received a complete SARS-CoV-2 vaccine course for at least 4 weeks.

The study consists of a pre-screening check (e.g., Visit 1; Days 1-14), a baseline/screening visit (e.g., Visit 1, Day 1), and follow-up assessments (e.g., Visit 2, Day2; Visit 3, Day 3; Visit 4, Day 4; Visit 5, Day 5; visit 6, Day 30; Visit 7, Day 180) to monitor safety and to evaluate the presence or absence of delayed-type hypersensitivity (DTH) reaction in response to intradermal injections of peptide pools and control groups (see, e.g., Paragraph [0320]). Subjects satisfying all inclusion/exclusion criteria at Visit 1 undergo a nasopharyngeal (NP) swab for rapid detection of SARS-CoV-2. If the subject is negative for SARS-CoV-2, the subject will proceed with blood draws follow by intradermal injection of the experimental peptides and controls in two stages.

In Stage 1, each subject is intradermally injected with 0.1 mL of a diluted dose (e.g., 1:10 dilution) with a concentration of 0.025 μg peptide per 100 μL of one or more of the following in the left forearm:

1) Peptide Pool 7: SEQ ID NO: 1-10, 12-14, 17, 18, 20-25, 27, 28, 30-39, 41, 43, 44 and 292 and optionally one or more peptides of SEQ ID NO: 11, 16, 19, 26, 29, 40, 42 and 45;

2) Peptide Pool 8: SEQ ID NO: 47, 48, 50-55, 59, 60, 62-64, 66-70, 72, 74, 75, 77, 79-81, 83, 84, 86, 87, 90-94, 96, 99-101, 103, 110, 115-125, 129, 131-133, 136-138, 140, 142, 144, 146, 148,149, 153-155, 158, 159, 294, 299, 300, 303, 308, 309, 311, 312, 317-321, 324, 325, 330-334, 337-340, and 354-357, and optionally one or more peptides of SEQ ID NO: 46, 47, 58, 61, 71, 76, 89, 105-109, 111, 113, 114, 126, 128, 130, 139, 141, 147, 151, 152, 160-167, and 265;

3) Peptide Pool 9: SEQ ID NO: 170, 175, 176, 178, 179, 181, 190, and 342-350, and optionally one or more peptides of SEQ ID NO: 168, 171, and 173; and

4) Vehicle control (negative control, also referred to as diluent) of 0.02 M phosphate with 0.31% polysorbate 20 and 4.6% mannitol, pH 7.0.

Each subject is also intradermally injected with a 0.1 mL dose of commercially available Candida albicans antigens (CANDIN®) (positive control) in the right forearm.

In Stage 2, each subject is intradermally injected with a 0.1 mL undiluted dose with a concentration of 0.25 μg peptide per 100 μL of one or more of the following in the right arm:

1) Peptide Pool 7: SEQ ID NO: 1-10, 12-14, 17, 18, 20-25, 27, 28, 30-39, 41, 43, 44 and 292, and optionally one or more peptides of SEQ ID NO: 11, 16, 19, 26, 29, 40, 42 and 45;

2) Peptide Pool 8: SEQ ID NO: 47, 48, 50-55, 59, 60, 62-64, 66-70, 72, 74, 75, 77, 79-81, 83, 84, 86, 87, 90-94, 96, 99-101, 103, 110, 115-125, 129, 131-133, 136-138, 140, 142, 144, 146, 148,149, 153-155, 158, 159, 294, 299, 300, 303, 308, 309, 311, 312, 317-321, 324, 325, 330-334, 337-340, and 354-357, and optionally one or more peptides of SEQ ID NO: 46, 47, 58, 61, 71, 76, 89, 105-109, 111, 113, 114, 126, 128, 130, 139, 141, 147, 151, 152, 160-167, and 265;

3) Peptide Pool 9: SEQ ID NO: 170, 175, 176, 178, 179, 181, 190, and 342-350, and optionally one or more peptides of SEQ ID NO: 168, 171, and 173;

After Stage 1 administration, subjects are monitored for local site reactions and systemic adverse reactions for 60 minutes. At 30 minutes post administration, vital signs (e.g., systolic and diastolic blood pressure, heart rate, and temperature) are measured. If no systemic adverse reactions or unusual local site reactions are observed, the subjects will proceed to Stage 2 administration.

After Stage 2 administration, subjects will be monitored over the course of 30 minutes for unusual local site reactions and systemic adverse reactions. At 30 minutes post-administration, vital signs are assessed. If no evidence of systemic adverse reactions or unusual local site reactions occur, subjects are free to leave the clinic and are contacted 24 hours later for a brief telephone or in-person safety follow-up visit (e.g., Visit 2, Day 2). Subjects are instructed to keep the injection sites clean, uncovered, and to not scratch or rub the area. Subjects are asked to return to the clinic for an in-person assessment of the skin test result at various time points, starting with 48 hours post kin test administration (e.g., Visit 3, Day 3), followed by two subsequent visits at 72 hours (e.g., Visit 4, Day 4) and 96 hours (e.g., Visit 5, Day 5) post skin test administration. Subjects have two telephone safety follow-up visits (e.g., Visit 6, Day 30 and Visit 7, Day 180). The duration of the study from pre-screening contact to the last safety monitoring is approximately 6 months.

Assessment of anti-SARS-CoV-2 antibody titers are performed for the detection and quantification of antibodies to SARS-CoV-2 (for e.g., by enzyme-linked immunosorbent assay (ELISA)). Flow cytometric analysis of SARS-CoV-2-specific T-cell responses are measured, along with in vitro positive controls (e.g., benign (common cold) Coronavirus-specific peptides). To characterize Th17 response, IL-17 is evaluated using an enzyme-linked immune absorbent spot assay (ELISpot). A T-MAP assay is performed to aid the identification of individuals with an adaptive T-cell immune response to SARS-CoV-2, which indicates recent or prior infection with SARS-CoV-2.

Additional in vitro positive controls that are tested include Candida albican peptides and a PepMix CEFX Ultra SuperStim Pool (PM-CEFX) containing tetanus, herpes simplex virus, human papillomavirus, measles, mumps, rubella, and polio peptides, are analyzed to confirm that subjects have intact T-cell immunity and are no immunodeficient.

Primary Efficacy Endpoint of the study includes maximal area of induration (e.g., >5 mm) at injection sites on the volar forearms at 48 hours, 72 hours and 96 hours post skin test administration. Secondary Efficacy Endpoints of the study include: 1) Correlation of the presence or absence of DTH reactions with clinical history to estimate sensitivity and specificity of the peptide pools as a marker of active or recovered infection relative to clinical history of infection; 2) Correlation of DTH reaction to adaptive T-cell immune response to SARS-CoV-2 assessed by immunoSEQ® T-MAP′ COVID; 3) Characterization of Th1 and Th2 responses to DTH reaction by measuring cytokine production and cell surface T-cell phenotype markers by intracellular cytokine staining; and 4) Characterization of Th17 response to DTH reaction by ELISpot. Exploratory efficacy endpoints include: 1) Correlation of DTH reaction with in vitro assays of immune response including anti-SARS-CoV-2 levels; 2) Correlation of DTH reaction with flow cytometry of PBMCs (e.g., SARS-CoV-2-specific T-cell responses); and 3) Comparison of DTH reactions in recovered subjects from SARS-CoV-2 infection (e.g., Cohort 2) with COVID-19 vaccine recipients (e.g., Cohort 3).

### Example 8. Skin Test Using the Methods of this Disclosure

90 subjects (or 150 subjects) are separated into 3 cohorts. Cohort 1 includes 30 (or 50) healthy uninfected or unexposed subjects. Cohort 2 includes 30 subjects (or 50 subjects) are confirmed to have recovered for at least two months from SARS-CoV-2 infection. Cohort 3 includes 30 subjects (or 50 subjects) who received a complete SARS-CoV-2 vaccine course for at least 4 weeks.

The study consists of a pre-screening check (e.g., Visit 1; Days 1-14), a baseline/screening visit (e.g., Visit 1, Day 1), and follow-up assessments (e.g., Visit 2, Day2; Visit 3, Day 3; Visit 4, Day 4; Visit 5, Day 5; visit 6, Day 30; Visit 7, Day 180) to monitor safety and to evaluate the presence or absence of delayed-type hypersensitivity (DTH) reaction in response to intradermal injections of peptide pools and control groups (see, e.g., Paragraph [0329]). Subjects satisfying all inclusion/exclusion criteria at Visit 1 undergo a nasopharyngeal (NP) swab for rapid detection of SARS-CoV-2. If the subject is negative for SARS-CoV-2, the subject will proceed with blood draws follow by intradermal injection of the experimental peptides and controls in two stages.

In Stage 1, each subject is intradermally injected with 0.1 mL of a diluted dose (e.g., 1:10 dilution) with a concentration of 0.025 μg peptide per 100 μL of one or more of the following in the left forearm:

1) Peptide Pool 4: SEQ ID NO: 1-14, 16-45 and 292;

2) Peptide Pool 5: SEQ ID NO: 46-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-167, 265, 294, 299, 300, 303, 308, 309, 311, 312, 317-321, 324, 325, 330-334, 337-340, and 354-357;

3) Peptide Pool 6: SEQ ID NO: 168, 170, 171, 173, 175, 176, 178, 179, 181, 190, and 342-350;

4) Peptide Pool 10: SEQ ID NO: 235-248, 250, 252-255, 257-261, 268, 278-281, 290, 291, 351-353 and 358, and optionally one or more of SEQ ID NO: 251, 266, 272, 274, 275, 277, 282 and 283;

5) Peptide Pool 11: SEQ ID NO: 293, 295-298, 301, 302, 304-306, 313-316, 322, 328 and 329; and

6) Vehicle control (negative control, also referred to as diluent) of 0.02 M phosphate with 0.31% polysorbate 20 and 4.6% mannitol, pH 7.0.

Each subject is also intradermally injected with a 0.1 mL dose of commercially available Candida albicans antigens (CANDIN®) (positive control) in the right forearm.

In Stage 2, each subject is intradermally injected with a 0.1 mL undiluted dose with a concentration of 0.25 μg peptide per 100 μL of one or more of the following in the right arm:

1) Peptide Pool 4: SEQ ID NO: 1-14, 16-45 and 292;

2) Peptide Pool 5: SEQ ID NO: 46-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-167, 265, 294, 299, 300, 303, 308, 309, 311, 312, 317-321, 324, 325, 330-334, 337-340, and 354-357;

3) Peptide Pool 6: SEQ ID NO: 168, 170, 171, 173, 175, 176, 178, 179, 181, 190, and 342-350;

4) Peptide Pool 10: SEQ ID NO: 235-248, 250, 252-255, 257-261, 268, 278-281, 290, 291, 351-353 and 358, and optionally one or more peptides of SEQ ID NO: 251, 266, 272, 274, 275, 277, 282 and 283; and

5) Peptide Pool 11: SEQ ID NO: 293, 295-298, 301, 302, 304-306, 313-316, 322, 328 and 329.

After Stage 1 administration, subjects are monitored for local site reactions and systemic adverse reactions for 60 minutes. At 30 minutes post administration, vital signs (e.g., systolic and diastolic blood pressure, heart rate, and temperature) are measured. If no systemic adverse reactions or unusual local site reactions are observed, the subjects will proceed to Stage 2 administration.

After Stage 2 administration, subjects will be monitored over the course of 30 minutes for unusual local site reactions and systemic adverse reactions. At 30 minutes post-administration, vital signs are assessed. If no evidence of systemic adverse reactions or unusual local site reactions occur, subjects are free to leave the clinic and are contacted 24 hours later for a brief telephone or in-person safety follow-up visit (e.g., Visit 2, Day 2). Subjects are instructed to keep the injection sites clean, uncovered, and to not scratch or rub the area. Subjects are asked to return to the clinic for an in-person assessment of the skin test result at various time points, starting with 48 hours post kin test administration (e.g., Visit 3, Day 3), followed by two subsequent visits at 72 hours (e.g., Visit 4, Day 4) and 96 hours (e.g., Visit 5, Day 5) post skin test administration. Subjects have two telephone safety follow-up visits (e.g., Visit 6, Day 30 and Visit 7, Day 180). The duration of the study from pre-screening contact to the last safety monitoring is approximately 6 months.

Assessment of anti-SARS-CoV-2 antibody titers are performed for the detection and quantification of antibodies to SARS-CoV-2 (for e.g., by enzyme-linked immunosorbent assay (ELISA)). Flow cytometric analysis of SARS-CoV-2-specific T-cell responses are measured, along with in vitro positive controls (e.g., benign (common cold) Coronavirus-specific peptides). To characterize Th17 response, IL-17 is evaluated using an enzyme-linked immune absorbent spot assay (ELISpot). A T-MAP assay is performed to aid the identification of individuals with an adaptive T-cell immune response to SARS-CoV-2, which indicates recent or prior infection with SARS-CoV-2.

Additional in vitro positive controls that are tested include Candida albican peptides and a PepMix CEFX Ultra SuperStim Pool (PM-CEFX) containing tetanus, herpes simplex virus, human papillomavirus, measles, mumps, rubella, and polio peptides, are analyzed to confirm that subjects have intact T-cell immunity and are no immunodeficient.

Primary Efficacy Endpoint of the study includes maximal area of induration (e.g., >5 mm) at injection sites on the volar forearms at 48 hours, 72 hours and 96 hours post skin test administration. Secondary Efficacy Endpoints of the study include: 1) Correlation of the presence or absence of DTH reactions with clinical history to estimate sensitivity and specificity of the peptide pools as a marker of active or recovered infection relative to clinical history of infection; 2) Correlation of DTH reaction to adaptive T-cell immune response to SARS-CoV-2 assessed by immunoSEQ® T-MAP′ COVID; 3) Characterization of Th1 and Th2 responses to DTH reaction by measuring cytokine production and cell surface T-cell phenotype markers by intracellular cytokine staining; and 4) Characterization of Th17 response to DTH reaction by ELISpot. Exploratory efficacy endpoints include: 1) Correlation of DTH reaction with in vitro assays of immune response including anti-SARS-CoV-2 levels; 2) Correlation of DTH reaction with flow cytometry of PBMCs (e.g., SARS-CoV-2-specific T-cell responses); and 3) Comparison of DTH reactions in recovered subjects from SARS-CoV-2 infection (e.g., Cohort 2) with COVID-19 vaccine recipients (e.g., Cohort 3).

### Example 9. Skin Test Using the Methods of this Disclosure

90 subjects (or 150 subjects) are separated into 3 cohorts. Cohort 1 includes 30 (or 50) healthy uninfected or unexposed subjects. Cohort 2 includes 30 subjects (or 50 subjects) are confirmed to have recovered for at least two months from SARS-CoV-2 infection. Cohort 3 includes 30 subjects (or 50 subjects) who received a complete SARS-CoV-2 vaccine course for at least 4 weeks.

The study consists of a pre-screening check (e.g., Visit 1; Days 1-14), a baseline/screening visit (e.g., Visit 1, Day 1), and follow-up assessments (e.g., Visit 2, Day2; Visit 3, Day 3; Visit 4, Day 4; Visit 5, Day 5; visit 6, Day 30; Visit 7, Day 180) to monitor safety and to evaluate the presence or absence of delayed-type hypersensitivity (DTH) reaction in response to intradermal injections of peptide pools and control groups (see, e.g., Paragraph [0338]). Subjects satisfying all inclusion/exclusion criteria at Visit 1 undergo a nasopharyngeal (NP) swab for rapid detection of SARS-CoV-2. If the subject is negative for SARS-CoV-2, the subject will proceed with blood draws follow by intradermal injection of the experimental peptides and controls in two stages.

In Stage 1, each subject is intradermally injected with 0.1 mL of a diluted dose (e.g., 1:10 dilution) with a concentration of 0.025 μg peptide per 100 μL of one or more of the following in the left forearm:

1) Peptide Pool 7: SEQ ID NO: 1-10, 12-14, 17, 18, 20-25, 27, 28, 30-39, 41, 43, 44 and 292, and optionally one or more peptides of SEQ ID NO: 11, 16, 19, 26, 29, 40, 42 and 45;

2) Peptide Pool 8: SEQ ID NO: 47, 48, 50-55, 59, 60, 62-64, 66-70, 72, 74, 75, 77, 79-81, 83, 84, 86, 87, 90-94, 96, 99-101, 103, 110, 115-125, 129, 131-133, 136-138, 140, 142, 144, 146, 148,149, 153-155, 158, 159, 294, 299, 300, 303, 308, 309, 311, 312, 317-321, 324, 325, 330-334, 337-340, and 354-357, and optionally one or more peptides of SEQ ID NO: 46, 47, 58, 61, 71, 76, 89, 105-109, 111, 113, 114, 126, 128, 130, 139, 141, 147, 151, 152, 160-167, and 265;

3) Peptide Pool 9: SEQ ID NO: 170, 175, 176, 178, 179, 181, 190, and 342-350, and optionally one or more peptides of SEQ ID NO: 168, 171, and 173;

4) Peptide Pool 10: SEQ ID NO: 235-248, 250, 252-255, 257-261, 268, 278-281, 290, 291, 351-353 and 358, and optionally one or more peptides of SEQ ID NO: 251, 266, 272, 274, 275, 277, 282 and 283;

5) Peptide Pool 11: SEQ ID NO: 293, 295-298, 301, 302, 304-306, 313-316, 322, 328 and 329 and

6) Vehicle control (negative control, also referred to as diluent) of 0.02 M phosphate with 0.31% polysorbate 20 and 4.6% mannitol, pH 7.0.

Each subject is also intradermally injected with a 0.1 mL dose of commercially available Candida albicans antigens (CANDIN®) (positive control) in the right forearm.

In Stage 2, each subject is intradermally injected with a 0.1 mL undiluted dose with a concentration of 0.25 μg peptide per 100 μL of one or more of the following in the right arm:

1) Peptide Pool 7: SEQ ID NO: 1-10, 12-14, 17, 18, 20-25, 27, 28, 30-39, 41, 43, 44 and 292, and optionally one or more peptides of SEQ ID NO: 11, 16, 19, 26, 29, 40, 42 and 45;

2) Peptide Pool 8: SEQ ID NO: 47, 48, 50-55, 59, 60, 62-64, 66-70, 72, 74, 75, 77, 79-81, 83, 84, 86, 87, 90-94, 96, 99-101, 103, 110, 115-125, 129, 131-133, 136-138, 140, 142, 144, 146, 148,149, 153-155, 158, 159, 294, 299, 300, 303, 308, 309, 311, 312, 317-321, 324, 325, 330-334, 337-340, and 354-357, and optionally one or more peptides of SEQ ID NO: 46, 47, 58, 61, 71, 76, 89, 105-109, 111, 113, 114, 126, 128, 130, 139, 141, 147, 151, 152, 160-167, and 265;

3) Peptide Pool 9: SEQ ID NO: 170, 175, 176, 178, 179, 181, 190, and 342-350, and optionally one or more peptides of SEQ ID NO: 168, 171, and 173;

4) Peptide Pool 10: SEQ ID NO: 235-248, 250, 252-255, 257-261, 268, 278-281, 290, 291, 351-353 and 358, and optionally one or more peptides of SEQ ID NO: 251, 266, 272, 274, 275, 277, 282 and 283; and

5) Peptide Pool 11: SEQ ID NO: 293, 295-298, 301, 302, 304-306, 313-316, 322, 328 and 329.

After Stage 1 administration, subjects are monitored for local site reactions and systemic adverse reactions for 60 minutes. At 30 minutes post administration, vital signs (e.g., systolic and diastolic blood pressure, heart rate, and temperature) are measured. If no systemic adverse reactions or unusual local site reactions are observed, the subjects will proceed to Stage 2 administration.

After Stage 2 administration, subjects will be monitored over the course of 30 minutes for unusual local site reactions and systemic adverse reactions. At 30 minutes post-administration, vital signs are assessed. If no evidence of systemic adverse reactions or unusual local site reactions occur, subjects are free to leave the clinic and are contacted 24 hours later for a brief telephone or in-person safety follow-up visit (e.g., Visit 2, Day 2). Subjects are instructed to keep the injection sites clean, uncovered, and to not scratch or rub the area. Subjects are asked to return to the clinic for an in-person assessment of the skin test result at various time points, starting with 48 hours post kin test administration (e.g., Visit 3, Day 3), followed by two subsequent visits at 72 hours (e.g., Visit 4, Day 4) and 96 hours (e.g., Visit 5, Day 5) post skin test administration. Subjects have two telephone safety follow-up visits (e.g., Visit 6, Day 30 and Visit 7, Day 180). The duration of the study from pre-screening contact to the last safety monitoring is approximately 6 months.

Assessment of anti-SARS-CoV-2 antibody titers are performed for the detection and quantification of antibodies to SARS-CoV-2 (for e.g., by enzyme-linked immunosorbent assay (ELISA)). Flow cytometric analysis of SARS-CoV-2-specific T-cell responses are measured, along with in vitro positive controls (e.g., benign (common cold) Coronavirus-specific peptides). To characterize Th17 response, IL-17 is evaluated using an enzyme-linked immune absorbent spot assay (ELISpot). A T-MAP assay is performed to aid the identification of individuals with an adaptive T-cell immune response to SARS-CoV-2, which indicates recent or prior infection with SARS-CoV-2.

Additional in vitro positive controls that are tested include Candida albican peptides and a PepMix CEFX Ultra SuperStim Pool (PM-CEFX) containing tetanus, herpes simplex virus, human papillomavirus, measles, mumps, rubella, and polio peptides, are analyzed to confirm that subjects have intact T-cell immunity and are no immunodeficient.

Primary Efficacy Endpoint of the study includes maximal area of induration (e.g., >5 mm) at injection sites on the volar forearms at 48 hours, 72 hours and 96 hours post skin test administration. Secondary Efficacy Endpoints of the study include: 1) Correlation of the presence or absence of DTH reactions with clinical history to estimate sensitivity and specificity of the peptide pools as a marker of active or recovered infection relative to clinical history of infection; 2) Correlation of DTH reaction to adaptive T-cell immune response to SARS-CoV-2 assessed by immunoSEQ® T-MAP™ COVID; 3) Characterization of Th1 and Th2 responses to DTH reaction by measuring cytokine production and cell surface T-cell phenotype markers by intracellular cytokine staining; and 4) Characterization of Th17 response to DTH reaction by ELISpot. Exploratory efficacy endpoints include: 1) Correlation of DTH reaction with in vitro assays of immune response including anti-SARS-CoV-2 levels; 2) Correlation of DTH reaction with flow cytometry of PBMCs (e.g., SARS-CoV-2-specific T-cell responses); and 3) Comparison of DTH reactions in recovered subjects from SARS-CoV-2 infection (e.g., Cohort 2) with COVID-19 vaccine recipients (e.g., Cohort 3).

What is claimed is:

1. A method for detecting SARS-CoV-2 cell-mediated immunity in a subject, the method comprising administering to the skin of the subject one or more peptides, at least one, at least five, at least ten, at least fifteen, at least twenty, at least twenty-five, at least thirty of the one or more peptides being selected from the group consisting of peptides being characterized by the amino acid sequences set forth in SEQ ID NO: 1-358 and inspecting the skin region in the area of the administration for an immune reaction, wherein the presence of an immune reaction is indicative of the subject having developed cell-mediated immunity to SARS-CoV-2.

2. The method of claim 1, wherein the administration is selected from the group consisting of cutaneous, intradermal, transdermal and subcutaneous administration.

3. The method of claim 1, wherein the one, five, ten, fifteen, twenty, twenty-five, thirty or more peptides are selected from the group consisting of peptides being characterized by any one of the amino acid sequences set forth in SEQ ID NO: 1-10, 12-14, 17, 18, 20-25, 27, 28, 30-39, 41, 43, 44 and 292, and optionally one or more sequences characterized by the amino acid sequences set forth in SEQ ID NO: 11, 16, 19, 26, 29, 40, 42, and 45.

4. The method of claim 1, wherein the one, five, ten, twenty, twenty-five, thirty or more peptides are selected from the group consisting of peptides being characterized by the amino acid sequences set forth in SEQ ID NO: 47, 48, 50-55, 59, 60, 62-64, 66-70, 72, 74, 75, 77, 79-81, 83, 84, 86, 87, 90-94, 96, 99-101, 103, 110, 115-125, 129, 131-133, 136-138, 140, 142, 144, 146, 148,149, 153-155, 158, 159, 294, 299, 300, 303, 308, 309, 311, 312, 317-321, 324, 325, 330-334, 337-340, and 354-357, and optionally one or more peptides being characterized by the amino acid sequences set forth in SEQ ID NO: 46, 47, 58, 61, 71, 76, 89, 105-109, 111, 113, 114, 126, 128, 130, 139, 141, 147, 151, 152, 160-167, and 265.

5. The method of claim 1, wherein the one or more peptides are selected from the group consisting of peptides being characterized by the amino acid sequences set forth in SEQ ID NO: 170, 175, 176, 178, 179, 181, 190, and 342-350, and optionally one or more peptides being characterized by the amino acid sequences set forth in SEQ ID NO: 168, 171 and 173.

6. The method of claim 1, wherein the one, five, ten, fifteen, twenty, twenty-five or thirty or more peptides are selected from the group consisting of peptides being characterized by the amino acid sequences set forth in SEQ ID NO: 235-248, 250, 252-255, 257-261, 268, 278-281, 290, 291, 351-353 and 358, and optionally one or more peptides comprising the amino acid sequences set forth in SEQ ID NO: 251, 266, 272, 274, 275, 277, 282 and 283.

7. The method of claim 1, wherein the one, five, ten, fifteen or more peptides are selected from the group consisting of peptides being characterized by the amino acid sequences set forth in SEQ ID NO: 293, 295-298, 301, 302, 304-306, 313-316, 322, 328, 329.

8. The method of claim 1, wherein the one or more peptides excludes a superantigen region.

9. The method of claim 1, wherein the one or more peptides excludes a peptide sequence similar to or the same as another coronavirus spike protein.

10. The method of any one of claim 1, wherein the immune reaction is a reaction in or on the skin.

11. The method of claim 10, wherein the immune reaction comprises an induration having a mean diameter of more than about 3 mm in or on the skin.

12. The method of claim 1, wherein the one or more peptides are administered to the skin with a syringe, a microneedle patch or a lancet.

13. The method of claim 12, wherein inspecting of the skin region takes place within 24-72 hours after administration of the one or more peptides.

14. The method of claim 1, wherein the amount of the each of the one or more peptides is 0.01 to 50 μg.

15. A method for detecting a SARS-CoV-2 infection in a subject, the method comprising detecting a cell-mediated immune response in the subject according to the method of claim 1, wherein if a cell-mediated immune response is observed, the subject is infected with SARS-CoV-2.

16. A method to measure the durability of a cell-mediated immune response weeks, months or years following natural infection or vaccination, the method comprising detecting a cell-mediated immune response in the subject according to the method of claim 1.

17. A combination or pool of peptides comprising at least one or more of the peptides with the amino acid sequence of SEQ ID NO: 1-14, 16-48, 50-55, 57-64, 66-72, 74-77, 79-81, 83, 84, 86, 87, 89-94, 96, 99-101, 103, 105-111, 113-126, 128-133, 136-142, 144, 146-149, 151-155, 158-168, 170, 171, 173, 175, 176, 178, 179, 181, 190, 235-248, 250-255, 257-261, 266, 268, 265, 272, 274, 275, 277-283, 290-306, 308-322, 324, 325, 328, 328, 330-334, 337-340, and 342-358.

18. The combination or pool of peptides of claim 17, wherein the peptides are selected from the group consisting of peptides being characterized by the amino acid sequences set forth in SEQ ID NO: 1-10, 12-14, 17, 18, 20-25, 27, 28, 30-39, 41, 43, 44 and 292, and optionally one or more peptides of SEQ ID NO: 11, 16, 19, 26, 29, 40, 42 and 45.

19. The combination or pool of peptides of claim 17, wherein the peptides are selected from the group consisting of peptides being characterized by the amino acid sequences set forth in SEQ ID NO: 47, 48, 50-55, 59, 60, 62-64, 66-70, 72, 74, 75, 77, 79-81, 83, 84, 86, 87, 90-94, 96, 99-101, 103, 110, 115-125, 129, 131-133, 136-138, 140, 142, 144, 146, 148,149, 153-155, 158, 159, 294, 299, 300, 303, 308, 309, 311, 312, 317-321, 324, 325, 330-334, 337-340, and 354-357, and optionally one or more peptides of SEQ ID NO: 46, 47, 58, 61, 71, 76, 89, 105-109, 111, 113, 114, 126, 128, 130, 139, 141, 147, 151, 152, 160-167, and 265.

20. The combination or pool of peptides of claim 17, wherein the peptides are selected from the group consisting of peptides being characterized by the amino acid sequences set forth in SEQ ID NO: 170, 175, 176, 178, 179, 181, 190, and 342-350, and optionally one or more peptides of SEQ ID NO: 168, 171, and 173.

21. The combination or pool of peptides of claim 17, wherein the peptides are selected from the group consisting of peptides being characterized by the amino acid sequences set forth in SEQ ID NO: 235-248, 250, 252-255, 257-261, 268, 278-281, 290, 291, 351-353 and 358, and optionally one or more peptides of SEQ ID NO: 251, 266, 272, 274, 275, 277, 282 and 283.

22. The combination or pool of peptides of claim 17, wherein the peptides are selected from the group consisting of peptides being characterized by the amino acid sequences set forth in SEQ ID NO: 293, 295-298, 301, 302, 304-306, 313-316, 322, 327 and 328.

23. The method of claim 1, wherein the combination or pool of peptides of claim 18 are administered to the skin of the subject.

24. The method of claim 1, wherein the combination or pool of peptides of claim 19 are administered to the skin of the subject.

25. The method of claim 1, wherein the combination or pool of peptides of claim 20 are administered to the skin of the subject.

26. The method of claim 1, wherein the combination or pool of peptides of claim 21 are administered to the skin of the subject.

27. The method of claim 1, wherein the combination or pool of peptides of claim 22 are administered to the skin of the subject.
